(19) United States
(12) Patent Application Publication
(10) Pub. No.: US 2014/0045832 A1
(43) Pub. Date: Feb. 13, 2014

(54) Insulin-Like Growth Factor-1 Receptor Inhibitors

(71) Applicants: Sarala Balachandran, New Delhi (IN); Christopher J. Dinsmore, Boston, MA (US); Abhijit Roychowdhury, Mumbai (IN); Rajiv Sharma, Mumbai (IN); Ram Asrey Vishwakarma, Jammu (IN)

(72) Inventors: Sarala Balachandran, New Delhi (IN); Christopher J. Dinsmore, Boston, MA (US); Abhijit Roychowdhury, Mumbai (IN); Rajiv Sharma, Mumbai (IN); Ram Asrey Vishwakarma, Jammu (IN)

(73) Assignees: PIRAMAL ENTERPRISES LIMITED, Mumbai (IN); MERCK SHARP & DOHME CORP., Rahway, NJ (US)

(21) Appl. No.: 14/113,166

(22) PCT Filed: Apr. 19, 2012
(86) PCT No.: PCT/US2012/034188

§ 371 Date: Oct. 21, 2013

Related U.S. Application Data
(60) Provisional application No. 61/477,937, filed on Apr. 21, 2011.

(51) Int. Cl. C07D 413/14; C07D 413/12

(52) U.S. Cl. CPC C07D 413/14; C07D 413/12
USPC 514/232.5; 544/130; 514/235.2; 544/144; 544/80

(57) ABSTRACT

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety. The present invention is also related to the pharmaceutically acceptable salts, hydrates and stereoisomers of these compounds.

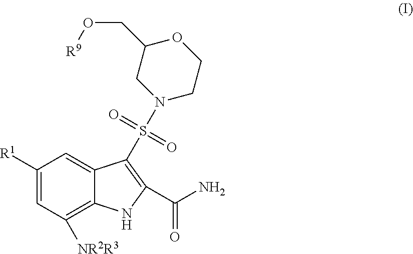

### TECHNICAL FIELD OF INVENTION

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety.

### BACKGROUND OF THE INVENTION

Protein kinases (PKs) are enzymes that catalyze the phosphorylation of hydroxy groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non life-threatening diseases such as psoriasis to extremely virulent diseases such as glioblastoma (brain cancer). PKs can be broken into two classes, the protein tyrosine kinases (PTKs) and the serine-threonine kinases (STKs).

Certain growth factor receptors exhibiting PK activity are known as receptor tyrosine kinases (RTKs). They comprise a large family of transmembrane receptors with diverse biological activity. At present, at least nineteen (19) distinct subfamilies of RTKs have been identified. One RTK subfamily contains the insulin receptor (IR), insulin-like growth factor I receptor (IGF-1R) and insulin receptor related receptor (IRR). IR and IGF-1R interact with insulin to activate a hetero-tetramer composed of two entirely extracellular glycosylated a subunits and two β subunits which cross the cell membrane and which contain the tyrosine kinase domain. The Insulin-like Growth Factor-1 Receptor (IGF-1R), and its ligands, IGF-1 and IGF-2, are abnormally expressed in numerous tumors, including, but not limited to, breast, prostate, thyroid, lung, hepatoma, colon, brain, neuroendocrine, and others.

Numerous IGF-1R small molecule inhibitors have been found to inhibit cancer growth in vitro, in vivo and in clinical trials. For example, BMS-754807 effectively inhibits the growth of a broad range of human tumor types in vitro, including mesenchymal (Ewing's, rhabdomyosarcoma, neuroblastoma, and liposarcoma), epothelial (breast, lung, pancreatic, colon, gastric), and hematopoietic (multiple myeloma and leukemia) tumor cell lines. Carboni et al., Mol Cancer Ther 2009; 8(12).

### SUMMARY OF THE INVENTION

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety. The present invention is also related to the pharmaceutically acceptable salts, hydrates and stereoisomers of these compounds.

### DETAILED DESCRIPTION OF THE INVENTION

The compounds of this invention are useful in the inhibition of IGF-1R or IR and are illustrated by a compound of Formula I:

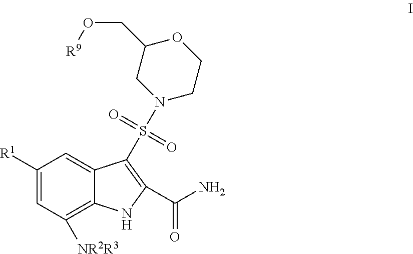

wherein:
R^{a} is independently selected from the group consisting of H and C₁-C₆ alkyl, said alkyl is optionally substituted with one to three substituents selected from R⁷;
R¹ is selected from the group consisting of:

H,

Halogen,

NO₂,

CN,

(CR^{a}₂)ₙOR⁵,

(CR^{a}₂)ₙN(R⁵)₂,

C(O)R⁵,

C(O)OR⁵,

(CR^{a}₂)ₙR⁵,

S(O)ₘR⁵,

S(O)ₘN(R⁵)₂,

SR⁵,

OS(O)ₘR⁵,

N(R⁵)C(O)R⁵,

N(R⁵)S(O)ₘR⁵, and

(CR^{a}₂)ₙC(O)N(R⁵)₂;

R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of C₁-C₆ alkyl, NR⁸C(O)R¹⁰, C(O)NR⁸R¹⁰ and C(O)OR¹²;
R⁵ is independently selected from the group consisting of

H,

C₆-C₁₀aryl,

5-10 membered heterocyclyl,

5-10 membered heterocyclenyl,

5-10 membered heteroaryl,

C₁-C₆ alkyl, and

C₃-C₈ cycloalkyl,

said aryl, heterocyclyl, heterocyclenyl, heteroaryl, alkyl and cycloalkyl is optionally substituted with one to three substituents selected from R⁷;
R⁷ is independently selected from the group consisting of:

C₁-C₆ alkyl,

Halogen,

C₁-C₆ alkoxy,

C₁-C₆ haloalkyl,

CN,

NH₂, and

NO₂;

R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl,
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl, optionally substituted with one to three substituents selected from R⁷;
R¹² is H or C₁-C₆ alkyl;
X is C₁-C₆ alkylene or C₃-C₈cycloalkylene;
Y is selected from the group consisting of H, OR¹², CN, heterocyclyl, NR⁸R¹⁰, C₃-C₈cycloalkyl, wherein C₃-C₈cycloalkyl is optionally substituted with one to three substituents selected from the group consisting of halogen, C₁-C₆ alkyl, C(O)NR⁸R¹⁰, C(O)OR¹² and NR⁸R¹¹, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;

### Z is NH, O or S;

m is 1 or 2;
n is independently 0, 1, 2, 3, 4, 5 or 6;
Or a pharmaceutically acceptable salt thereof.

In another embodiment under Formula I,

R^{a} is independently selected from the group consisting of H and C₁-C₆ alkyl, said alkyl is optionally substituted with one to three substituents selected from R⁷;
R¹ is selected from the group consisting of:

H,

Halogen,

NO₂,

CN,

(CR^{a}₂)ₙOR⁵,

(CR^{a}₂)ₙN(R⁵)₂,

C(O)R⁵,

C(O)OR⁵,

(CR^{a}₂)ₙR⁵,

S(O)ₘR⁵,

S(O)ₘN(R⁵)₂,

SR⁵,

OS(O)ₘR⁵,

N(R⁵)C(O)R⁵,

N(R⁵)S(O)ₘR⁵, and

(CR^{a}₂)ₙC(O)N(R⁵)₂;

R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of C₁-C₆ alkyl, NR⁸C(O)R¹⁰, C(O)NR⁸R¹⁰ and C(O)OR¹²;
R⁵ is independently selected from the group consisting of:

H,

C₆-C₁₀aryl,

5-10 membered heterocyclyl,

5-10 membered heterocyclenyl,

5-10 membered heteroaryl,

C₁-C₆ alkyl, and

C₃-C₈ cycloalkyl,

said aryl, heterocyclyl, heterocyclenyl, heteroaryl, alkyl and cycloalkyl is optionally substituted with one to three substituents selected from R⁷;
R⁷ is independently selected from the group consisting of:

C₁-C₆ alkyl,

Halogen,

C₁-C₆ alkoxy,

C₁-C₆ haloalkyl,

CN,

NH₂, and

NO₂;

R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl,
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl, optionally substituted with one to three substituents selected from R⁷;
R¹² is H or C₁-C₆ alkyl;
X is C₂-C₆ alkylene or C₃-C₈cycloalkylene;

Y is selected from the group consisting of H, OR¹², CN, heterocyclyl, NR⁸R¹⁰, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;

### Z is NH, O or S;

m is 1 or 2;
n is independently 0, 1, 2, 3, 4, 5 or 6.

In one embodiment,

R¹ is H, halogen, or CN;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of halogen, C₁-C₆ alkyl, NR⁸C(O)R¹⁰, C(O)NR⁸R¹⁰ and C(O)OR¹²;
R⁸ is H or C₁-C₃ alkyl;
R⁹ is selected from the group consisting of C₆-C₁₀aryl and 5-10 membered heteroaryl, said aryl or heteroaryl is optionally substituted with one to three substituents selected from R⁷;
R¹¹ is independently selected from the group consisting of C₆-C₁₀aryl and 5-10 membered heteroaryl, optionally substituted with one to three substituents selected from R⁷;
R¹² is H or C₁-C₃ alkyl;

### Z is O or S;

X is C₂-C₅ alkylene, or cyclopropylene;
And all other substituents are as defined above.

The invention also provides a compound under formula IA:

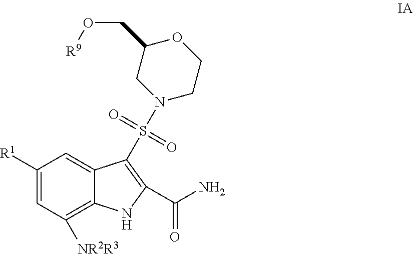

And all other substituents are as defined above.

In one embodiment, the compounds of the above formulas,

R¹ is halogen;

### R² is H;

R³ is —C(O)—X—C(O)—Y, —X—Y, —C(S)—NR¹¹R⁸, or heterocyclyl selected from the group consisting of tetrahydro-pyranyl, piperidinyl and pyrrolidinyl, and wherein the heterocyclyl is optionally substituted with halogen, C(O)NR⁸R¹⁰, C₁-C₆ alkyl, or C(O)OR¹²;

### R⁸ is H;

R⁹ is phenyl or pyridyl optionally substituted with one to three substituents selected from R⁷;
R¹¹ is phenyl optionally substituted with one to three substituents selected from R⁷;
R¹² is C₁-C₃ alkyl;
Y is selected from the group consisting of H, OR¹², CN, morpholinyl, and NH₂, wherein said morpholinyl is optionally substituted with C(O)NR⁸R¹⁰, C₁-C₆ alkyl, or C(O)OR¹²;
And all other substituents are as defined above.

The invention also provides an embodiment under Formula I and IA wherein:

R^{a} is independently selected from the group consisting of H and C₁-C₆ alkyl, said alkyl is optionally substituted with one to three substituents selected from R⁷;
R¹ is selected from the group consisting of:

H,

Halogen,

NO₂,

CN,

(CR^{a}₂)ₙOR⁵,

(CR^{a}₂)ₙN(R⁵)₂,

C(O)R⁵,

C(O)OR⁵,

(CR^{a}₂)ₙR⁵,

S(O)ₘR⁵,

S(O)ₘN(R⁵)₂,

SR⁵,

OS(O)ₘR⁵,

N(R⁵)C(O)R⁵,

N(R⁵)S(O)ₘR⁵, and

(CR^{a}₂)ₙC(O)N(R⁵)₂;

R² is H or C₁-C₆ alkyl;

R³ is

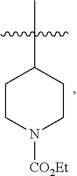 [SMILES: *C1CCN(C(=O)OCC)CC1]

### —C(Z)—X—C(O)—Y, or C(S)—NH-Ph;

R⁵ is independently selected from the group consisting of:

H,

C₆-C₁₀aryl,

5-10 membered heterocyclyl,

5-10 membered heterocyclenyl,

5-10 membered heteroaryl,

C₁-C₆ alkyl, and

C₃-C₈ cycloalkyl,

said aryl, heterocyclyl, heterocyclenyl, heteroaryl, alkyl and cycloalkyl is optionally substituted with one to three substituents selected from R⁷;
R⁷ is independently selected from the group consisting of:

C₁-C₆ alkyl,

Halogen,

C₁-C₆ alkoxy,

C₁-C₆ haloalkyl,

CN,

NH₂, and

NO₂;

R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
X is C₂-C₃ alkylene;
Y is OH or morpholinyl;

### Z is O or S;

m is 1 or 2;
n is independently 0, 1, 2, 3, 4, 5 or 6.

In one embodiment, R³ is —C(O)—CH₂CH₂—COOH or —C(O)—CH₂—CH₂—CH₂—COOH. In another embodiment, R³ is —C(S)—NH-Ph. In another embodiment, R³ is

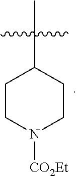 [SMILES: *C1CCN(C(=O)OCC)CC1]

In one embodiment, R² is H.

In another embodiment, R¹ is H, halogen, or CN.

In another embodiment, R⁹ is selected from the group consisting of C₆-C₁₀aryl and 5-10 membered heteroaryl, said aryl or heteroaryl is optionally substituted with one to three substituents selected from R⁷. In another embodiment, R⁹ is phenyl.

The invention also provides a compound of Formula II,

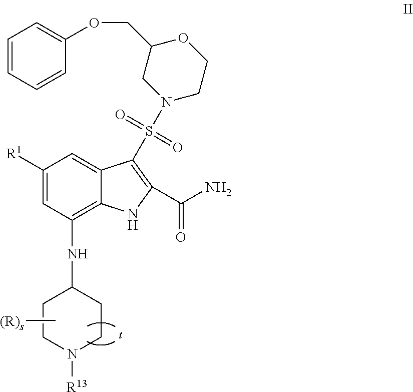

Wherein R¹ is halogen;
R¹³ is selected from the group consisting of H, C(O)NR⁸R¹⁰, C₁-C₆ alkyl, and C(O)OR¹²;
R⁸ is H or C₁-C₃ alkyl;
R¹⁰ is selected from the group consisting of C₃-C₈cycloalkyl, C₁-C₆alkyl, and C₃-C₈cycloalkylC₁-C₃alkyl,
R¹² is H or C₁-C₃ alkyl;
R is halogen;
s is 0, 1, 2, 3, or 4;
t is 0 or 1.

The invention also provides a compound of Formula IIA:

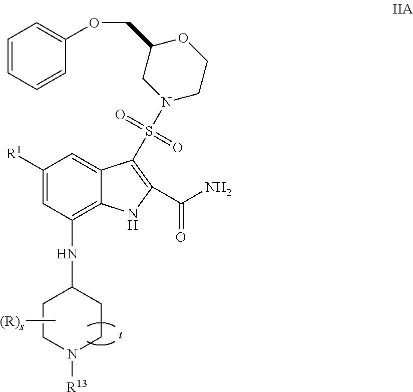

Wherein substituents are as defined above.

In one embodiment,

### R¹³ is C(O)OR¹²;

R¹² is H or C₁-C₃ alkyl.

Specific Examples of the compounds of the invention are:
(S)-4-(2-carbamoyl-5-chloro-3-(2-(phenoxymethyl)morpholinosulfonyl)-1H-indol-7-ylamino)-4-oxobutanoic acid;
(S)-5-(2-carbamoyl-5-chloro-3-(2-(phenoxymethyl)morpholinosulfonyl)-1H-indol-7-ylamino)-3,3-dimethyl-5-oxopentanoic acid;
(S)-4-(2-carbamoyl-5-chloro-3-(2-(phenoxymethyl)morpholinosulfonyl)-1H-indol-7-ylamino)-2,2-dimethyl-4-oxobutanoic acid;
(S)-5-(2-carbamoyl-5-chloro-3-(2-(phenoxymethyl)morpholinosulfonyl)-1H-indol-7-ylamino)-5-oxopentanoic acid;
2-(2-carbamoyl-5-chloro-3-((S)-2-(phenoxymethyl)morpholinosulfonyl)-1H-indol-7-ylcarbamoyl)cyclopropanecarboxylic acid;
(S)-5-chloro-7-(5-morpholino-5-oxopentanamido)-3-(2-(phenoxymethyl)morpholinosulfonyl)-1H-indole-2-carboxamide;
(S)-5-chloro-7-(2-cyanoacetamido)-3-(2-(phenoxymethyl)morpholinosulfonyl)-1H-indole-2-carboxamide;
(S)-ethyl 5-(2-carbamoyl-5-chloro-3-(2-(phenoxymethyl)morpholinosulfonyl)-1H-indol-7-ylamino)-5-oxopentanoate;
(S)-3-(2-carbamoyl-5-chloro-3-(2-(phenoxymethyl)morpholinosulfonyl)-1H-indol-7-ylamino)propanoic acid;
(S)-7-(3-amino-3-oxopropylamino)-5-chloro-3-(2-(phenoxymethyl)morpholinosulfonyl)-1H-indole-2-carboxamide;
(S)-ethyl 4-(2-carbamoyl-5-chloro-3-(2-(phenoxymethyl)morpholinosulfonyl)-1H-indol-7-ylamino)butanoate;
(S)-5-chloro-7-(2-cyanoethylamino)-3-(2-(phenoxymethyl)morpholinosulfonyl)-1H-indole-2-carboxamide;
(S)-5-chloro-3-(2-(phenoxymethyl)morpholinosulfonyl)-7-(tetrahydro-2H-pyran-4-ylamino)-1H-indole-2-carboxamide;
(S)-5-chloro-7-(cyclohexylamino)-3-(2-(phenoxymethyl)morpholinosulfonyl)-1H-indole-2-carboxamide;
(S)-5-chloro-7-(cyclohexylmethylamino)-3-(2-(phenoxymethyl)morpholinosulfonyl)-1H-indole-2-carboxamide;
(S)-methyl 4-((2-carbamoyl-5-chloro-3-(2-(phenoxymethyl)morpholinosulfonyl)-1H-indol-7-ylamino)methyl)benzoate;
(S)-5-chloro-7-(cyclopentylamino)-3-(2-(phenoxymethyl)morpholinosulfonyl)-1H-indole-2-carboxamide;
(S)-7-((1-aminocyclopentyl)methylamino)-5-chloro-3-(2-(phenoxymethyl)morpholinosulfonyl)-1H-indole-2-carboxamide;
(S)-4-((2-carbamoyl-5-chloro-3-(2-(phenoxymethyl)morpholinosulfonyl)-1H-indol-7-ylamino)methyl)benzoic acid;
(S)-7-(1-(tert-butylcarbamoyl)piperidin-4-ylamino)-5-chloro-3-(2-(phenoxymethyl)morpholinosulfonyl)-1H-indole-2-carboxamide;
(S)-5-chloro-7-(1-(cyclohexylcarbamoyl)piperidin-4-ylamino)-3-(2-(phenoxymethyl)morpholinosulfonyl)-1H-indole-2-carboxamide;
(S)-5-chloro-7-(1-(cyclohexylmethylcarbamoyl)piperidin-4-ylamino)-3-(2-(phenoxymethyl)morpholinosulfonyl)-1H-indole-2-carboxamide;
(S)-5-chloro-7-(4-fluorobenzylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-chloro-3-((S)-2-(phenoxymethyl)morpholinosulfonyl)-7-(pyrrolidin-3-ylamino)-1H-indole-2-carboxamide;
(S)-ethyl 4-(2-carbamoyl-5-fluoro-3-(2-(phenoxymethyl)morpholinosulfonyl)-1H-indol-7-ylamino)piperidine-1-carboxylate;
(S)-ethyl 4-(2-carbamoyl-5-chloro-3-(2-(phenoxymethyl)morpholinosulfonyl)-1H-indol-7-ylamino)piperidine-1-carboxylate;
(S)-5-chloro-3-(2-(phenoxymethyl)morpholinosulfonyl)-7-(3-phenylthioureido)-1H-indole-2-carboxamide; and
(S)-5-chloro-3-(2-(phenoxymethyl)morpholinosulfonyl)-7-(piperidin-4-ylamino)-1H-indole-2-carboxamide;
   Or a stereoisomer thereof;
   Or a pharmaceutically acceptable salt thereof;
   Or a pharmaceutically acceptable salt of the stereoisomer thereof.

In another embodiment, compounds of the invention are:
(S)-4-(2-carbamoyl-5-chloro-3-(2-(phenoxymethyl)morpholinosulfonyl)-1H-indol-7-ylamino)-4-oxobutanoic acid;
(S)-5-(2-carbamoyl-5-chloro-3-(2-(phenoxymethyl)morpholinosulfonyl)-1H-indol-7-ylamino)-3,3-dimethyl-5-oxopentanoic acid;
(S)-4-(2-carbamoyl-5-chloro-3-(2-(phenoxymethyl)morpholinosulfonyl)-1H-indol-7-ylamino)-2,2-dimethyl-4-oxobutanoic acid;
(S)-5-(2-carbamoyl-5-chloro-3-(2-(phenoxymethyl)morpholinosulfonyl)-1H-indol-7-ylamino)-5-oxopentanoic acid;
2-(2-carbamoyl-5-chloro-3-((S)-2-(phenoxymethyl)morpholinosulfonyl)-1H-indol-7-ylcarbamoyl)cyclopropanecarboxylic acid;
(S)-5-chloro-7-(5-morpholino-5-oxopentanamido)-3-(2-(phenoxymethyl)morpholinosulfonyl)-1H-indole-2-carboxamide;
(S)-ethyl 5-(2-carbamoyl-5-chloro-3-(2-(phenoxymethyl)morpholinosulfonyl)-1H-indol-7-ylamino)-5-oxopentanoate;
(S)-3-(2-carbamoyl-5-chloro-3-(2-(phenoxymethyl)morpholinosulfonyl)-1H-indol-7-ylamino)propanoic acid;
(S)-7-(3-amino-3-oxopropylamino)-5-chloro-3-(2-(phenoxymethyl)morpholinosulfonyl)-1H-indole-2-carboxamide;
(S)-5-chloro-7-(2-cyanoethylamino)-3-(2-(phenoxymethyl)morpholinosulfonyl)-1H-indole-2-carboxamide;
(S)-5-chloro-3-(2-(phenoxymethyl)morpholinosulfonyl)-7-(tetrahydro-2H-pyran-4-ylamino)-1H-indole-2-carboxamide;
(S)-methyl 4-((2-carbamoyl-5-chloro-3-(2-(phenoxymethyl)morpholinosulfonyl)-1H-indol-7-ylamino)methyl)benzoate;
(S)-4-(2-carbamoyl-5-chloro-3-(2-(phenoxymethyl)morpholinosulfonyl)-1H-indol-7-ylamino)methyl)benzoic acid;
(S)-7-(1-(tert-butylcarbamoyl)piperidin-4-ylamino)-5-chloro-3-(2-(phenoxymethyl)morpholinosulfonyl)-1H-indole-2-carboxamide;
(S)-5-chloro-7-(1-(cyclohexylcarbamoyl)piperidin-4-ylamino)-3-(2-(phenoxymethyl)morpholinosulfonyl)-1H-indole-2-carboxamide;
(S)-5-chloro-7-(1-(cyclohexylmethylcarbamoyl)piperidin-4-ylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-chloro-3-((S)-2-(phenoxymethyl)morpholinosulfonyl)-7-(pyrrolidin-3-ylamino)-1H-indole-2-carboxamide;
(S)-ethyl 4-(2-carbamoyl-5-fluoro-3-(2-(phenoxymethyl)morpholinosulfonyl)-1H-indol-7-ylamino)piperidine-1-carboxylate;
(S)-ethyl 4-(2-carbamoyl-5-chloro-3-(2-(phenoxymethyl)morpholinosulfonyl)-1H-indol-7-ylamino)piperidine-1-carboxylate;
(S)-5-chloro-3-(2-(phenoxymethyl)morpholinosulfonyl)-7-(3-phenylthioureido)-1H-indole-2-carboxamide; and
(S)-5-chloro-3-(2-(phenoxymethyl)morpholinosulfonyl)-7-(piperidin-4-ylamino)-1H-indole-2-carboxamide;
   Or a stereoisomer thereof;
   Or a pharmaceutically acceptable salt thereof;
   Or a pharmaceutically acceptable salt of the stereoisomer thereof.

In one embodiment, the compound is

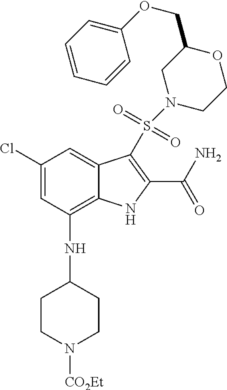 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Or a stereoisomer thereof;
Or a pharmaceutically acceptable salt thereof;
Or a pharmaceutically acceptable salt of the stereoisomer thereof.

In one embodiment, the compound is

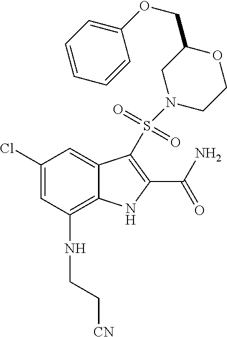 [SMILES: N#CCCNc1cc(Cl)cc2c(S(=O)(=O)N3CCO[C@H](COc4ccccc4)C3)c(C(N)=O)[nH]c12]

Or a stereoisomer thereof;
Or a pharmaceutically acceptable salt thereof;
Or a pharmaceutically acceptable salt of the stereoisomer thereof.

In another embodiment, the compound is

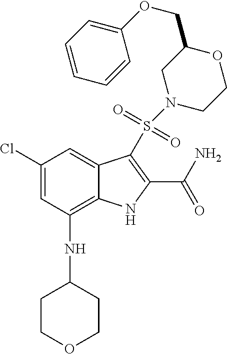 [SMILES: NC(=O)c1[nH]c2c(NC3CCOCC3)cc(Cl)cc2c1S(=O)(=O)N1CCO[C@H](COc2ccccc2)C1]

Or a stereoisomer thereof;
Or a pharmaceutically acceptable salt thereof;
Or a pharmaceutically acceptable salt of the stereoisomer thereof.

It is intended that the definition of any substituent or variable (e.g., R¹, R^{a}, n, etc.) at a particular location in a molecule be independent of its definitions elsewhere in that molecule. For example, —N(R⁴)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

### Chemical Definitions

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylaryl”, “alkylcycloalkyl” and “alkylheterocyclyl” the term “alkyl” refers to the alkyl portion of the moiety and does not describe the number of atoms in the heterocyclyl portion of the moiety. In an embodiment, if the number of carbon atoms is not specified, the “alkyl” of “alkylaryl”, “alkylcycloalkyl” and “alkylheterocyclyl” refers to C₁-C₁₂ alkyl and in a further embodiment, refers to C₁-C₆ alkyl.

The term “cycloalkyl” means a monocyclic saturated or unsaturated aliphatic hydrocarbon group having the specified number of carbon atoms. The cycloalkyl is optionally bridged (i.e., forming a bicyclic moiety), for example with a methylene, ethylene or propylene bridge. The cycloalkyl may be fused with an aryl group such as phenyl, and it is understood that the cycloalkyl substituent is attached via the cycloalkyl group. For example, “cycloalkyl” includes cyclopropyl, methyl-cyclopropyl, 2,2-dimethyl-cyclobutyl, 2-ethyl-cyclopentyl, cyclohexyl, cyclopentenyl, cyclobutenyl and so on.

In an embodiment, if the number of carbon atoms is not specified, “alkyl” refers to C₁-C₁₂ alkyl and in a further embodiment, “alkyl” refers to C₁-C₆ alkyl. In an embodiment, if the number of carbon atoms is not specified, “cycloalkyl” refers to C₃-C₁₀ cycloalkyl and in a further embodiment, “cycloalkyl” refers to C₃-C₇ cycloalkyl. In an embodiment, examples of “alkyl” include methyl, ethyl, n-propyl, i-propyl, n-butyl, t-butyl and i-butyl.

The term “alkylene” means a hydrocarbon diradical group having the specified number of carbon atoms. For example, “alkylene” includes —CH₂—, —CH₂CH₂— and the like. In an embodiment, if the number of carbon atoms is not specified, “alkylene” refers to C₁-C₁₂ alkylene and in a further embodiment, “alkylene” refers to C₁-C₆ alkylene.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

“Alkenylene” means a diradical group of an alkenyl group that is defined above. For example, “alkenylene” includes —CH₂—CH₂—CH═CH—CH₂, —CH═CH—CH₂ and the like.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

“Aryl” is intended to mean any stable monocyclic, bicyclic or tricyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In one embodiment, “aryl” is an aromatic ring of 6 to 14 carbons atoms, and includes a carbocyclic aromatic group fused with a 5- or 6-membered cycloalkyl group such as indan. Examples of carbocyclic aromatic groups include, but are not limited to, phenyl, naphthyl, e.g. 1-naphthyl and 2-naphthyl; anthracenyl, e.g. 1-anthracenyl, 2-anthracenyl; phenanthrenyl; fluorenonyl, e.g. 9-fluorenonyl, indanyl and the like.

The term heteroaryl, as used herein, represents a stable monocyclic, bicyclic or tricyclic ring of up to 7 atoms in each ring, wherein at least one ring is aromatic and contains carbon and from 1 to 4 heteroatoms selected from the group consisting of O, N and S. In another embodiment, the term heteroaryl refers to a monocyclic, bicyclic or tricyclic aromatic ring of 5- to 14-ring atoms of carbon and from one to four heteroatoms selected from O, N, or S. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. Additional examples of heteroaryl include, but are not limited to pyridyl, e.g., 2-pyridyl (also referred to as α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like.

In an embodiment, “heteroaryl” may also include a “fused polycyclic aromatic”, which is a heteroaryl fused with one or more other heteroaryl or nonaromatic heterocyclic ring. Examples include, quinolinyl and isoquinolinyl, e.g. 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g. 2-benzofuranyl and 3-benzofuranyl; dibenzofuranyl, e.g. 2,3-dihydrobenzofuranyl; dibenzothiophenyl; benzothienyl, e.g. 2-benzothienyl and 3-benzothienyl; indolyl, e.g. 2-indolyl and 3-indolyl; benzothiazolyl, e.g., 2-benzothiazolyl; benzooxazolyl, e.g., 2-benzooxazolyl; benzimidazolyl, e.g. 2-benzoimidazolyl; isoindolyl, e.g. 1-isoindolyl and 3-isoindolyl; benzotriazolyl; purinyl; thianaphthenyl, pyrazinyl and the like.

“Heterocyclyl” means a non-aromatic saturated monocyclic, bicyclic, tricyclic or spirocyclic ring system comprising up to 7 atoms in each ring. Preferably, the heterocyclyl contains 3 to 14, or 5 to 10 ring atoms, in which one or more of the atoms in the ring system is an element other than carbon, for example, nitrogen, oxygen, phosphor or sulfur, alone or in combination. There are no adjacent oxygen and/or sulfur atoms present in the ring system. Preferred heterocyclyls contain about 5 to about 6 ring atoms. The heterocycle may be fused with an aromatic aryl group such as phenyl or heterocyclenyl. The prefix aza, oxa or thia before the heterocyclyl root name means that at least a nitrogen, oxygen or sulfur atom, respectively, is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone:

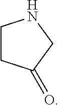

In describing the heteroatoms contained in a specified heterocyclyl group, the expression, “having one to x heteroatoms selected from the group of N, O, P and S” (wherein x is an a specified integer), for example, means that each heteroatom in the specified heterocyclyl is independently selected from the specified selection of heteroatoms. Attachment of a heterocyclyl substituent can occur via a carbon atom or via a heteroatom.

“Heterocyclenyl” means a non-aromatic monocyclic, bicyclic, tricyclic or spirocyclic ring system comprising up to 7 atoms in each ring. Preferably, the heterocyclenyl contains 3 to 14, or 5 to 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen, phosphor or sulfur atom respectively is present as a ring atom. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. An example of such a moiety is pyrrolidinone:

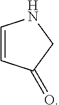

In describing the heteroatoms contained in a specified heterocyclenyl group, the expression, “having one to x heteroatoms selected from the group of N, O, P and S” (wherein x is an a specified integer), for example, means that each heteroatom in the specified heterocyclenyl is independently selected from the specified selection of heteroatoms.

It should also be noted that tautomeric forms such as, for example, the moieties:

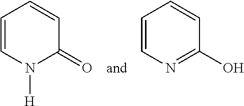 [SMILES: O=c1cccc[nH]1 ; Oc1ccccn1]

are considered equivalent in certain embodiments of this invention.

An “alkylaryl group” is an alkyl group substituted with an aryl group, for example, a phenyl group. Suitable aryl groups are described herein and suitable alkyl groups are described herein. The bond to the parent moiety is through the aryl group.

An “alkylheteroaryl group” is an alkyl group substituted with a heteroaryl group. Suitable heteroaryl groups are described herein and suitable alkyl groups are described herein. The bond to the parent moiety is through the heteroaryl group.

An “alkylheterocyclyl group” is an alkyl group substituted with a heterocyclyl group. Suitable heterocyclyl groups are described herein and suitable alkyl groups are described herein. The bond to the parent moiety is through the heterocyclyl group.

An “alkylheterocyclenyl group” is an alkyl group substituted with a heterocyclenyl group. Suitable heterocyclenyl groups are described herein and suitable alkyl groups are described herein. The bond to the parent moiety is through the heterocyclenyl group.

An “alkylcycloalkyl group” is an alkyl group substituted with a cycloalkyl group. Suitable cycloalkyl groups are described herein and suitable alkyl groups are described herein. The bond to the parent moiety is through the cycloalkyl group.

An “arylalkyl group” is an aryl group substituted with an alkyl group, for example, a phenyl group. Suitable aryl groups are described herein and suitable alkyl groups are described herein. The bond to the parent moiety is through the alkyl group.

A “heteroarylalkyl group” is a heteroaryl group substituted with an alkyl group. Suitable heteroaryl groups are described herein and suitable alkyl groups are described herein. The bond to the parent moiety is through the alkyl group.

A “heterocyclylalkyl group” is a heterocyclyl group substituted with an alkyl group. Suitable heterocyclyl groups are described herein and suitable alkyl groups are described herein. The bond to the parent moiety is through the alkyl group.

A “heterocyclenylalkyl group” is a heterocyclenyl group substituted with an alkyl group. Suitable heterocyclenyl groups are described herein and suitable alkyl groups are described herein. The bond to the parent moiety is through the alkyl group.

A “cycloalkylalkyl group” is a cycloalkyl group substituted with an alkyl group. Suitable cycloalkyl groups are described herein and suitable alkyl groups are described herein. The bond to the parent moiety is through the alkyl group.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

An “alkylamino group” as used herein, is an alkyl group that is attached to a compound via a nitrogen.

As used herein, an “arylalkylamino group” is an arylalkyl group that is attached to a compound via a nitrogen on the alkyl portion of the arylalkyl.

An “alkylsulfonyl group” as used herein, is an alkyl group that is attached to a compound via the sulfur of a sulfonyl group.

When a moiety is referred to as “unsubstituted” or not referred to as “substituted” or “optionally substituted”, it means that the moiety does not have any substituents. When a moiety is referred to as substituted, it denotes that any portion of the moiety that is known to one skilled in the art as being available for substitution can be substituted. The phrase “optionally substituted with one or more substituents” means, in one embodiment, one substituent, two substituents, three substituents, four substituents or five substituents. For example, the substitutable group can be a hydrogen atom that is replaced with a group other than hydrogen (i.e., a substituent group). Multiple substituent groups can be present. When multiple substituents are present, the substituents can be the same or different and substitution can be at any of the substitutable sites. Such means for substitution are well known in the art. For purposes of exemplification, which should not be construed as limiting the scope of this invention, some examples of groups that are substituents are: alkyl, alkenyl or alkynyl groups (which can also be substituted, with one or more substituents), alkoxy groups (which can be substituted), a halogen or halo group (F, Cl, Br, I), hydroxy, nitro, oxo, —CN, —COH, —COOH, amino, azido, N-alkylamino or N,N-dialkylamino (in which the alkyl groups can also be substituted), N-arylamino or N,N-diarylamino (in which the aryl groups can also be substituted), esters (—C(O)—OR, where R can be a group such as alkyl, aryl, etc., which can be substituted), ureas (—NHC(O)—NHR, where R can be a group such as alkyl, aryl, etc., which can be substituted), carbamates (—NHC(O)—OR, where R can be a group such as alkyl, aryl, etc., which can be substituted), sulfonamides (—NHS(O)₂R, where R can be a group such as alkyl, aryl, etc., which can be substituted), alkylsulfonyl (which can be substituted), aryl (which can be substituted), cycloalkyl (which can be substituted) alkylaryl (which can be substituted), alkylheterocyclyl (which can be substituted), alkylcycloalkyl (which can be substituted), and aryloxy.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

As used herein, the term “composition” is intended to encompass a product comprising the specified ingredients in the specified amounts, as well as any product which results, directly or indirectly, from combination of the specified ingredients in the specified amounts.

### Isotopes

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. For instance those compounds labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Additionally, isotopic substitution of a compound at a site where epimerization occurs may slow or reduce the epimerization process and thereby retain the more active or efficacious form of the compound for a longer period of time.

### Stereochemistry

When bonds to the chiral carbon are depicted as straight lines in the Formulas of the invention, it is understood that both the (R) and (S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the compounds of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

### Pharmaceutically Acceptable Salts

For use in medicine, the salts of the compounds of Formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. In one embodiment, the acids are citric, hydrobromic, hydrochloric, maleic, phosphoric, sulfuric or tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

Abbreviations, which may be used in the description of the chemistry and in the Examples that follow, include
Ac₂O Acetic anhydride;
AcOH Acetic acid;
AIBN 2,2′-Azobisisobutyronitrile;
Ar Aryl;
BINAP 2,2′-Bis(diphenylphosphino)-1,1′ binaphthyl;
Bn Benzyl;
BOC/Boc tert-Butoxycarbonyl;
BSA Bovine Serum Albumin;
CAN Ceric Ammonia Nitrate;
CBz Carbobenzyloxy;
CI Chemical Ionization;
DBAD Di-tert-butyl azodicarboxylate;
DBU 1,8-Diazabicyclo[5.4.0]undec-7-ene;
DCC 1,3-Dichlorohexylcarbodiimide;
DCE 1,2-Dichloroethane;
DCM Dichloromethane;
DIEA N,N-Diisopropylethylamine;
DMAP 4-Dimethylaminopyridine;
DME 1,2-Dimethoxyethane;
DMF N,N-Dimethylformamide;
DMSO Methyl sulfoxide;
DPPA Diphenylphosphoryl azide;
DTT Dithiothreitol;
EDC 1-(3-Dimethylaminopropyl)-3-ethyl-carbodiimide-hydrochloride;
EDTA Ethylenediaminetetraacetic acid;
ELSD Evaporative Light Scattering Detector;
ES Electrospray;
ESI Electrospray ionization;
Et₂O Diethyl ether;
Et₃N Triethylamine;
EtOAc Ethyl acetate;
EtOH Ethanol;
FAB Fast Atom Bombardment;
HEPES 4-(2-Hydroxyethyl)-1-piperazineethanesulfonic acid;
HMPA Hexamethylphosphoramide;
HOAc Acetic acid;
HOBt 1-Hydroxybenzotriazole hydrate;
HOOBt 3-Hydroxy-1,2,2-benzotriazin-4(3H)-one;
HPLC High-performance liquid chromatography;
HRMS High Resolution Mass Spectroscopy;
KOtBu Potassium tert-butoxide;
LAH Lithium aluminum hydride;
LCMS Liquid Chromatography Mass Spectroscopy;
MCPBA m-Chloroperoxybenzoic acid;
Me Methyl;
MeOH Methanol;
MP-Carbonate Macroporous polystyrene carbonate;
Ms Methanesulfonyl;
MS Mass Spectroscopy;
MsCl Methanesulfonyl chloride;
n-Bu n-butyl;
n-Bu₃P Tri-n-butylphosphine;
NaHMDS Sodium bis(trimethylsilyl)amide;
NBS N-Bromosuccinimide;
NMM N-methylmorpholine;
NMR Nuclear Magnetic Resonance;
Pd(PPh₃)₄ Palladium tetrakis(triphenylphosphine);
Pd₂(dba)₃ Tris(dibenzylideneacetone)dipalladium (0);
Ph phenyl;
PMSF α-Toluenesulfonyl fluoride;
PS-DCC Polystyrene dicyclohexylcarbodiimide;
PS-DMAP Polystyrene dimethylaminopyridine;
PS-NMM Polystyrene N-methylmorpholine;
Py or pyr Pyridine;
PYBOP Benzotriazol-1-yloxytripyrrolidinophosphonium
(or PyBOP) hexafluorophosphate;
RPLC Reverse Phase Liquid Chromatography;
RT Room Temperature;
SCX SPE Strong Cation Exchange Solid Phase Extraction;
t-Bu tent-Butyl;
TBAF Tetrabutylammonium fluoride;
TBSCl tert-Butyldimethylsilyl chloride;
TFA Trifluoroacetic acid;
THF Tetrahydrofuran;
TIPS Triisopropylsilyl;
TMS Tetramethylsilane; and
Tr Trityl.

### Utility

In another aspect, this present invention relates to a method of modulating the catalytic activity of PKs (protein kinases) in a mammal in need thereof comprising contacting the PK with a compound of Formula I.

As used herein, the term “modulation” or “modulating” refers to the alteration of the catalytic activity of receptor tyrosine kinases (RTKs), cellular tyrosine kinases (CTKs) and serine-threonine kinases (STKs). In particular, modulating refers to the activation of the catalytic activity of RTKs, CTKs and STKs, preferably the activation or inhibition of the catalytic activity of RTKs, CTKs and STKs, depending on the concentration of the compound or salt to which the RTKs, CTKs or STKs is exposed or, more preferably, the inhibition of the catalytic activity of RTKs, CTKs and STKs.

The term “catalytic activity” as used herein refers to the rate of phosphorylation of tyrosine under the influence, direct or indirect, of RTKs and/or CTKs or the phosphorylation of serine and threonine under the influence, direct or indirect, of STKs.

The term “contacting” as used herein refers to bringing a compound of this invention and a target PK together in such a manner that the compound can affect the catalytic activity of the PK, either directly; i.e., by interacting with the kinase itself, or indirectly; i.e., by interacting with another molecule on which the catalytic activity of the kinase is dependent. Such “contacting” can be accomplished “in vitro,” i.e., in a test tube, a petri dish or the like. In a test tube, contacting may involve only a compound and a PK of interest or it may involve whole cells. Cells may also be maintained or grown in cell culture dishes and contacted with a compound in that environment. In this context, the ability of a particular compound to affect a PK related disorder; i.e., the IC₅₀ of the compound, defined below, can be determined before use of the compounds in vivo with more complex living organisms is attempted. For cells outside the organism, multiple methods exist, and are well known to those skilled in the art, to get the PKs in contact with the compounds including, but not limited to, direct cell microinjection and numerous transmembrane carrier techniques.

The above-referenced PK is selected from the group comprising an RTK, a CTK or an STK in another aspect of this invention. Preferably, the PK is an RTK.

Furthermore, it is an aspect of this invention that the receptor tyrosine kinase (RTK) whose catalytic activity is modulated by a compound of this invention is selected from the group comprising EGF, HER2, HERS, HER4, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-1R, FGFR-3R and FGFR-4R. Preferably, the RTK is preferably, the receptor protein kinase is selected from IR, IGF-1R, or IRR.

In addition, it is an aspect of this invention that the cellular tyrosine kinase whose catalytic activity is modulated by a compound of this invention is selected from the group consisting of Src, Frk, Btk, Csk, Abl, ZAP70, Fes, Fps, Fak, Jak, Ack, Yes, Fyn, Lyn, Lck, Blk, Hck, Fgr and Yrk.

Another aspect of this invention is that the serine-threonine protein kinase whose catalytic activity is modulated by a compound of this invention is selected from the group consisting of CDK2 and Raf.

In another aspect, this invention relates to a method for treating or preventing a PK-related disorder in a mammal in need of such treatment comprising administering to the mammal a therapeutically effective amount of one or more of the compounds described above. In a further aspect, this invention relates to a method for treating or preventing cancer in a patient comprising administering to the mammal a therapeutically effective amount of one or more of the compounds described above. The invention also provides compounds of the invention or pharmaceutical compositions of the compounds for the treatment of cancer, and use of the compounds of the invention for the preparation of a medicament for the treatment of cancer.

As used herein, “PK-related disorder,” “PK driven disorder,” and “abnormal PK activity” all refer to a condition characterized by inappropriate (i.e., diminished or, more commonly, excessive) PK catalytic activity, where the particular PK can be an RTK, a CTK or an STK. Inappropriate catalytic activity can arise as the result of either: (1) PK expression in cells which normally do not express PKs; (2) increased PK expression leading to unwanted cell proliferation, differentiation and/or growth; or, (3) decreased PK expression leading to unwanted reductions in cell proliferation, differentiation and/or growth. Excessive-activity of a PK refers to either amplification of the gene encoding a particular PK or its ligand, or production of a level of PK activity which can correlate with a cell proliferation, differentiation and/or growth disorder (that is, as the level of the PK increases, the severity of one or more symptoms of a cellular disorder increase as the level of the PK activity decreases).

“Treat,” “treating” or “treatment” with regard to a PK-related disorder refers to alleviating or abrogating the cause and/or the effects of a PK-related disorder.

As used herein, the terms “prevent”, “preventing” and “prevention” refer to a method for barring a mammal from acquiring a PK-related disorder in the first place.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The protein kinase-related disorder may be selected from the group comprising an RTK, a CTK or an STK-related disorder in a further aspect of this invention. Preferably, the protein kinase-related disorder is an RTK-related disorder.

In yet another aspect of this invention, the above referenced PK-related disorder may be selected from the group consisting of an EGFR-related disorder, a PDGFR-related disorder, an IGFR-related disorder and a flk-related disorder.

The above referenced PK-related disorder may be a cancer selected from, but not limited to, astrocytoma, basal or squamous cell carcinoma, brain cancer, neuroblastoma, gliobastoma, liposarcoma, bladder cancer, breast cancer, colorectal cancer, colon cancer, gastric cancer, chrondrosarcoma, cervical cancer, adrenal cancer, choriocarcinoma, esophageal cancer, endometrial carcinoma, erythroleukemia, leukemia, multiple myeloma, Ewing's sarcoma, gastrointestinal cancer, head and neck cancer, hepatoma, glioma, hepatocellular carcinoma, leukemia, leiomyoma, melanoma, non-small cell lung cancer, neural cancer, ovarian cancer, pancreatic cancer, prostate cancer, renal cell carcinoma, rhabdomyosarcoma, small cell lung cancer, thyoma, thyroid cancer, testicular cancer and osteosarcoma in a further aspect of this invention. More preferably, the PK-related disorder is a cancer selected from brain cancer, breast cancer, prostate cancer, colorectal cancer, small cell lung cancer, non-small cell lung cancer, renal cell carcinoma or endometrial carcinoma.

Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

Included within the scope of the present invention is a pharmaceutical composition, which is comprised of a compound of Formula I as described above and a pharmaceutically acceptable carrier. The present invention also encompasses a method of treating or preventing cancer in a mammal in need of such treatment which is comprised of administering to said mammal a therapeutically effective amount of a compound of Formula I. Types of cancers which may be treated using compounds of Formula I include, but are not limited to, astrocytoma, basal or squamous cell carcinoma, brain cancer, gliobastoma, bladder cancer, breast cancer, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyona, melanoma, non-small cell lung cancer, neural cancer, ovarian cancer, pancreatic cancer, prostate cancer, renal cell carcinoma, rhabdomyosarcoma, small cell lung cancer, thymona, thyroid cancer, testicular cancer and osteosarcoma in a further aspect of this invention. More preferably, the cancer being treated is selected from breast cancer, prostate cancer, colorectal cancer, small cell lung cancer, non-small cell lung cancer, renal cell carcinoma, or endometrial carcinoma.

The above-referenced PK-related disorder may be an IGFR-related disorder selected from diabetes, an autoimmune disorder, Alzheimer's and other cognitive disorders, a hyperproliferation disorder, aging, cancer, acromegaly, Crohn's disease, endometriosis, diabetic retinopathy, restenosis, fibrosis, psoriasis, osteoarthritis, rheumatoid arthritis, an inflammatory disorder and angiogenesis in yet another aspect of this invention.

A method of treating or preventing retinal vascularization which is comprised of administering to a mammal in need of such treatment a therapeutically effective amount of compound of Formula I is also encompassed by the present invention. Methods of treating or preventing ocular diseases, such as diabetic retinopathy and age-related macular degeneration, are also part of the invention.

Also included within the scope of the present invention is a method of treating or preventing inflammatory diseases, such as rheumatoid arthritis, psoriasis, contact dermatitis and delayed hypersensitivity reactions, as well as treatment or prevention of bone associated pathologies selected from osteosarcoma, osteoarthritis, and rickets.

Other disorders which might be treated with compounds of this invention include, without limitation, immunological and cardiovascular disorders such as atherosclerosis.

The invention also contemplates the use of the instantly claimed compounds in combination with a second compound selected from the group consisting of:

1) an estrogen receptor modulator,

2) an androgen receptor modulator,

3) retinoid receptor modulator,

4) a cytotoxic agent,

5) an antiproliferative agent,

6) a prenyl-protein transferase inhibitor,

7) an HMG-CoA reductase inhibitor,

8) an HIV protease inhibitor,

9) a reverse transcriptase inhibitor, and

10) angiogenesis inhibitor.

A preferred angiogenesis inhibitor is selected from the group consisting of a tyrosine kinase inhibitor, an inhibitor of epidermal-derived growth factor, an inhibitor of fibroblast-derived growth factor, an inhibitor of platelet derived growth factor, an MMP inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGF. Preferred estrogen receptor modulators are tamoxifen and raloxifene.

Also included in the scope of the claims is a method of treating cancer, which comprises administering a therapeutically effective amount of a compound of Formula I in combination with a compound selected from the group consisting of:

1) an estrogen receptor modulator,

2) an androgen receptor modulator,

3) retinoid receptor modulator,

4) a cytotoxic agent,

5) an antiproliferative agent,

6) a prenyl-protein transferase inhibitor,

7) an HMG-CoA reductase inhibitor,

8) an HIV protease inhibitor,

9) a reverse transcriptase inhibitor, and

10) angiogenesis inhibitor.

And yet another embodiment is the method of treating cancer using the combination discussed above, in combination with radiation therapy.

And yet another embodiment of the invention is a method of treating cancer which comprises administering a therapeutically effective amount of a compound of Formula I in combination with paclitaxel or trastuzumab. The PKs whose catalytic activity is modulated by the compounds of this invention include protein tyrosine kinases of which there are two types, receptor tyrosine kinases (RTKs) and cellular tyrosine kinases (CTKs), and serine-threonine kinases (STKs). RTK-mediated signal transduction, is initiated by extracellular interaction with a specific growth factor (ligand), followed by receptor dimerization (or conformational changes in the case of IR, IGF-1R or IRR), transient stimulation of the intrinsic protein tyrosine kinase activity, autophosphorylation and subsequent phosphorylation of other substrate proteins. Binding sites are thereby created for intracellular signal transduction molecules and lead to the formation of complexes with a spectrum of cytoplasmic signaling molecules that facilitate the appropriate cellular response (e.g., cell division, metabolic effects on the extracellular microenvironment, etc.). See Schlessinger and Ullrich, 1992, Neuron 9:303-391.

It has been shown that tyrosine phosphorylation sites, on growth factor receptors, function as high-affinity binding sites for SH2 (src homology) domains of signaling molecules. Fantl et al., 1992, Cell 69:413-423; Songyang et al., 1994, Mol., Cell. Biol. 14:2777-2785); Songyang et al., 1993, Cell 72:767-778; and Koch et al., 1991, Science 252:668-678. Another signaling molecule domain, which interacts with phosphorylated tyrosines, is termed a PTB domain. Blaikie et al., 1994, J. Biol. Chem. 269:32031-32034; Gustafson et al., 1995, Mol. Cell. Biol., 15:2500-25008; Kavanaugh and Williams, 1994, Science 266:1862-1865. Several intracellular substrate proteins that associate with RTKs have been identified. They may be divided into two principal groups: (1) substrates which have a catalytic domain; and (2) substrates which lack such domain, but which serve as adapters and associate with catalytically active molecules. Songyang et al., 1993, Cell 72:767-778. The specificity of the interactions between receptors and SH2 domains of their substrates is determined by the amino acid residues immediately surrounding the phosphorylated tyrosine residue. Differences in the binding affinities between SH2 or PTB domains and the amino acid sequences surrounding the phosphotyrosine residues on particular receptors are consistent with the observed differences in their substrate phosphorylation profiles. Songyang et al., 1993, Cell 72:767-778. These observations suggest that the function of each RTK is determined not only by its pattern of expression and ligand availability, but also by the array of downstream signal transduction pathways that are activated by a particular receptor. Thus, phosphorylation provides an important regulatory step, which determines the selectivity of signaling pathways recruited by specific growth factor receptors, as well as differentiation factor receptors.

STKs, being primarily cytosolic, affect the internal biochemistry of the cell, often as a down-stream response to a PTK event. STKs have been implicated in the signaling process which initiates DNA synthesis and subsequent mitosis leading to cell proliferation.

Thus, PK signal transduction results in, among other responses, cell proliferation, differentiation, growth, metabolism, and cellular mobility. Abnormal cell proliferation may result in a wide array of disorders and diseases, including the development of neoplasia such as carcinoma, sarcoma, glioblastoma and hemangioma, disorders such as leukemia, psoriasis, arteriosclerosis, arthritis and diabetic retinopathy and other disorders related to uncontrolled angiogenesis and/or vasculogenesis.

A precise understanding of the mechanism by which the compounds of this invention inhibit PKs is not required in order to practice the present invention. However, while not hereby being bound to any particular mechanism or theory, it is believed that the compounds interact with the amino acids in the catalytic region of PKs. PKs typically possess a bi-lobate structure wherein ATP appears to bind in the cleft between the two lobes in a region where the amino acids are conserved among PKs. Inhibitors of PKs are believed to bind by non-covalent interactions such as hydrogen bonding, van der Waals forces and ionic interactions in the same general region where the aforesaid ATP binds to the PKs. The compounds disclosed herein may have utility as in vitro assays for such proteins as well as exhibiting in vivo therapeutic effects through interaction with such proteins.

In another aspect, the protein kinase (PK), the catalytic activity of which is modulated by contact with a compound of this invention, is a protein tyrosine kinase (PTK), more particularly, a receptor protein tyrosine kinase (RTK). Among the RTKs whose catalytic activity can be modulated with a compound of this invention, or salt thereof, are, without limitation, EGF, HER2, HER3, HER4, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-2R, FGFR-3R and FGFR-4R. Most preferably, the RTK is selected from IGF-1R.

The protein tyrosine kinase whose catalytic activity is modulated by contact with a compound of this invention, or a salt or a prodrug thereof, can also be a non-receptor or cellular protein tyrosine kinase (CTK). Thus, the catalytic activity of CTKs such as, without limitation, Src, Frk, Btk, Csk, Abl, ZAP70, Fes, Fps, Fak, Jak, Ack, Yes, Fyn, Lyn, Lck, Blk, Hck, Fgr and Yrk, may be modulated by contact with a compound or salt of this invention.

Still another group of PKs which may have their catalytic activity modulated by contact with a compound of this invention are the serine-threonine protein kinases such as, without limitation, CDK2 and Raf.

This invention is also directed to compounds that modulate PK signal transduction by affecting the enzymatic activity of RTKs, CTKs and/or STKs, thereby interfering with the signals transduced by such proteins. More particularly, the present invention is directed to compounds which modulate RTK, CTK and/or STK mediated signal transduction pathways as a therapeutic approach to cure many kinds of solid tumors, including, but not limited to, carcinomas, sarcomas including Kaposi's sarcoma, erythroblastoma, glioblastoma, meningioma, astrocytoma, melonoma and myoblastoma. Treatment or prevention of non-solid tumor cancers such as leukemia are also contemplated by this invention. Indications may include, but are not limited to brain cancers, bladder cancers, ovarian cancers, gastric cancers, pancreatic cancers, colon cancers, blood cancers, breast cancers, prostrate cancers, renal cell carcinomas, lung cancer and bone cancers.

Further examples, without limitation, of the types of disorders related to inappropriate PK activity that the compounds described herein may be useful in preventing, treating and studying, are cell proliferative disorders, fibrotic disorders and metabolic disorders.

As previously mentioned, the Insulin-like Growth Factor-1 Receptor (IGF-1R) belongs to the family of transmembrane tyrosine kinase receptors such as platelet-derived growth factor receptor, the epidermal growth factor receptor, and the insulin receptor. There are two known ligands for the IGF-1R receptor. They are IGF-1 and IGF-2. As used herein, the term “IGF” refers to both IGF-1 and IGF-2. The insulin-like growth factor family of ligands, receptors and binding proteins is reviewed in Krywicki and Yee, Breast Cancer Research and Treatment, 22:7-19, 1992.

IGF/IGF-1R driven disorders are characterized by inappropriate or over-activity of IGF/IGF-1R. Inappropriate IGF activity refers to either: (1) IGF or IGF-1R expression in cells which normally do not express IGF or IGF-1R;

(2) increased IGF or IGF-1R expression leading to unwanted cell proliferation such as cancer;
(3) increased IGF or IGF-1R activity leading to unwanted cell proliferation, such as cancer; and/or over-activity of IGF or IGF-1R. Over-activity of IGF or IGF-1R refers to either an amplification of the gene encoding IGF-1, IGF-2, IGF-1R or the production of a level of IGF activity which can be correlated with a cell proliferative disorder (i.e., as the level of IGF increases the severity of one or more of the symptoms of the cell proliferative disorder increases) the bioavailability of IGF-1 and IGF-2 can also be affected by the presence or absence of a set of IGF binding presence or absence of a set of IGF binding proteins (IGF BPs) of which there are six known. Over activity of IGF/IGF-1R can also result from a down regulation of IGF-2 which contains an IGF-2 binding domain, but no intracellular kinase domain. Examples of IGF/IGF-1R driven disorders include the various IGF/IGF-1R related human malignancies reviewed in Cullen, et al., Cancer Investigation, 9(4):443-454, 1991, incorporated herein by reference in its entirety, including any drawings. IGF/IGF-1Rs clinical importance and role in regulating osteoblast function is reviewed in Schmid, Journal of Internal Medicine, 234:535-542, 1993.

Thus, IGF-1R activities include: (1) phosphorylation of IGF-1R protein; (2) phosphorylation of an IGF-1R protein substrate; (3) interaction with an IGF adapter protein; (4) IGF-1R protein surface expression. Additional IGF-1R protein activities can be identified using standard techniques. IGF-1R activity can be assayed by measuring one or more of the following activities: (1) phosphorylation of IGF-1R; (2) phosphorylation of an IGF-1R substrate; (3) activation of an IGF-1R adapter molecule; and (4) activation of downstream signaling molecules, and/or (5) increased cell division. These activities can be measured using techniques described below and known in the arts.

IGF-1R has been implicated as an absolute requirement for the establishment and maintenance of the transformed phenotype both in vitro and in vivo in several cell types (R. Baserga, Cancer Research 55:249-252, 1995). Herbimycin A has been said to inhibit the IGF-1R protein tyrosine kinase and cellular proliferation in human breast cancer cells (Sepp-Lorenzino, et al., 1994, J. Cell Biochem. Suppl. 18b: 246). Experiments studying the role of IGF-1R in transformation have used antisense strategies, dominant negative mutants, and antibodies to the IGF-1R and have led to the suggestion that IGR-1R may be a preferred target for therapeutic interventions.

IGF-1R, in addition to being implicated in nutritional support and in type-II diabetes, has also been associated with several types of cancers. For example, IGF-1 has been implicated as an autocrine growth stimulator for several tumor types, e.g. human breast cancer carcinoma cells (Arteago et al., J. Clin. Invest., 1989, 84:1418-1423) and small lung tumor cells (Macauley et al., Cancer Res., 1989, 50:2511-2517). In addition, IGF-1, while integrally involved in the normal growth and differentiation of the nervous system, also appears to be an autocrine stimulator of human gliomas. Sandberg-Nordqvist et al., Cancer Res., 1993, 53:2475-2478.

An example of IGF-2's potential involvement in colorectal cancer may be found in the up-regulation of IGF-2 mRNA in colon tumors relative to normal colon tissue. (Zhang et al., Science (1997) 276:1268-1272.) IGF-2 may also play a role in hypoxia induced neovascularization of tumors. (Minet et al., Int. J. Mol. Med. (2000) 5:253-259.) IGF-2 may also play a role in tumorigenesis through activation of an insulin receptor isoform-A. IGF-2 activation of insulin receptor isoform-A activates cell survival signaling pathways in cells but its relative contribution to tumor cell growth and survival is unknown at this time. Insulin receptor isoform-A's kinase domain is identical to the standard insulin receptor's. Scalia et al., 2001, J. Cell Biochem. 82:610-618.

The importance of IGF-IR and its ligands in cell types in culture (fibroblasts, epithelial cells, smooth muscle cells, T-lymphocytes, myeloid cells, chondrocytes and osteoblasts (the stem cells of the bone marrow)) is illustrated by the ability of IGF-1 to stimulate cell growth and proliferation. Goldring and Goldring, Eukaryotic Gene Expression, 1991, 1:301-326. In a series of recent publications, Baserga and others suggests that IGF-1R plays a central role in the mechanism of transformation and, as such, could be a preferred target for therapeutic interventions for a broad spectrum of human malignancies. Baserga, Cancer Res., 1995, 55:249-252; Baserga, Cell, 1994, 79:927-930; Coppola et al., Mol. Cell. Biol., 1994, 14:4588-4595; Baserga, Trends in Biotechnology, 1996, 14:150-152; H. M. Khandwala et al., Endocrine Reviews, 21:215-244, 2000. The predominant cancers that may be treated using a compound of the instant invention include, but are not limited to breast cancer, prostate cancer, colorectal cancer, small cell lung cancer, non-small cell lung cancer, renal cell carcinoma, or endometrial carcinoma.

IGF-1 has also been associated with retinal neovascularization. Proliferative diabetes retinopathy has been seen in some patients having high levels of IGF-1. (L. E. Smith et al., Nature Medicine, 1999, 5:1390-1395.)

Compounds of the instant invention may also be useful as anti-aging agents. It has been observed that there is a link between IGF signalling and aging. Experiments have shown that calorie-restricted mammals have low levels of insulin and IGF-1 and have a longer life span. Similar observations have been made for insects as well. (See C. Kenyon, Cell, 2001, 105:165-168; E. Strauss, Science, 2001, 292:41-43; K. D. Kimura et al., Science 1997, 277:942-946; M. Tatar et al., Science, 2001, 292:107-110).

STKs have been implicated in many types of cancer including, notably, breast cancer (Cance et al., Int. J. Cancer, 1993, 54:571-77).

The association between abnormal PK activity and disease is not restricted to cancer. For example, RTKs have been associated with diseases such as psoriasis, diabetes mellitus, endometriosis, angiogenesis, atheromatous plaque development, Alzheimer's disease, epidermal hyperproliferation, neurodegenerative diseases, age-related macular degeneration and hemangiomas. For example, EGFR has been indicated in corneal and dermal wound healing. Defects in Insulin-R and IGF-1R are indicated in type-II diabetes mellitus. A more complete correlation between specific RTKs and their therapeutic indications is set forth in Plowman et al., DN&P, 1994, 7:334-339.

As noted previously, not only RTKs but CTKs including, but not limited to, src, abl, fps, yes, fyn, lyn, lck, Zap70, blk, hck, fgr and yrk (reviewed by Bolen et al., FASEB J., 1993, 6:3403-3409) are involved in the proliferative and metabolic signal transduction pathway and thus could be expected, and have been shown, to be involved in many PTK-mediated disorders to which the present invention is directed. For example, mutated src (v-src) has been shown to be an oncoprotein (pp60v-src) in chicken. Moreover, its cellular homolog, the protooncogene pp60^{c-src} transmits oncogenic signals of many receptors. Over-expression of EGFR or HER2/neu in tumors leads to the constitutive activation of pp60^{c-src}, which is characteristic of malignant cells, but absent in normal cells. On the other hand, mice deficient in the expression of c-src exhibit an osteopetrotic phenotype, indicating a key participation of c-src in osteoclast function and a possible involvement in related disorders.

Similarly, Zap70 has been implicated in T-cell signaling which may relate to autoimmune disorders.

STKs have been associated with inflammation, autoimmune disease, immunoresponses, and hyperproliferation disorders such as restenosis, fibrosis, psoriasis, osteoarthritis and rheumatoid arthritis.

PKs have also been implicated in embryo implantation. Thus, the compounds of this invention may provide an effective method of preventing such embryo implantation and thereby be useful as birth control agents.

Finally, both RTKs and CTKs are currently suspected as being involved in hyperimmune disorders.

These and other aspects of the invention will be apparent from the teachings contained herein.

A method for identifying a chemical compound that modulates the catalytic activity of one or more of the above discussed protein kinases is another aspect of this invention. The method involved contacting cells expressing the desired protein kinase with a compound of this invention (or its salt or prodrug) and monitoring the cells for any effect that the compound has on them. The effect may be any observable, either to the naked eye or through the use of instrumentation, change or absence of change in a cell phenotype. The change or absence of change in the cell phenotype monitored may be, for example, without limitation, a change or absence of change in the catalytic activity of the protein kinase in the cells or a change or absence of change in the interaction of the protein kinase with a natural binding partner.

### Composition

Pharmaceutical compositions of the above compounds are a further aspect of this invention.

As used herein, the term “composition” is intended to encompass a product comprising the specified ingredients in the specified amounts, as well as any product which results, directly or indirectly, from combination of the specified ingredients in the specified amounts.

The present invention also encompasses a pharmaceutical composition useful in the treatment of cancer, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

### Combination Therapy

The compounds of the present invention can be administered alone or in combination with other therapies suitable for the disease or disorder being treated. Where separate dosage formulations are used, the compound and the other therapeutic agent can be administered at essentially the same time (concurrently) or at separately staggered times (sequentially). The pharmaceutical combination is understood to include all these regimens. Administration in these various ways are suitable for the present invention as long as the beneficial therapeutic effect of the compound and the other therapeutic agent are realized by the patient at substantially the same time. In an embodiment, such beneficial effect is achieved when the target blood level concentrations of each active drug are maintained at substantially the same time.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Therefore, the present invention encompasses pharmaceutical compositions comprising a therapeutically effective amount of the compound of the invention and a pharmaceutically acceptable carrier and optionally other therapeutic ingredients, such as an anti-cancer agent. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents, agents that interfere with cell cycle checkpoints, agents that interfere with receptor tyrosine kinases (RTKs) and cancer vaccines. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, diethylstibestral, tamoxifen, raloxifene, idoxifene, LY353381, LY117081, toremifene, fluoxymestero, lfulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

Other hormonal agents include: aromatase inhibitors (e.g., aminoglutethimide, anastrozole and tetrazole), luteinizing hormone release hormone (LHRH) analogues, ketoconazole, goserelin acetate, leuprolide, megestrol acetate and mifepristone.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, chlorambucil, cyclophosphamide, ifosfamide, mechlorethamine, melphalan, uracil mustard, thiotepa, busulfan, carmustine, lomustine, streptozocin, tasonermin, lonidamine, carboplatin, altretamine, dacarbazine, procarbazine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, doxorubicin, daunorubicin, idarubicin, anthracenedione, bleomycin, mitomycin C, dactinomycin, plicatomycin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include vincristine, vinblastine, vindesine, vinzolidine, vinorelbine, vindesine sulfate, 3′,4′-didehydro-4′-deoxy-8′-norvincaleukoblastine, podophyllotoxins (e.g., etoposide (VP-16) and teniposide (VM-26)), paclitaxel, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS 188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678, WO 03/39460 and WO2003/079973, WO2003/099211, WO2004/039774, WO2003/105855, WO2003/106417. In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Examples of “histone deacetylase inhibitors” include, but are not limited to, SAHA, TSA, oxamflatin, PXD101, MG98, valproic acid and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, floxuridine, methotrexate, leucovarin, hydroxyurea, thioguanine (6-TG), mercaptopurine (6-MP), cytarabine, pentostatin, fludarabine phosphate, cladribine (2-CDA), asparaginase, gemcitabine, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, erythropoietin (epoietin-α), granulocyte-CSF (filgrastin), granulocyte, macrophage-CSF (sargramostim), pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs shown as described by Bume-Jensen and Hunter, Nature, 411:355-365, 2001.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzurnab), inhibitors of IGFR, inhibitors of CD20 (rituximab), inhibitors of cytokine receptors, inhibitors of MET, inhibitors of PI3K family kinase (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573). Such agents include small molecule inhibitor compounds and antibody antagonists.

Examples of mTOR inhibitors include ridaforolimus, temsirolimus, everolimus, a rapamycin-analog. Ridaforolimus, also known as AP 23573, MK-8669 and deforolimus, is a unique, non-prodrug analog of rapmycin that has antiproliferative activity in a broad range of human tumor cell lines in vitro and in murine tumor xenograft models utilizing human tumor cell lines. Ridaforolimus has been administered to patients with advanced cancer and is currently in clinical development for various advanced malignancies, including studies in patients with advanced soft tissue or bone sarcomas. Thus far, these trials have demonstrated that ridaforolimus is generally well-tolerated with a predictable and manageable adverse even profile, and possess anti-tumor activity in a broad range of cancers. A description and preparation of ridaforolimus is described in U.S. Pat. No. 7,091,213 to Ariad Gene Therapeutics, Inc.

Temsirolimus, also known as Torisel®, is currently marketed for the treatment of renal cell carcinoma. A description and preparation of temsirolimus is described in U.S. Pat. No. 5,362,718 to American Home Products Corporation. Everolimus, also known as Certican® or RAD001, marketed by Novartis, has greater stability and enhanced solubility in organic solvents, as well as more favorable pharmokinetics with fewer side effects than rapamycin (sirolimus). Everolimus has been used in conjunction with microemulsion cyclosporin (Neoral®, Novartis) to increase the efficacy of the immunosuppressive regime.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598.

Inhibitors of COX-2 that are particularly useful in the instant method of treatment are: 3-phenyl-4-(4-(methylsulfonyl)phenyl)-2-(511)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, imatinib (STI571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-ppidylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), Duc-4, NF-1, NF-2, RB, WT1, BRCA1, BRCA2, a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, bacillus Calmette-Guerin, octreotide, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to anastrozole, letrozole and exemestane.

A compound of the instant invention may also be useful for treating or preventing cancer in combination with siRNA therapeutics.

A compound of the instant invention may also be useful for treating or preventing cancer in combination with compounds which induce terminal differentiation of the neoplastic cells. Suitable differentiation agents include the compounds disclosed in any one or more of the following references.

a) Polar compounds (Marks et al (1987); Friend, C., Scher, W., Holland, J. W., and Sato, T. (1971) Proc. Natl. Acad. Sci. (USA) 68: 378-382; Tanaka, M., Levy, J., Terada, M., Breslow, R., Rifkind, R. A., and Marks, P. A. (1975) Proc. Natl. Acad. Sci. (USA) 72: 1003-1006; Reuben, R. C., Wife, R. L., Breslow, R., Rifkind, R. A., and Marks, P. A. (1976) Proc. Natl. Acad. Sci. (USA) 73: 862-866);

b) Derivatives of vitamin D and retinoic acid (Abe, E., Miyaura, C., Sakagami, H., Takeda, M., Konno, K., Yamazaki, T., Yoshika, S., and Suda, T. (1981) Proc. Natl. Acad. Sci. (USA) 78: 4990-4994; Schwartz, E. L., Snoddy, J. R., Kreutter, D., Rasmussen, H., and Sartorelli, A. C. (1983) Proc. Am. Assoc. Cancer Res. 24: 18; Tanenaga, K., Hozumi, M., and Sakagami, Y. (1980) Cancer Res. 40: 914-919);

c) Steroid hormones (Lotem, J. and Sachs, L. (1975) Int. J. Cancer 15: 731-740);

d) Growth factors (Sachs, L. (1978) Nature (Lond.) 274: 535, Metcalf, D. (1985) Science, 229: 16-22);

e) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354);

f) Tumor promoters (Huberman, E. and Callaham, M. F. (1979) Proc. Natl. Acad. Sci. (USA) 76: 1293-1297; Lottem, J. and Sachs, L. (1979) Proc. Natl. Acad. Sci. (USA) 76: 5158-5162); and

g) inhibitors of DNA or RNA synthesis (Schwartz, E. L. and Sartorelli, A. C. (1982) Cancer Res. 42-2651-2655, Terada, M., Epner, E., Nudel, U., Salmon, J., Fibach, E., Rifkind, R. A., and Marks, P. A. (1978) Proc. Natl. Acad. Sci. (USA) 75: 2795-2799; Morin, M. J. and Sartorelli, A. C. (1984) Cancer Res 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238).

A compound of the instant invention may also be useful for treating or preventing cancer in combination with γ-secretase inhibitors.

Also included in the scope of the claims is a method of treating cancer that comprises administering a therapeutically effective amount of a compound of Formula I in combination with radiation therapy and/or in combination with a second compound selected from: an estrogen receptor modulator, an androgen receptor modulator, a retinoid receptor modulator, a cytotoxiccytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The compounds of the instant invention are useful in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxand®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); dalteparin sodium injection (Fragmin®), Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); etoposide phosphate (Etopophos®); etoposide, VP-16 (Vepesid®); everolimus tablets (Afmitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); iobenguane I 123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); Rasburicase (Elitek®); raloxifene hydrochloride (Evista²⁰⁰), Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); vorinostat (Zolinza®); and zoledronate (Zometa®).

Non-limiting examples of other suitable anti-cancer agents for combination with the instant compounds are selected from the group consisting of a Cytostatic agent, Cisplatin, Deforolimus (described in PCT publication No. 2003/064383), Doxorubicin, liposomal doxorubicin (e.g., Caelyx®, Myocet®, Doxil®), Taxotere, Taxol, Etoposide, 41) Irinotecan, Camptostar, Topotecan, Paclitaxel, Docetaxel, Epothilones, Tamoxifen, 5-Fluorouracil, Methoxtrexate, Temozolomide, cyclophosphamide, SCH 66336, R115777®, L778,123®, BMS 214662®, Iressa®, Tarceva®, Antibodies to EGFR, antibodies to IGFR (including, for example, those published in US 2005/0136063 published Jun. 23, 2005), ESK inhibitors, KSP inhibitors (such as, for example, those published in WO 2006/098962 and WO 2006/098961; ispinesib, SB-743921 from Cytokinetics), Centrosome associated protein E (“CENP-E”) inhibitors (e.g., GSK-923295), Gleevec®, Intron, Ara-C, Adriamycin, Cytoxan, Gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6 Mercaptopurine, 6 Thioguanine, Fludarabine phosphate, Oxaliplatin, Leucovirin, ELOXATINTM, Vinblastine, Vincristine, Vindesine, Bleomycin, Dactinomycin, Daunorubicin, Doxorubicin, Epirubicin, Idarubicin, Mithramycin, Deoxycoformycin, Mitomycin C, L Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, bortezomib (“Velcade”), Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225®, Satriplatin, mylotarg, Avastin, Rituxan, Panitubimab, Sutent, Sorafinib, Sprycel (dastinib), Nilotinib, Tykerb (Lapatinib) and Campath.

In one embodiment, the invention provides a method of treating cancer, the method comprising administering an amount of a Compound of the invention or a pharmaceutically acceptable salt thereof, and an amount of one additional anticancer agent selected from the group consisting of Adriamycin, Altretamine, Amidox, Aminoglutethimide, Amsacrine, Anastrazole, Antibodies to EGFR, 3-AP, Aphidicolon, Ara-C, Arsenic trioxide, L Asparaginase, Bevacizumab, Bleomycin, BMS 214662, Bortezomib, Busulfan, Campath, Camptostar, Capecitabine, Carboplatin, Carmustine, Centrosome associated protein E (“CENP-E”) inhibitors, Cetuximab, Cladribine, Chlorambucil, Chlormethine, Chlorotrianisene, Cisplatin, Clofarabine, cyclophosphamide, Cytarabine, a Cytostatic agent, Cytoxan, Dacarbazine, Dactinomycin, Daunorubicin, Dasatinib, Deforolimus, Deoxycoformycin, Didox, Diethylstilbestrol, Docetaxel, Doxorubicin, Dromostanolone, Droloxafine, Epirubicin, Epothilones, ERK inhibitors, Erlotinib, Etoposide, 17α-Ethinylestradiol, Estramustine, Exemestane, Floxuridine, Fludarabine, Fludarabine phosphate, 5-Fluorouracil, Fluoxymesterone, Flutamide, Fulvestrant, Gefitinib, Gemcitabine, Gemtuzumab ozogamcicin, Goserelin, GSK-923295, Hexamethylmelamine, Hydroxyprogesterone, Hydroxyurea, Ibritumomab Tiuxetan, Idarubicin, Ifosfamide, Imatinib mesylate, Intron, Irinotecan, ispinesib, KSP inhibitors, L778,123, Lapatinib, Leucovirin, Leuprolide, Lerozole, Letrazole, Levamisole, Liposomal Doxorubicin, Liposomal, Lomustine, Lonafarnib, Medroxyprogesteroneacetate, Megestrolacetate, Melphalan, 6 Mercaptopurine, Methoxtrexate, Methylprednisolone, Methyltestosterone, Mithramycin, Mitomycin C, Mitotane, Mitoxantrone, Navelbene, Nilotinib, Oxaliplatin, Paclitaxel, Panitubimab, Pentostatin, Pipobroman, Porfimer, Prednisolone, Prednisone propionate, Procarbazine, Reloxafine, Rituximab, Satriplatin, SB-743921, Sml1, Sorafinib, Streptozocin, Sunitinib, Tamoxifen, Taxotere, Taxol, Temozolomide, Teniposide, Testolactone, Testosterone, Tezacitabine, 6 Thioguanine, Thiotepa, Tipifarnib, Topotecan, Toremifene, Tositumomab, Trastuzumab, Triamcinolone, Triapine, Triethylenemelamine, Triethylenethiophosphoramine, Trimidox, Uracil mustard, Vinblastine, Vincristine, Vindesine, and Vinorelbine.

In one embodiment, the invention provides a method of treating cancer, the method comprising administering an amount of a Compound of the invention or a pharmaceutically acceptable salt thereof, and an amount of one or more of a MAP Kinase pathway inhibitor such as bRaf, MEK, or ERK inhibitors to a patient in need thereof.

In another embodiment, the invention provides a method of treating cancer, the method comprising administering an amount of a Compound of the invention or a pharmaceutically acceptable salt thereof, and an amount of one or more of ERK inhibitors (for example, compounds described in WO2008/156739, WO2007/070398, WO 2008/156739 and US publication 2007/0232610) to a patient in need thereof.

In one embodiment, the invention provides a method of treating cancer, the method comprising administering an amount of a Compound of the invention or a pharmaceutically acceptable salt thereof, and an amount of one or more of an anti-IGF-1R antibody. Specific anti-IGF-1R antibodies include, but are not limited to, dalotuzumab, figitumumab, cixutumumab, SHC 717454, Roche R1507, EM164 or Amgen AMG479.

The instant invention also includes a pharmaceutical composition useful for treating or preventing cancer that comprises a therapeutically effective amount of a compound of Formula I and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The use of all of these approaches in combination with the instant compounds described herein are within the scope of the present invention.

As described above, the combinations with NSAID's are directed to the use of NSAID's which are potent COX-2 inhibiting agents. For purposes of this specification an NSAID is potent if it possess an IC₅₀ for the inhibition of COX-2 of 1 μM or less as measured by the cell or microsomal assay disclosed herein.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by the cell or microsomal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999

Inhibitors of COX-2 that are particularly useful in the instant method of treatment are:
3-phenyl-4-(4-(methylsulfonyl)phenyl)-2-(5H)-furanone; and

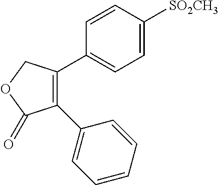 [SMILES: CS(=O)(=O)c1ccc(C2=C(c3ccccc3)C(=O)OC2)cc1]

5-chloro-3-(4-methylsulfonyl)phenyl-2-(2-methyl-5-pyridinyl)pyridine;

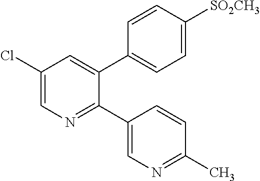 [SMILES: Cc1ccc(-c2ncc(Cl)cc2-c2ccc(S(C)(=O)=O)cc2)cn1]

or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999.

Compounds that have been described as specific inhibitors of COX-2 and are therefore useful in the present invention include, but are not limited to, the following:

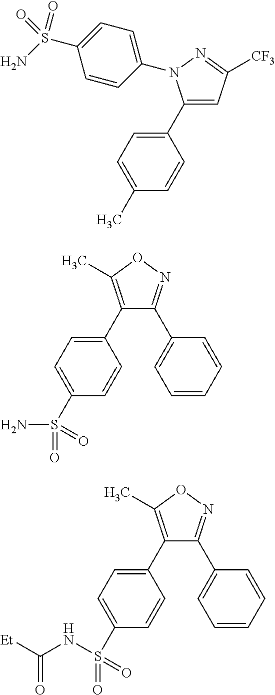

or a pharmaceutically acceptable salt thereof.

Compounds, which are described as specific inhibitors of COX-2 and are therefore useful in the present invention, and methods of synthesis thereof, can be found in the following patents, pending applications and publications: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications: U.S. Pat. No. 5,474,995 issued Dec. 12, 1995, U.S. Pat. No. 5,861,419 issued Jan. 19, 1999, U.S. Pat. No. 6,001,843 issued Dec. 14, 1999, U.S. Pat. No. 6,020,343 issued Feb. 1, 2000, U.S. Pat. No. 5,409,944 issued Apr. 25, 1995, U.S. Pat. No. 5,436,265 issued Jul. 25, 1995, U.S. Pat. No. 5,536,752 issued Jul. 16, 1996, U.S. Pat. No. 5,550,142 issued Aug. 27, 1996, U.S. Pat. No. 5,604,260 issued Feb. 18, 1997, U.S. Pat. No. 5,698,584 issued Dec. 16, 1997, and U.S. Pat. No. 5,710,140 issued Jan. 20, 1998.

### Formulations

The compounds of this invention may be administered to mammals, preferably humans, either alone or, preferably, in combination with pharmaceutically acceptable carriers, excipients or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and/or topical routes of administration.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

For oral use of a compound according to this invention, particularly for chemotherapy, the selected compound may be administered, for example, in the form of tablets or capsules, or as an aqueous solution or suspension. In the case of tablets for oral use, carriers which are commonly used include lactose and cornstarch, and lubricating agents, such as magnesium stearate, are commonly added. For oral administration in capsule form, useful diluents include lactose and dried cornstarch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The compounds of this invention may be prepared by employing reactions as shown in the following schemes, in addition to other standard manipulations that are known in the literature or exemplified in the experimental procedures. These schemes, therefore, are not limited by the compounds listed nor by any particular substituents employed for illustrative purposes. Substituent numbering, as shown in the schemes, does not necessarily correlate to that used in the claims.

### EXAMPLES

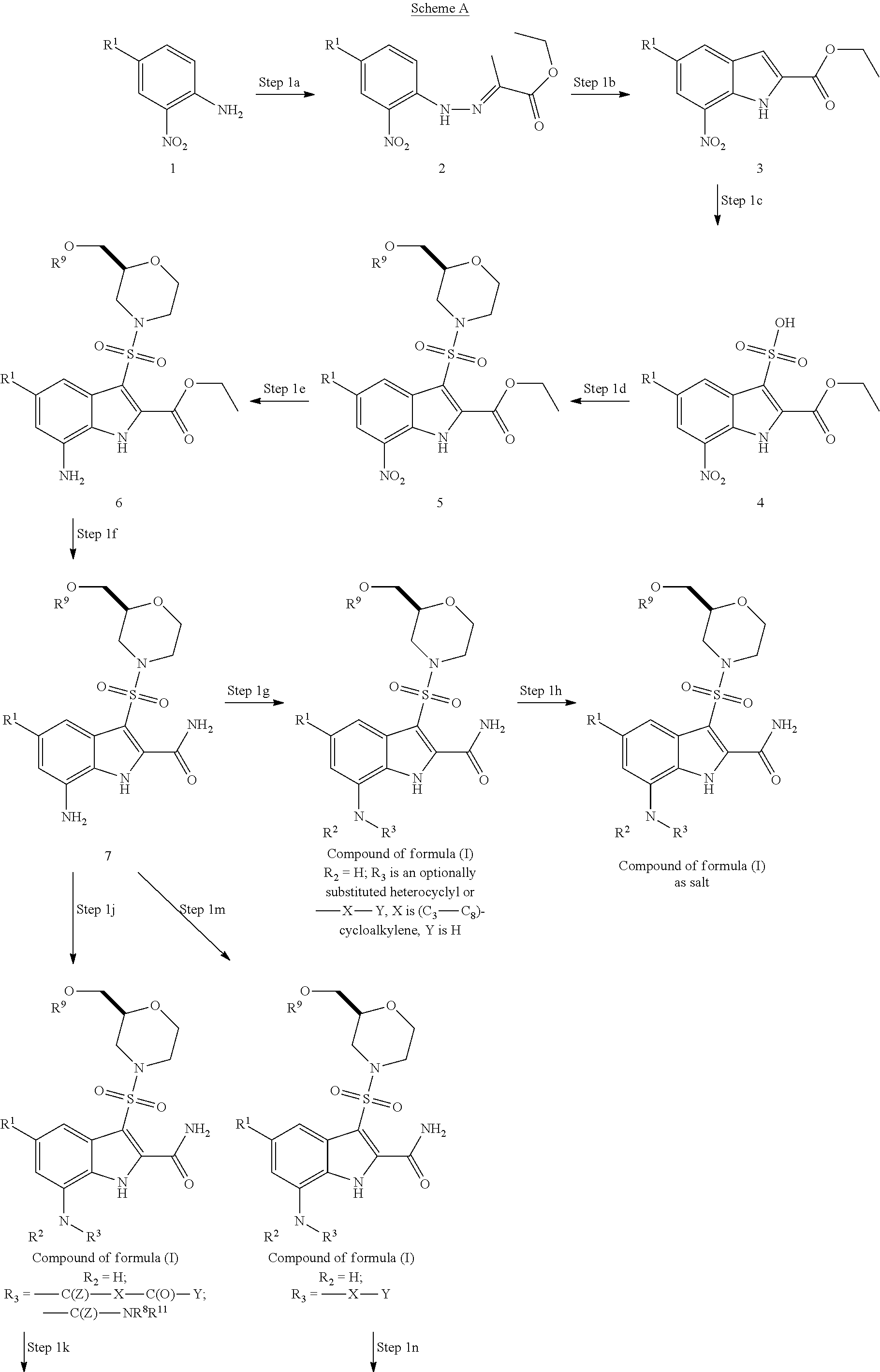

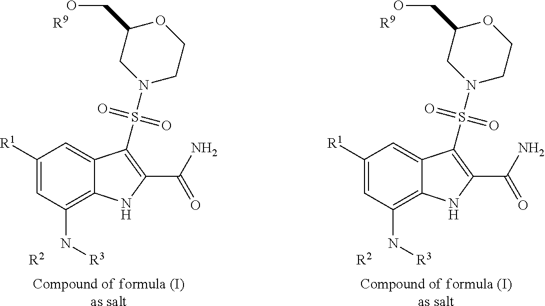

Scheme A describes the detailed process for the preparation of the compound of formula 1, the steps comprising:

### Step 1a:

Diazotisation of the compound of formula 1 (which is commercially available or may be prepared by methods, well-known in the art):

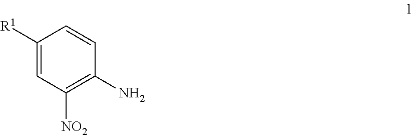

wherein R¹ is as defined in formula I, by reaction with NaNO₂ and HCl at a temperature range of −10 to 5° C., followed by a dropwise addition of the diazotized mixture to an alkaline solution of the reagent, ethyl 2-methyl-3-oxobutanoate in a base selected from KOH or NaOH in a solvent such as methanol or ethanol at a temperature range of −20° C. to −15° C. to afford the compound of formula 2;

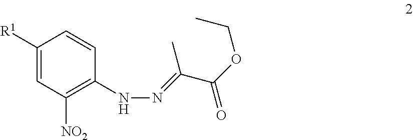

wherein R¹ is as defined in formula I.

### Step 1b:

Cyclisation of the compound of formula 2 by reaction with a Lewis acid such as ZnCl₂, AlCl₃, BF₃, P₂O₅ or polyphosphoric acid at a temperature range of 80-120° C. for 5-12 h to afford the compound of formula 3;

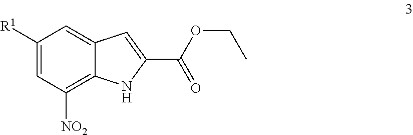

wherein R¹ is as defined in formula I.

### Step 1c:

Sulphonation of the compound of formula 3 by reaction with sulphuric acid and acetic anhydride at a temperature range of 0-30° C. for 10-20 h to afford the compound of formula 4;

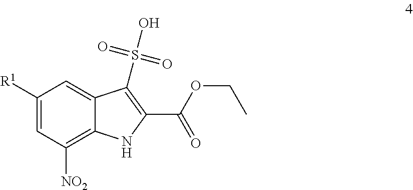

wherein R¹ is as defined in formula I.

### Step 1d:

Reaction of the compound of formula 4 with oxalyl chloride or thionyl chloride in presence of a suitable organic base selected from triethylamine or pyridine in a solvent selected from DMF, methylene dichloride or a mixture thereof at a temperature range of 25-50° C. for 1-6 h to prepare the corresponding sulphonyl chloride of the compound of formula 4, which is further reacted with the intermediate of formula E;

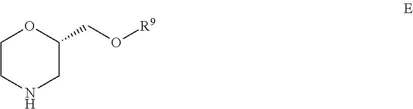

wherein R⁹ is as defined in formula I; at room temperature in presence of an organic base selected from pyridine or triethylamine in a solvent selected from dichloromethane or chloroform at room temperature (25-30° C.) for 2-12 h to afford the compound of formula 5;

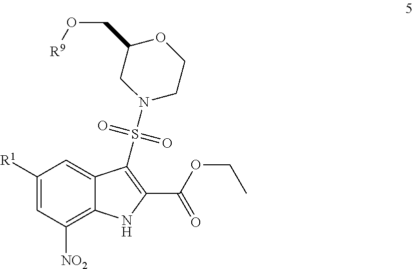

wherein R¹ and R⁹ are as defined in formula I.

### Step 1e:

Reduction of the compound of formula 5 by reaction with a reducing agent selected from Fe and NH₄Cl, Zn and HCl or SnCl₂ for 2-8 h in a suitable solvent selected from methanol, ethanol, THF, water or a mixture thereof, to afford the compound of formula 6;

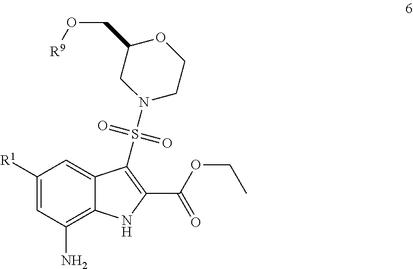

wherein R¹ and R⁹ are as defined in formula I.

### Step 1f:

Reaction of the compound of formula 6 with isopropyl alcohol and NH₃ at a temperature range of 80 to 120° C. in a sealed tube for 10-18 h or in a microwave for 10-15 min to afford the compound of formula 7;

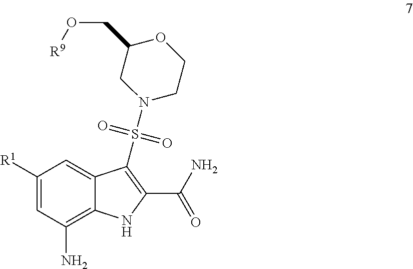

wherein R¹ and R⁹ are as defined in formula I.

### Step 1g:

Reaction of the compound of formula 7 with the reagent of formula F;

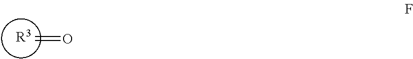

wherein R³ is an optionally substituted heterocyclyl or —X—Y wherein X is (C₃-C₈)-cycloalkylene and Y is H, as defined in Formula I; in the presence of trifluoroacetic acid in a suitable base such as sodium triacetoxy borohydride and optionally, Hunig's base; in a suitable solvent selected from dichloromethane or ethyl acetate at room temperature for 0.5-2 h to afford the compound of formula I;

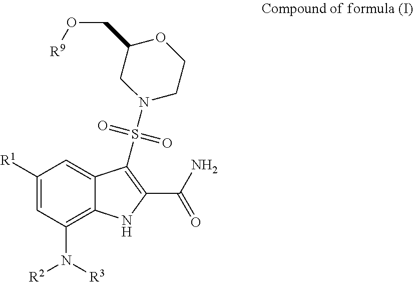

wherein R¹ and R⁹ are as defined in formula I; R² is H and R³ is an optionally substituted heterocyclyl or —X—Y wherein X is (C₃-C₈)-cycloalkylene and Y is H.

### Step 1h:

Reaction of the compound of formula I with corresponding acid selected from acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid or p-toluenesulfonic acid to afford the corresponding pharmaceutically acceptable salt of the compound of formula I.

### Step 1j:

Reaction of the compound of formula 7 with the compound of formula (R³)₂O, R³OH or R¹¹NC(Z) in a suitable solvent selected from toluene, dioxane or THF at a temperature range of 70° C. to 100° C. for about 1-4 h to afford the compound of formula I, wherein R³ is —C(Z)XC(O)Y or —C(Z)NR⁸R¹¹ where R⁸ is H and Z, X, Y and R¹¹ are as defined in formula I.

### Step 1k:

Reaction of the compound of formula I of Step 1j with corresponding acid selected from acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid or p-toluenesulfonic acid to afford the corresponding pharmaceutically acceptable salt of the compound of formula I.

### Step 1m:

Reaction of the compound of formula 7 with the compound of formula R³-halide; R³ is —X—Y wherein X and Y are as defined in formula I, in the presence of a suitable base selected from anhydrous sodium carbonate, potassium carbonate, triethylamine or pyridine to afford the compound of formula I.

### Step 1n:

Reaction of the compound of formula I of Step 1m with corresponding acid selected from acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid or p-toluenesulfonic acid to afford the corresponding pharmaceutically acceptable salt of the compound of formula I.

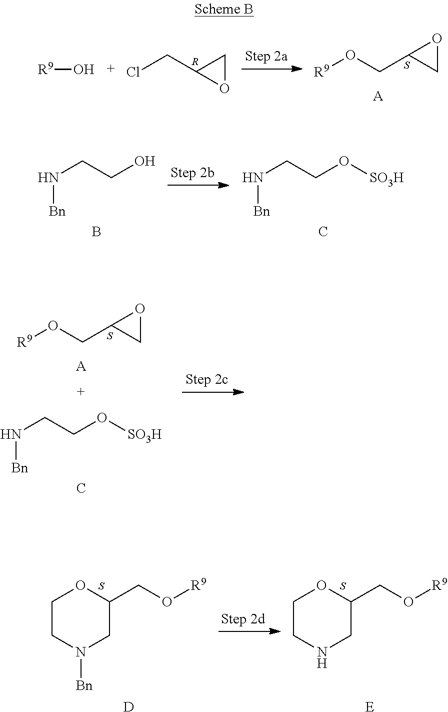

Scheme B describes the detailed process for the preparation of the compound of formula E, the steps comprising:

### Step 2a:

Reaction of the compound of formula R⁹—OH wherein R⁹ is as defined in formula 1 (which is commercially available or may be prepared by methods well known in the art) with (R)-2-(chloromethyl)oxirane in presence of a base such as aqueous NaOH or aqueous KOH and a phase transfer catalyst, tetrabutyl ammonium hydrogen sulphate at a temperature range of 80-120° C. for 1-4 h to afford the compound of formula A:

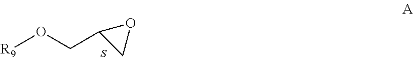

wherein R⁹ is as defined in formula I.

### Step 2b:

Reaction of the compound of formula B (commercially available) with chlorosulfonic acid in a solvent selected from chloroform, carbon tetrachloride or dichloromethane, initially at 0-10° C. during addition of the acid, followed by at room temperature for 10-16 h to afford the compound of formula C:

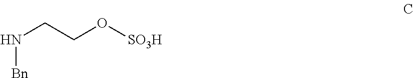 [SMILES: O=S(=O)(O)OCCNCc1ccccc1]

### Step 2c:

Reaction of the compound of formula A with the compound of formula C in presence of an aqueous base such as NaOH or KOH in a suitable solvent selected from toluene, dioxane or THF in presence of a phase transfer catalyst such as tetrabutylammoniun hydrogen sulfate at a temperature range of 30-50° C. for 10-16 h to afford the compound of formula D;

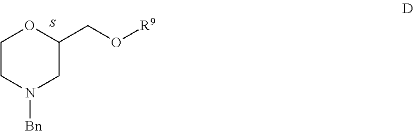

wherein R⁹ is as defined in formula I.

### Step 2d:

Debenzylation of the compound of formula D by refluxing the compound of formula D with ammonium formate and 10% Pd/C in an atmosphere of carbon dioxide in a solvent selected from ethanol or methanol at 50-70° C. for 1-3 h to afford the compound of formula E:

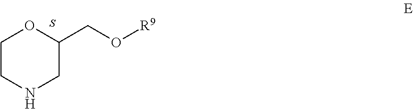

wherein R⁹ is as defined in formula I.

### Example 1

### Synthesis

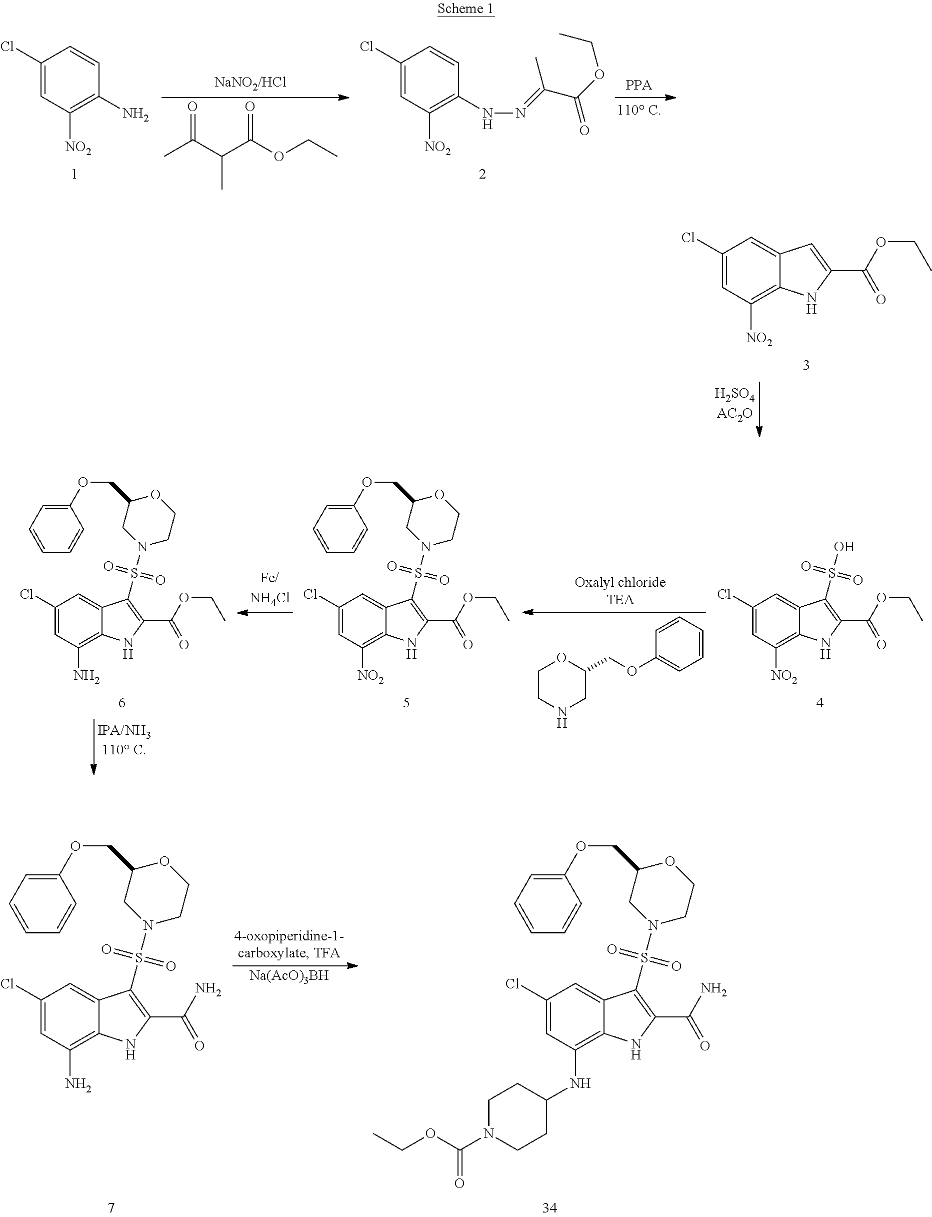

### Synthesis of ethyl 2-(2-(4-chloro-2-nitrophenyl)hydrazono)propanoate (2)

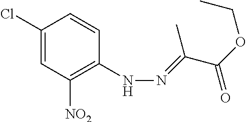 [SMILES: CCOC(=O)/C(C)=N/Nc1ccc(Cl)cc1[N+](=O)[O-]]

### Procedure:

To an ice-cold solution of 965 g of ethyl-2-methyl acetoacetate in 4.0 L of ethanol was added 1.528 kg (50%) KOH at 0 to −10° C. This mixture was then diluted with 20.0 kg of ice. Simultaneously a cold diazonium salt solution was prepared from 1.0 kg of 2-nitro-4-chloro aniline, 3.0 L of conc. HCl, 4.5 L of water and 440 g of sodium nitrite at 0 to −5° C. The diazonium salt mixture was then poured rapidly into the above ethanol solution of ethyl-2-methyl acetoacetate with constant stirring. The reaction was stirred for another 30 minutes. The solid was then filtered by suction filtration to yield crude compound 2, which on further crystallisation from ethanol gave pure compound 2.

¹H NMR (300 MHz, DMSO-d6) δ 10.87 (s, 1H), 8.19 (s, 1H), 8.01-7.99 (d, J=8.4 Hz, 1H), 7.57-7.54 (d, J=7.8 Hz, 1H), 4.37-4.35 (q, 2H), 2.24 (s, 3H), 1.40 (t, 3H). MS: [M−H]⁻: 284.0

### Synthesis of ethyl 5-chloro-7-nitro-1H-indole-2-carboxylate (3)

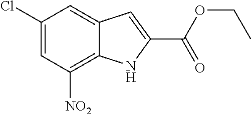 [SMILES: CCOC(=O)c1cc2cc(Cl)cc([N+](=O)[O-])c2[nH]1]

### Procedure:

Polyphosphoric acid (PPA) was heated at 110° C. and ethyl 2-(2-(4-chloro-2-nitrophenyphydrazono)propanoate (700 g) was added to the heated PPA mixture. This mixture was then stirred for 8-9 hours. The reaction mass was basified using saturated sodium carbonate and the product was extracted in ethyl acetate (1 L×5). The organic layer was washed by saturated sodium carbonate (200 mL) followed by brine (200 mL), dried over sodium sulphate and evaporated in vacuo to yield the titled compound (3).

¹H NMR (300 MHz, DMSO-d6) δ 10.31 (s, 1H), 8.27-8.26 (d, J=1.5 Hz, 1H), 8.01-8.01 (d, J=1.2 Hz, 1H), 7.30-7.27 (s, 1H), 4.51-4.44 (q, 2H), 1.48-1.41 (t, 3H). MS: [M−H]⁻: 267.0

### Synthesis of 5-chloro-2-(ethoxycarbonyl)-7-nitro-1H-indole-3-sulfonic acid (4)

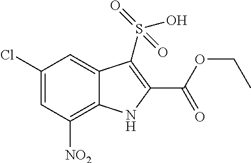 [SMILES: CCOC(=O)c1[nH]c2c([N+](=O)[O-])cc(Cl)cc2c1S(=O)(=O)O]

### Procedure:

To compound 3 (350 g) was added acetic anhydride (622 mL) at room temperature. The reaction mixture was subsequently cooled to 0-10° C., and sulphuric acid (355 mL) was added drop wise. The reaction was stirred for 12-15 hours at room temperature to ensure consumption of starting material. The solid was then filtered by suction filtration to get crude compound 3, which on crystallization by EtOAc (1-2 vol) yielded pure compound 4.

¹H NMR (300 MHz, DMSO-d6) δ 12.28 (s, 1H),s 8.357-8.351 (d, J=1.8 Hz, 1H), 8.18-8.17 (d, J=1.8 Hz, 1H), 4.33-4.25 (q, 2H), 1.33-1.29 (t, 3H). MS: [M−H]⁻: 347.0

### Synthesis of (S)-ethyl 5-chloro-7-nitro-3-(2-(phenoxymethyl)morpholinosulfonyl)-1H-indole-2-carboxylate (5)

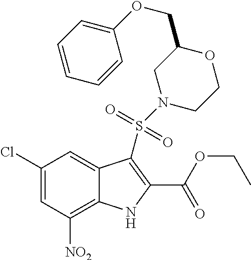 [SMILES: CCOC(=O)c1[nH]c2c([N+](=O)[O-])cc(Cl)cc2c1S(=O)(=O)N1CCO[C@H](COc2ccccc2)C1]

### Procedure:

To compound 4 (175 g) was suspended in dichloromethane (700 mL) and catalytic amount of DMF was added. The reaction mixture was cooled to 10° C. and oxalyl chloride (130 mL) was added in a drop wise fashion. The reaction mixture was stirred for 12 hours to afford the desired sulfonyl chloride. Upon completion of the reaction DCM was distilled out completely under high vacuum. Fresh DCM (500 mL), triethylamine (105 mL) and (S)-2-(phenoxymethyl)morpholine (102 g) was then added to the above solid and stirred for 4 hours to ensure coupling reaction. The DCM was evaporated in vacuo. The residue was resuspended in water (200 mL) stirred and extracted in DCM (500 mL×3). The organic layer was then washed with saturated bicarbonate (200 mL×2), brine (200 mL) and dried over sodium sulfate (20 g). The organic layer was then filtered and concentrated in vacuo to yield crude compound 5. The crude was used for the next reaction.

¹H NMR (300 MHz, DMSO-d6) δ 13.46 (s, 1H), 8.338-8.332 (d, J=1.8 Hz 1H), 8.26-8.25 (d, J=1.8 Hz, 1H), 7.29-7.24 (m, 2H), 6.95-6.88 (m, 3H), 4.41-4.34 (q, 2H), 3.98-3.93 (m, 3H), 3.81-3.77 (m, 2H), 3.67-3.58 (m, 2H), 2.60-2.49 (m, 2H), 1.32-1.28 (t, 3H). MS: [M+H]⁺: 524.0

### Synthesis of (S)-ethyl 7-amino-5-chloro-3-(2-(phenoxymethyl)morpholinosulfonyl)-1H-indole-2-carboxylate (6)

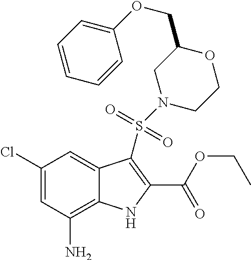 [SMILES: CCOC(=O)c1[nH]c2c(N)cc(Cl)cc2c1S(=O)(=O)N1CCO[C@H](COc2ccccc2)C1]

### Procedure:

Compound 5 (150 g), iron powder (80 g), ammonium chloride (76.5 g) was mixed in ethanol (400 mL). The reaction mixture was heated up to 80-85° C. for 6-7 hours. Ethanol was evaporated and the mixture was dissolved in chloroform (200 mL). To the chloroform layer was added water in EDTA (200 g in 200 mL). The chloroform layer was separated. The water layer was further extracted with chloroform (200 mL×2). The combined organic layer was then washed with saturated sodium bicarbonate (200 mL×2), brine (200 mL) and subsequently dried over sodium sulfate (20 g). The organic layer was then filtered and evaporated in vacuo to generate crude compound 6. This was used in the next step without any further purification.

¹H NMR (300 MHz, DMSO-d6) δ 12.66 (s, 1H), 7.29-7.24 (m, 2H), 7.17 (s, 1H), 6.95-6.88 (m, 3H), 6.52 (s, 1H), 6.00 (bs, 2H), 4.41-4.34 (q, 2H), 3.99-3.90 (m, 3H), 3.81-3.78 (m, 2H), 3.61-3.52 (m, 2H), 2.59-2.50 (m, 2H), 1.34-1.22 (t, 3H). MS: [M+H]⁺: 494.1

### Synthesis of (S)-7-amino-5-chloro-3-(2-(phenoxymethyl)morpholinosulfonyl)-1H-indole-2-carboxamide (7)

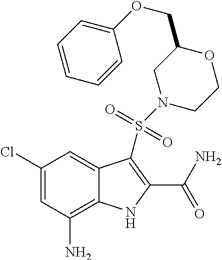 [SMILES: NC(=O)c1[nH]c2c(N)cc(Cl)cc2c1S(=O)(=O)N1CCO[C@H](COc2ccccc2)C1]

### Procedure:

Compound 6 (95 g) was dissolved in isopropyl alcohol (IPA) (900 mL) in a sealed tube and ammonia gas was passed through for 15 minutes. The tube was sealed and heated to 110° C. for 12-15 hours. The pressure was released carefully and IPA evaporated. The solid was absorbed on silica (200-400 mesh) and subjected to column chromatography. The product was eluted in 10% MeOH/chloroform to obtain compound 7 in pure form.

¹H NMR (300 MHz, DMSO-d6) δ 12.59 (s, 1H), 8.30-8.23 (d, J=21.0 Hz, 2H), 7.28-7.23 (m, 2H), 7.108-7.102 (d, J=1.8 Hz, 1H), 6.94-6.87 (m, 3H), 6.49-6.48 (d, J=1.8 Hz, 1H), 6.01 (bs, 2H), 4.03-3.94 (m, 2H), 3.90-3.79 (m, 2H), 3.68-3.46 (m, 3H), 2.50-2.31 (m, 2H). MS: [M+H]⁺: 465.1

### Compound 34: (S)-ethyl 4-((2-carbamoyl-5-chloro-3-((2-(phenoxymethyl)morpholino)sulfonyl)-1H-indol-7-yl)amino)piperidine-1-carboxylate

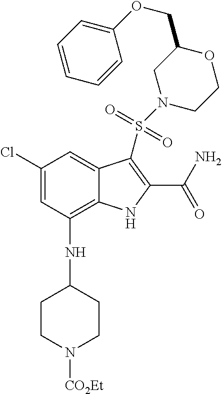 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Compound 7 (40 gm) and ethyl 4-oxopiperidine-1-carboxylate (29.51 gm) were taken in DCM (1.2 L) and the turbid solution was stirred for 20 h at RT. Subsequent to the overnight reaction TFA (33 mL) was added dropwise and stirred for 10 minutes. Following this, sodium tri-acetoxyborohydride was added and the reaction mixture stirred for another 1.5 hours. The reaction mass was concentrated and residue was dissolved in 250 mL ethyl acetate. The organic layer was washed with water (2×2.0 L) and brine (1.5 L). The organic phase was dried over sodium sulphate and concentrated in vacuo to yield crude product (56.0 g). The crude product was then subjected to column chromatography (2% MeOH/CHCl₃) to yield pure compound 34.

¹H NMR (300 MHz, DMSO-d6) δ 12.66 (s, 1H), 8.31-8.31 (d, J=12.6 Hz, 2H), 7.28-7.23 (t, J=8.1Hz, 2H), 7.14-7.13 (d, J=1.2 Hz, 1H), 6.95-6.87 (m, 2H), 6.474-6.471 (d, J=0.9 Hz, 1H), 6.38-6.36 (d, J=7.2, 1H), 4.08-3.94 (m, 2H), 3.97-3.91 (m, 4H), 3.82-3.80 (m, 2H), 3.67-3.64 (d, J=10.5 Hz, 2H), 3.58-3.43 (m, 2H), 3.07 (m, 2H), 2.45-2.30 (m, 3H), 2.02-1.98 (d, J=9.9 Hz, 2H), 1.37-1.26 (m, 2H), 1.21-1.17 (t, J=6.9 Hz, 3H). MS: [M+H]⁺: 620.2

### Methanesulfonic acid salt of (S)-ethyl 4-((2-carbamoyl-5-chloro-3-((2-(phenoxymethyl)morpholino)sulfonyl)-1H-indol-7-yl)amino)piperidine-1-carboxylate

Compound 34 (41 g) was dissolved in THF (400 mL) and methane sulfonic acid (6.35 g) was added and stirred at room temperature (RT) for 90 min. The content was concentrated to 200 mL and then 300 mL n-hexane was added and stirred till free powder was observed in the solution. The solid was filtered and washed with n-Hexane (200 mL) and dried under reduced pressure to yield pure title compound.

¹H NMR (300 MHz, DMSO-d6) δ 12.66 (s, 1H), 8.30-8.26 (d, J=13.2 Hz, 2H), 7.28-7.23 (t, J=7.5 Hz, 2H), 7.14 (s, 1H), 6.94-6.87 (m, 3H), 6.47 (s, 1H), 4.06-4.01 (m, 2H), 3.95-3.90 (m, 4H), 3.81 (m, 1H), 3.67-3.59 (m, 2H), 3.50-3.46 (m, 2H), 3.07 (m, 2H), 2.44 (s, 3H), 2.37-2.30 (m, 2H), 2.02-1.98 (d, J=10.5 Hz, 2H), 1.75 (m, 1H), 1.34-1.31 (m, 2H), 1.21-1.17 (t, J=7.2 Hz, 3H).

### Scheme for the Synthesis of (S)-2-(phenoxymethyl)morpholine

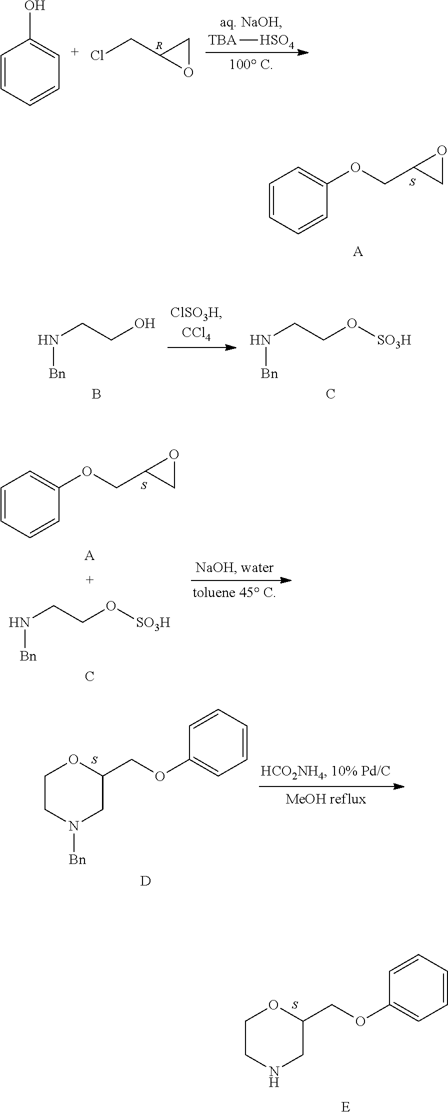

### Compound A (S)-2-phenoxymethyloxirane

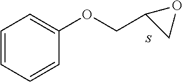

A solution of NaOH and phenol in water (143 g in 1.8 L) at ambient temperature was taken in a 2-necked RB flask fitted with a mechanical stirrer and a reflux air condenser. To this reaction mixture was then added Tetrabutylammoniumhydrogensulphate (1.5 g). R-epichlorohydrin (662 g) was added slowly over a period of 10-15 minutes along with vigorous stirring. The mixture was stirred vigorously at 90-100° C. for 1 hour. Upon completion of reaction it was extracted with 1:1 ethyl acetate: petroleum ether (1 L). The combined organic layer was concentrated in vacuo. The residue was distilled and the fraction from 115-125° C. at 2 mm (diaphragm pump) was collected (The oil bath temp was 155-160° C.) to yield the desired compound.

¹H NMR (300 MHz, CDCl₃) δ 7.28-7.34 (m, 2H), 6.93-7.03 (m, 3H), 4.255 (m, 1H), 4.00 (m, 1H), 3.390 (t, 1H), 2.95 (m, 1H), 2.785 (m, 1H). MS: [M+H] 151.

### Compound C: N-Benzyl Ethanolamine Hydrogen Sulphate

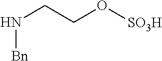 [SMILES: O=S(=O)(O)OCCNCc1ccccc1]

A solution of N-benzlyethanolamine (328 gms) in CCl₄ (2 L) was taken in a 2 necked round bottomed flask fitted with a mechanical stirrer and a dropping funnel. The reaction mixture was cooled to 0° C. Chlorosulphonic acid (256 g) was added dropwise to the solution while maintaining the reaction temp between 0-5° C. After addition was complete the mixture was then stirred at RT for 16 hours. Upon completion of the reaction, the solid was filtered washed with 1:1 EtOH:CHCl₃ (650 mL) and dried at 50° C. under high vac. (0.5 mm) for 1 hour to yield the desired product.

¹H NMR (300 MHz, D₂O) δ 7.388 (s, 5H), 4.214 (m, 4H), 3.32 (t, 2H). MS [M+H] 232

### Compound D: (S)-1-Benzyl-2-phenoxymethylmorpholine

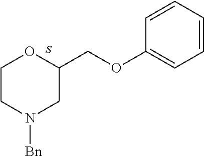

An aqueous NaOH (572 g in 1 L water) solution was charged into a 2 necked RB flask fitted with a mechanical stirrer and a dropping funnel. This was cooled to 10-15° C. To this was added N-benzyl ethanolamine hydrogen sulphate (368 g) (C) while maintaining the temperature at <20° C. The mixture was stirred at RT for 10 minutes. A solution of (S)-2-(phenoxymethyl)oxirane (A) (216 g) in toluene was added over 10-15 minutes. The mixture was stirred at 45-50° C. for 16 hours. Upon completion of reaction water (2 L) and EtOAc (2 L) was added to the reaction mixture. The organic layer was separated and washed with water and extracted with 10% aqueous HCl (2 L). The combined HCl washings were basified with NaOH to pH 9 and extracted with EtOAc (2.1 L). The EtOAc extract was washed with water (1 L), brine (1 L), dried over Na₂SO₄ and concentrated in vacuo to yield the product.

¹H NMR (300 MHz, CDCl₃) δ 7.33-7.23 (m, 7H), 6.96-6.93 (d, J=7.5 Hz, 1H), 6.90-6.88 (d, J=8.1Hz, 2H), 4.05-3.90 (m, 4H), 3.77-3.66 (t, J=11.1Hz, 1H), 3.55 (s, 2H), 3.49-2.86 (d, J=11.1Hz, 1H), 2.70-2.66 (d, J=11.1Hz, 1H), 2.274-2.187 (t, J=11.4 Hz, 1H), 2.131-2.063 (t, J=9.6 Hz, 1H), MS [M+H]: 284

### Compound E: (S)-2-(phenoxymethyl)morpholine

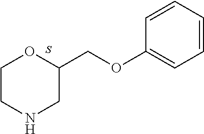

A stirred solution of compound D (210 g) in methanol (2 L) was taken in a 2 necked RB flask fitted with a mechanical stirrer and a reflux condenser. Under a bed of CO₂ (obtained by adding a small piece of dry ice to the mixture) was added 10% Pd/C. To the above reaction mixture was added ammonium formate (210 g) at ambient temp and the above reaction mixture was refluxed for 1 hour. Upon completion of reaction, the Pd—C was filtered and washed with MeOH. The filtrate was concentrated in vacuo. The residue was dissolved in EtOAc (2 L) and the organic layer was washed with water (1 L×2), dried over Na₂SO₄ and concentrated in vacuo at 60° C. for 1 hour to yield compound E.

¹H NMR (300 MHz, CDCl₃) δ 7.31-7.26 (m, 2H), 6.99-6.91 (m, 3H), 4.11-4.09 (m, 2H), d 4.047-3.990 (m, 2H), 3.977-3.656 (t, 1H), 3.091-2.740 (m, 4H). MS [M+H]: 194

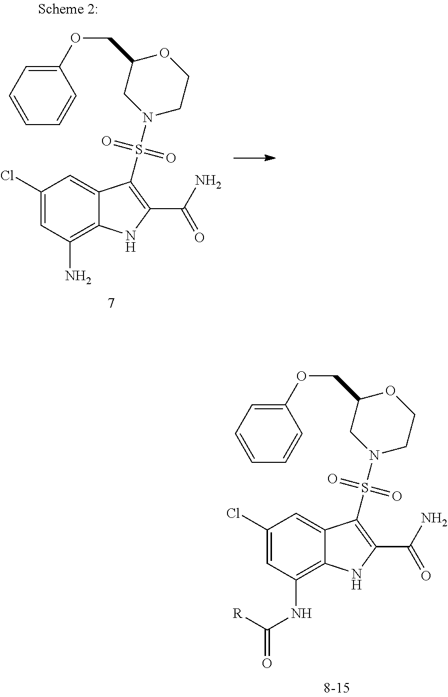

### Generic Procedure of Amide Synthesis (Scheme 2)

### Compound-8-(S)-4-((2-carbamoyl-5-chloro-3-((2-(phenoxymethyl) morpholino)sulfonyl)-1H-indol-7-yl)amino)-4-oxobutanoic acid

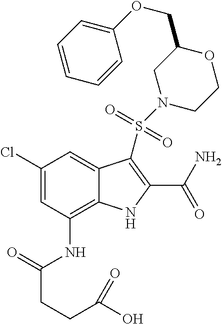 [SMILES: NC(=O)c1[nH]c2c(NC(=O)CCC(=O)O)cc(Cl)cc2c1S(=O)(=O)N1CCO[C@H](COc2ccccc2)C1]

### Procedure:

(S)-7-amino-5-chloro-3-(2-(phenoxymethyl)morpholinosulfonyl)-1H-indole-2-carboxamide (7) was dissolved (0.075 g) in toluene (5 mL) subsequent to which succinic anhydride was added and the reaction mixture was (0.020 g) heated at 110° C. for 2 hr. Upon completion of reaction toluene was evaporated in vacuo. To the residue petroleum ether (20 mL) was added and the solid filtered. The filtered solid was washed with 15 mL of petroleum ether to obtain the title compound.

¹H NMR (300 MHz, DMSO-d6) δ 12.84 (s, 1H), 12.25 (s, 1H), 10.18 (s, 1H), 8.34 (d, J=12.6 Hz, 2H), 8.13 (s, 1H), 7.65 (s, 1H), 7.28-6.87 (m, 5H), 3.94 (m, 3H), 3.81 (m, 1H), 3.70-3.49 (m, 3H), 2.71-2.60 (m, 4H), 2.44-2.27 (m, 2H).

### Compound-9-(S)-5-((2-carbamoyl-5-chloro-3-((2-(phenoxymethyl)morpholino)sulfonyl)-1H-indol-7-yl)amino)-3,3-dimethyl-5-oxopentanoic acid

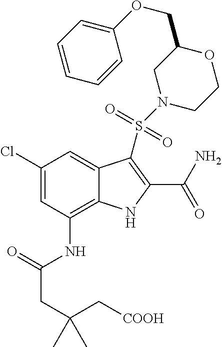 [SMILES: CC(C)(CC(=O)O)CC(=O)Nc1cc(Cl)cc2c(S(=O)(=O)N3CCO[C@H](COc4ccccc4)C3)c(C(N)=O)[nH]c12]

Following the procedure described for compound 8, replacing succinic anhydride with 4,4-dimethyldihydro-2H-pyran-2,6(3H)-dione, the title compound (9) was obtained after a simple filtration procedure.

¹H NMR (300 MHz, DMSO-d6) δ 12.85 (s, 1H), 12.04 (s, 1H), 10.01 (s, 1H), 8.37 (d, J=16.3 Hz, 2H), 8.20 (s, 1H), 7.66 (s, 1H), 7.25-6.87 (m, 3H), 3.94 (m, 3H), 3.81 (m, 2H), 3.69-3.49 (m, 4H), 2.38 (s, 4H), 1.14 (s, 6H).

### Compound-10 (S)-4-((2-carbamoyl-5-chloro-3-((2-(phenoxymethyl)morpholino)sulfonyl)-1H-indol-7-yl)amino)-2,2-dimethyl-4-oxobutanoic acid

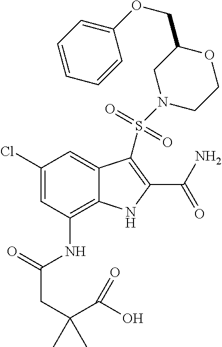 [SMILES: CC(C)(CC(=O)Nc1cc(Cl)cc2c(S(=O)(=O)N3CCO[C@H](COc4ccccc4)C3)c(C(N)=O)[nH]c12)C(=O)O]

Following the procedure described for compound 8 replacing succinic anhydride with 4,4-dimethyldihydro-2H-pyran-2,6(3H)-dione, the title compound was obtained after a simple filtration procedure.

¹H NMR (300 MHz, DMSO-d6) δ 13.06 (s, 1H), 12.09 (s, 1H), 8.31-8.25 (d, J=19.2 Hz, 2H), 7.94 (s, 1H), 7.44 (s, 1H), 7.26-6.91 (m, 5H), 3.97 (m, 2H), 3.91 (m, 1H), 3.83 (m, 1H), 3.74-3.52 (m, 4H), 2.78 (s, 2H), 2.44 (m, 2H), 1.14 (s, 6H).

### Compound-11 (S)-5-((2-carbamoyl-5-chloro-3-((2-(phenoxymethyl)morpholino)sulfonyl)-1H-indol-7-yl)amino)-5-oxopentanoic acid

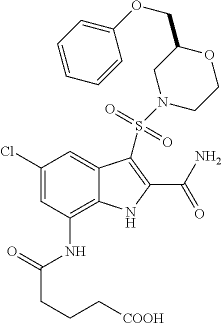 [SMILES: NC(=O)c1[nH]c2c(NC(=O)CCCC(=O)O)cc(Cl)cc2c1S(=O)(=O)N1CCO[C@H](COc2ccccc2)C1]

Following the procedure described for compound 8, replacing succinic anhydride with glutaric anhydride, the title compound was obtained after a simple filtration procedure.

¹H NMR (300 MHz, DMSO-d6) δ 12.90 (s, 1H), 12.09 (s, 1H), 10.07 (s, 1H), 8.36-8.33 (d, J=16.3 Hz, 2H), 8.15 (s, 1H), 7.66 (s, 1H), 7.28-6.87 (m, 5H), 3.95-3.90 (m, 3H), 3.81 (m, 1H), 3.70-3.49 (m, 3H), 2.40-2.32 (m, 2H), 1.89-1.65 (m, 6H).

### Compound-12: 2-((2-carbamoyl-5-chloro-3-(((S)-2-(phenoxymethyl) morpholino)sulfonyl)-1H-indol-7-yl)carbamoyl)Cyclopropanecarboxylic acid

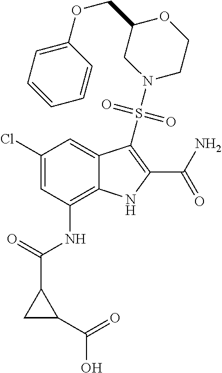 [SMILES: NC(=O)c1[nH]c2c(NC(=O)C3CC3C(=O)O)cc(Cl)cc2c1S(=O)(=O)N1CCO[C@H](COc2ccccc2)C1]

Following the procedure described for compound 8, replacing succinic anhydride with 3-oxabicyclo[3.1.0]hexane-2,4-dione, the title compound was obtained after a simple filtration procedure.

¹H NMR (300 MHz, DMSO-d6) δ 12.86 (s, 1H), 12.28 (s, 1H), 10.37 (s, 1H), 8.38-8.34 (d, J=17.6 Hz, 2H), 8.11 (s, 1H), 7.66 (s, 1H), 7.28-6.87 (m, 5H), 3.95-3.90 (m, 3H), 3.83-3.81 (m, 1H), 3.70-3.49 (m, 4H), 2.30 (m, 1H), 2.16-2.08 (m, 2H), 1.51-1.45 (m, 1H), 1.30-1.26 (m, 1H).

Compound-13 (S)-5-chloro-7-(5-morphohno-5-oxopentanamido)-3-(2-(phenoxymethyl)morpholinosulfonyl)-1H-indole-2-carboxamide

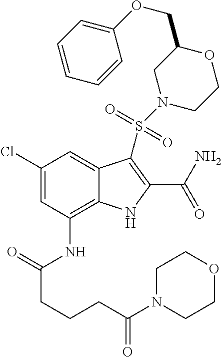 [SMILES: NC(=O)c1[nH]c2c(NC(=O)CCCC(=O)N3CCOCC3)cc(Cl)cc2c1S(=O)(=O)N1CCO[C@H](COc2ccccc2)C1]

### Procedure:

Compound 11 was dissolved in DMF (0.5 mL), to which O-(benzotriazol-1-yl)-N,N,N′,N′-tetramethyluronium tetrafluoroborate (TBTU) was added and stirred at RT for 5 minutes. To this reaction mixture morpholine (10.53 μL) was added and stirred overnight. Upon completion of reaction, ice was added to the reaction mixture and the desired product was in ethyl acetate. Ethyl acetate was evaporated to yield the title compound.

¹H NMR (300 MHz, DMSO-d6) δ 12.53 (s, 1H), 10.06 (s, 1H), 8.33 (s, 2H), 8.15 (s, 1H), 7.65 (s, 1H), 7.26 (s, 2H), 6.89 (s, 3H), 3.95 (m, 3H), 3.82-3.79 (m, 1H), 3.70-3.67 (m, 1H), 3.54 (m, 7H), 3.44 (m, 5H), 2.40 (m, 6H).

### Compound-15 (S)-ethyl 5-(2-carbamoyl-5-chloro-3-(2-(phenoxymethyl)morpholinosulfonyl)-1H-indol-7-ylamino)-5-oxopentanoate

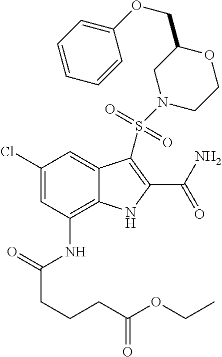 [SMILES: CCOC(=O)CCCC(=O)Nc1cc(Cl)cc2c(S(=O)(=O)N3CCO[C@H](COc4ccccc4)C3)c(C(N)=O)[nH]c12]

### Procedure:

To a solution of compound 11 in ethanol, concentrated sulfuric acid (catalytic) was added drop wise at 0° C. The reaction mixture was refluxed at 75° C. for 3 hours. Upon completion of reaction, small portion of ice was added to the reaction mixture and extracted with EtOAc. The organic layer was washed with NaHCO₃ solution and brine solution. The title compound was obtained after subjecting to column chromatography (10% MeOH/Chloroform).

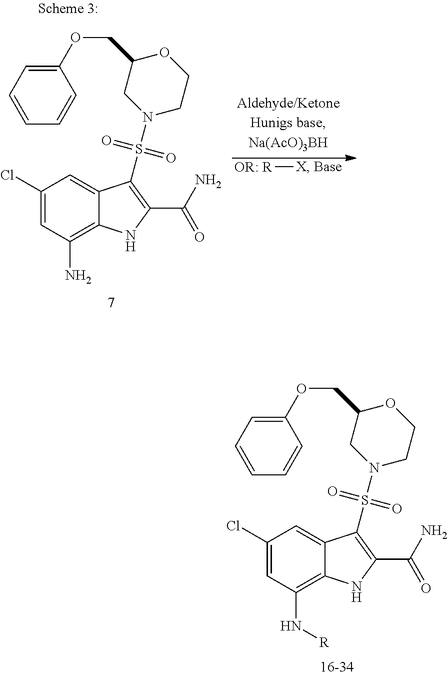

### Compound 16—(S)-3-(2-carbamoyl-5-chloro-3-(2-(phenoxymethyl)morpholinosulfonyl)-1H-indol-7-ylamino)propanoic acid

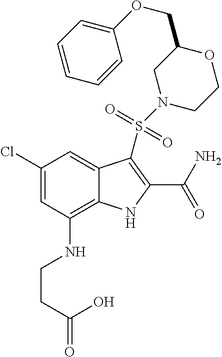 [SMILES: NC(=O)c1[nH]c2c(NCCC(=O)O)cc(Cl)cc2c1S(=O)(=O)N1CCO[C@H](COc2ccccc2)C1]

### Procedure:

The titled compound is obtained in a two step procedure. The ethyl ester intermediate ((S)-ethyl 3-(2-carbamoyl-5-chloro-3-(2-(phenoxymethyl)morpholinosulfonyl)-1H-indol-7-ylamino)propanoate) was obtained upon condensation of (S)-7-amino-5-chloro-3-(2-(phenoxymethyl)morpholinosulfonyl)-1H-indole-2-carboxamide (7) with ethyl bromopropionate in the presence of potassium carbonate under refluxing conditions. The ethyl ester intermediate ((S)-ethyl 3-(2-carbamoyl-5-chloro-3-(2-(phenoxymethyl)morpholinosulfonyl)-1H-indol-7-ylamino)propanoate) (80 mg), was dissolved in ethanol (3 mL), and subjected to hydrolysis under NaOH 1M conditions (8.5 mg) for 4 hours to obtain the desired compound. Upon completion, ethanol was evaporated. The aqueous layer was filter through celite and subsequently acidified. The acidified layer was then filtered and purified by column using 5% MeOH in chloroform to yield title compound.

¹H NMR (300 MHz, DMSO-d6) δ 12.69 (s, 1H), 12.30 (s, 1H), 8.29-8.24 (d, J=19.5 Hz, 2H), 7.28-7.23 (m, 2H), 7.16 (s, 1H), 6.94-6.87 (m, 3H), 6.53 (m, 1H), 6.36 (s, 1H), 3.98-3.90 (m, 4H), 3.81 (m, 1H), 3.67 (m, 1H), 3.41 (m, 2H), 2.72 (m, 1H), 2.63-2.58 (m, 2H), 2.18 (m, 2H).

### Compound 17: (S)-7-((3-amino-3-oxopropyl)amino)-5-chloro-3-((2-(phenoxymethyl)morpholino)sulfonyl)-1H-indole-2-carboxamide

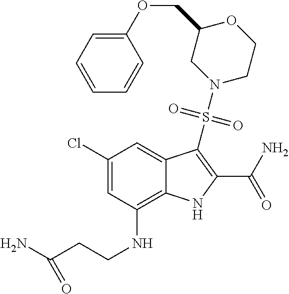 [SMILES: NC(=O)CCNc1cc(Cl)cc2c(S(=O)(=O)N3CCO[C@H](COc4ccccc4)C3)c(C(N)=O)[nH]c12]

### Procedure:

The titled compound was obtained in a two step procedure. The first step was to obtain the same ethyl ester intermediate ((S)-ethyl 3-(2-carbamoyl-5-chloro-3-(2-(phenoxymethyl)morpholinosulfonyl)-1H-indol-7-ylamino)propanoate)) as described for compound 16. This ester intermediate was subjected to saturated IPA ammonia in sealed tube at 110° C. overnight to obtain the titled compound. Upon completion of reaction IPA/ammonia was evaporated, the title compound was obtained after subjecting to column chromatography [0-5% MeOH/CHCl₃].

¹H NMR (300 MHz, DMSO-d6) δ 12.74 (s, 1H), 8.28-8.22 (d, J=13.6 Hz, 2H), 7.68 (s, 1H), 7.39-7.14 (m, 3H), 6.90-6.88 (m, 2H), 6.53 (s, 1H), 6.36 (s, 1H), 3.95-3.90 (m, 2H), 3.81 (m, 1H), 3.67-3.46 (m, 3H), 2.33 (m, 2H), 1.99-1.87 (m, 2H), 1.64-1.51 (m, 2H), 1.33-1.23 (m, 3H).

### Compound-19: (S)-5-chloro-7-((2-cyanoethyl)amino)-3-((2-(phenoxymethyl) morpholino)sulfonyl)-1H-indole-2-carboxamide

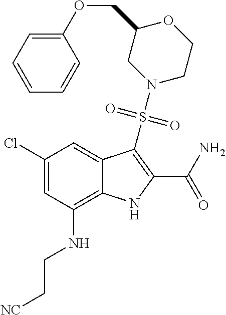 [SMILES: N#CCCNc1cc(Cl)cc2c(S(=O)(=O)N3CCO[C@H](COc4ccccc4)C3)c(C(N)=O)[nH]c12]

(S)-7-amino-5-chloro-3-(2-(phenoxymethyl)morpholinosulfonyl)-1H-indole-2-carboxamide, potassium carbonate (2.5 eq) and potassium iodide (0.005 eq) were dissolved in DMF. The reaction mixture was cooled to 0° C. and 3-bromopropanenitrile (1.5 eq) was added drop wise. The reaction mixture was stirred at 100° C. for 3 days. Upon disappearance of starting material as monitored by TLC, DMF was evaporated completely. The solid residue was dissolved in DCM and residual solid filtered off. The crude material was distilled by vacuum distillation to yield the title compound which was then subjected to column chromatography [2% MeOH in chloroform].

¹H NMR (300 MHz, DMSO-d6) δ 12.66 (s, 1H), 8.30-8.25 (d, 2H), 7.26-7.18 (m, 3H), 6.90-6.85 (m, 3H), 6.79-6.74 (m, 1H), 6.48 (s, 1H), 3.99-3.88 (m, 3H), 3.81-3.78 (m, 1H), 3.66-3.44 (m, 5H), 2.84-2.79 (m, 2H), 2.40-2.25 (m, 2H).

### Generic Procedure of Reductive Amination Reactions. (Scheme 3)

### Compound-20: (S)-5-chloro-3-((2-(phenoxymethyl)morpholino)sulfonyl)-7-((tetrahydro-2H-pyran-4-yl)amino)-1H-indole-2-carboxamide

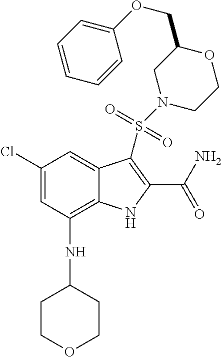 [SMILES: NC(=O)c1[nH]c2c(NC3CCOCC3)cc(Cl)cc2c1S(=O)(=O)N1CCO[C@H](COc2ccccc2)C1]

### Procedure:

(S)-7-amino-5-chloro-3-(2-(phenoxymethyl)morpholinosulfonyl)-1H-indole-2-carboxamide (7), dihydro-2H-pyran-4(3H)-one (1.5 eq) and Hunig's base (5 eq) were dissolved in DCM and the reaction mixture was stirred for 2 hr. Then sodium triacetoxy borohydride (5 eq) was added and stirring was continued for 2 days. Upon completion of reaction, the solvent was evaporated and the title compound was obtained after subjecting to column chromatography [2% MeOH in chloroform].

¹H NMR (300 MHz, DMSO-d6) δ 12.67 (s, 1H), 8.30-8.25 (d, J=29.0 Hz, 2H), 7.26-7.23 (m, 2H, 7.13 (s, 1H), 6.90-6.87 (m, 3H), 6.46 (s, 1H), 6.39-6.37 (d, J=6.3 Hz, 1H), 3.95-3.82 (m, 5H), 3.67-3.46 (m, 4H), 2.41 (m, 2H), 2.34-2.30 (m, 1H), 2.00-1.91 (m, 2H), 1.46-1.42 (m, 2H), 1.23 (m, 2H).

### Compound-23: (S)-methyl 4-(((2-carbamoyl-5-chloro-3-((2-(phenoxymethyl)morpholino)sulfonyl)-1H-indol-7-yl)amino)methyl)benzoate

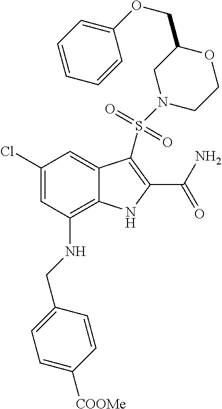 [SMILES: COC(=O)c1ccc(CNc2cc(Cl)cc3c(S(=O)(=O)N4CCO[C@H](COc5ccccc5)C4)c(C(N)=O)[nH]c23)cc1]

Following the procedure described for compound 20 and replacing dihydro-2H-pyran-4(3H)-one with ethyl 4-formylbenzoate (1.5 eq), the title compound was obtained after subjecting to column chromatography [2% MeOH in chloroform].

¹H NMR (300 MHz, DMSO-d6) δ 12.70 (s, 1H), 8.29-8.24 (d, J=15.6 Hz, 2H), 7.98-7.96 (d, J=8.1 Hz, 2H), 7.58-7.55 (d, J=8.4 Hz, 2H), 7.28-7.22 (m, 3H), 7.16 (s, 1H), 7.06 (m, 1H), 6.94-6.87 (m, 3H), 6.31 (s, 1H), 4.55-4.54 (d, J=4.8 Hz, 2H), 3.97-3.95 (m, 2H), 3.84 (m, 2H), 3.59-3.46 (m, 3H), 2.41-2.34 (m, 1H), 1.33-1.23 (m, 3H).

### Compound-26: (S)-4-(((2-carbamoyl-5-chloro-3-((2-(phenoxymethyl) morpholino)sulfonyl)-1H-indol-7-yl)amino)methyl)benzoic acid

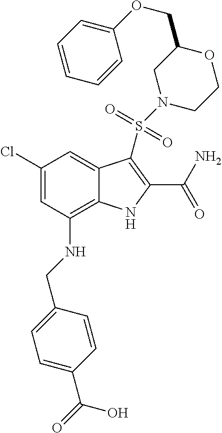 [SMILES: NC(=O)c1[nH]c2c(NCc3ccc(C(=O)O)cc3)cc(Cl)cc2c1S(=O)(=O)N1CCO[C@H](COc2ccccc2)C1]

Following the procedure described for compound 20 and replacing dihydro-2H-pyran-4(3H)-one with 4-formylbenzoic acid (1.5 eq) the title compound was obtained after subjecting to column chromatography [2% MeOH in chloroform].

¹H NMR (300 MHz, DMSO-d6) δ 12.73 (s, 1H), 8.30-8.23 (d, J=19.5 Hz, 2H), 7.95-7.93 (d, J=6.9 Hz, 2H), 7.54 (m, 2H), 7.25-7.04 (m, 3H), 6.89 (m, 2H), 6.33 (bs, 1H), 4.25 (s, 2H), 3.95-3.80 (m, 5H), 3.64 (m, 4H), 1.33 (m, 3H).

### Compound-27: (S)-7-((1-(tert-butylcarbamoyl)piperidin-4-yl)amino)-5-chloro-3-((2-(phenoxymethyl)morpholino)sulfonyl)-1H-indole-2-carboxamide

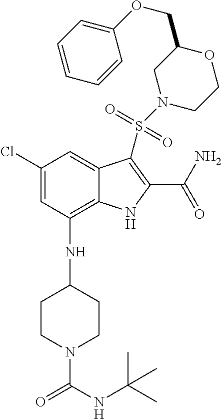 [SMILES: CC(C)(C)NC(=O)N1CCC(Nc2cc(Cl)cc3c(S(=O)(=O)N4CCO[C@H](COc5ccccc5)C4)c(C(N)=O)[nH]c23)CC1]

### Procedure:

Following the procedure described for compound 20 and replacing dihydro-2H-pyran-4(3H)-one with N-(tert-butyl)-4-oxopiperidine-1-carboxamide (1.5 eq), the title compound was obtained after subjecting to reverse phase C18 flash column chromatography [50 to 30% water in acetonitrile].

¹H NMR (300 MHz, DMSO-d6) δ 12.67 (s, 1H), 8.31-8.26 (d, J=12.9 Hz, 2H), 7.28-7.23 (m, 2H), 7.13-7.12 (s, 1H), 6.95-6.87 (m, 3H), 6.46 (s 1H), 6.36-6.34 (d, 1H, J=6.0 Hz), 5.81 (s, 1H), 4.01-3.85 (m, 6H), 3.67-3.59 (m, 2H), 3.52-3.46 (m, 2H), 2.92-2.84 (t, 2H), 2.44-2.30 (m, 2H), 1.95-1.92 (d, 2H), 1.31 (m, 2H), 1.26 (s, 9H).

### Compound-28: (S)-5-chloro-7-((1-(cyclohexylcarbamoyl)piperidin-4-yl)amino)-3-(2-(phenoxymethyl)morpholino)sulfonyl)-1H-indole-2-carboxamide

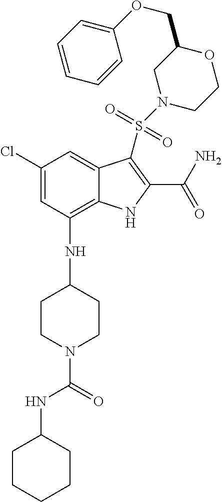 [SMILES: NC(=O)c1[nH]c2c(NC3CCN(C(=O)NC4CCCCC4)CC3)cc(Cl)cc2c1S(=O)(=O)N1CCO[C@H](COc2ccccc2)C1]

### Procedure:

Following the procedure described in compound 20 and replacing dihydro-2H-pyran-4(3H)-one with N-cyclohexyl-4-oxopiperidine-1-carboxamide (2 eq), the title compound was obtained after subjecting to reverse phase C18 flash column chromatography [50 to 30% water in acetonitrile].

¹H NMR (300 MHz DMSO-d6) δ 12.63 (s, 1H), 8.31-8.25 (d, J=15.0 Hz, 2H), 7.28-7.23 (m, 2H), 7.13 (s, 1H), 6.95-6.87 (m, 3H), 6.46 (s, 1H), 6.35-6.33 (d, J=6.0 Hz, 1H), 6.19-6.16 (d, J=9.0 Hz, 1H), 3.95-3.89 (m, 6H), 3.67-3.39 (m, 5H), 2.95-2.87 (t, 2H), 2.41-2.34 (m, 2H), 1.95-1.92 (d, 2H), 1.76-1.72 (t, 4H), 130-1.14 (m, 8H).

### Compound-29: (S)-5-chloro-7-((1-((cyclohexylmethyl)carbamoyl) piperidin-4-yl)amino)-3-((2-(phenoxymethyl)morpholino)sulfonyl)-1H-indole-2-carboxamide

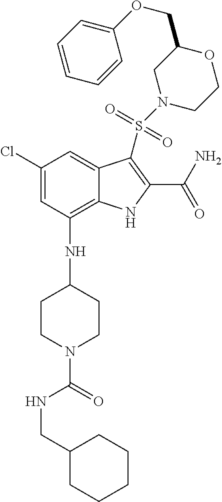 [SMILES: NC(=O)c1[nH]c2c(NC3CCN(C(=O)NCC4CCCCC4)CC3)cc(Cl)cc2c1S(=O)(=O)N1CCO[C@H](COc2ccccc2)C1]

### Procedure:

Following the procedure described in compound 20 and replacing dihydro-2H-pyran-4(3H)-one with N-(cyclohexylmethyl)-4-oxopiperidine-1-carboxamide (2 eq), the title compound was obtained after subjecting to Reverse phase C18 flash column chromatography [50 to 30% water in acetonitrile].

¹H NMR (300 MHz DMSO-d6) δ 12.6 (s, 1H), 8.30-8.21 (d, J=27.0 Hz, 2H), 7.27-7.21 (t, J=9.0 Hz, 2H), 7.12 (s, 1H), 6.93-6.85 (m, 3H), 6.50-6.46 (m, 2H), 6.34-6.32 (d, 1H), 3.92-3.78 (m, 9H), 2.40-2.34 (m, 2H), 1.95-1.92 (d, J=9.0 Hz, 2H), 1.66-1.63 (m, 6H), 1.32-1.13 (m, 12H).

### Compound-31: (S)-5-chloro-7-((1-isobutylpiperidin-4-yl)amino)-3-((2-(phenoxymethyl)morpholino)sulfonyl)-1H-indole-2-carboxamide

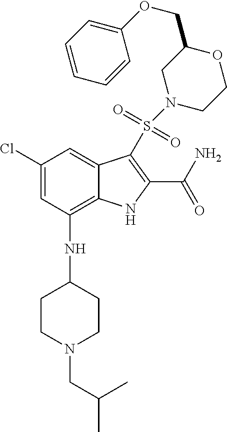 [SMILES: CC(C)CN1CCC(Nc2cc(Cl)cc3c(S(=O)(=O)N4CCO[C@H](COc5ccccc5)C4)c(C(N)=O)[nH]c23)CC1]

Following the procedure described for compound 20, and replacing dihydro-2H-pyran-4(3H)-one with 1-isobutylpiperidin-4-one (1.5 eq), the title compound was obtained after subjecting to column chromatography [2% MeOH in chloroform].

¹H NMR (300 MHz, DMSO-d6) δ 12.65 (s, 1H), 8.29-8.20 (d, J=16.3 Hz, 2H), 7.26-7.21 (m, 2H), 7.10-7.097 (d, J=1.5 Hz, 2H), 6.93-6.85 (m, 3H), 6.35-6.32 (m, 2H), 199-3.88 (m, 3H), 3.78 (m, 2H), 3.66-3.44 (m, 5H), 3.38 (m, 2H), 3.08-3.00 (m, 4H), 2.79-2.76 (m, 2H), 2.54 (m, 1H), 2.39 (m, 1H), 0.86-0.79 (m, 6H).

### Compound-32: 5-chloro-3-(((S)-2-(phenoxymethyl)morpholino)sulfonyl)-7-(pyrrolidin-3-ylamino)-1H-indole-2-carboxamide

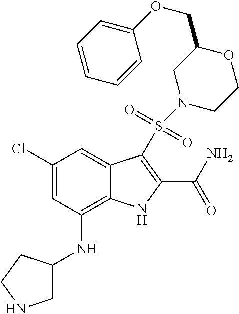 [SMILES: NC(=O)c1[nH]c2c(NC3CCNC3)cc(Cl)cc2c1S(=O)(=O)N1CCO[C@H](COc2ccccc2)C1]

Following the procedure described in compound 20, and replacing dihydro-2H-pyran-4(3H)-one with pyrrolidin-3-one (1.5 eq), the title compound was obtained after subjecting to column chromatography [2% MeOH in chloroform].

¹H NMR (300 MHz, DMSO-d6) δ 12.60 (s, 1H), 8.85-8.81 (m, 2H), 8.30 (s, 1H), 8.21-8.18 (d, J=7.8 Hz, 2H), 7.27-7.22 (m, 3H), 6.97-6.85 (m, 3H), 6.56 (m, 1H), 6.43 (s, 1H), 4.27 (m, 1H), 3.94-3.78 (m, 4H), 3.72-3.44 (m, 3H), 3.16 (m, 1H), 2.35-2.08 (m, 4H), 2.01-1.97 (m, 2H).

### Compound-33: (S)-ethyl 4-(2-carbamoyl-5-fluoro-3-(2-(phenoxymethyl)morpholinosulfonyl)-1H-indol-7-ylamino)piperidine-1-carboxylate

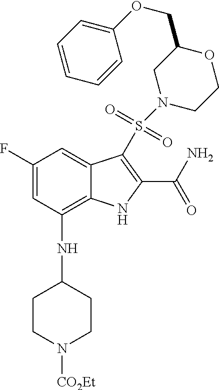 [SMILES: CCOC(=O)N1CCC(Nc2cc(F)cc3c(S(=O)(=O)N4CCO[C@H](COc5ccccc5)C4)c(C(N)=O)[nH]c23)CC1]

### Procedure:

(S)-7-amino-5-fluoro-3-(2-(phenoxymethyl)morpholinosulfonyl)-1H-indole-2-carboxamide (0.15 g), ethyl 4-oxopiperidine-1-carboxylate (0.86 mL.), Hunig base (191 mL) and catalytic amount of DMAP were dissolved in DCM (10 mL) and stirred at RT for 6 hours. Subsequently sodium triacetoxyborohydride (0.3 g) was added and stirred at RT for an additional 14 h. DCM was evaporated in vacuo and the residual solid dissolved in ethyl acetate (25 mL). The organic layer was washed with water (25 mL×2), brine (25 mL×2), dried over Na2SO4 (1 g) and subjected to column chromatography (0.5 to 1.5% methanol/chloroform) to yield the titled compound (0.045 g).

¹H NMR (300 MHz, DMSO-d6) δ 12.60 (s, 1H), 8.32-8.23 (d, J=27.0 Hz, 2H), 7.28-7.23 (m, 2H), 6.95-6.84 (m, 3H), 6.84-6.80 (m, 1H), 6.45-6.34 (m, 2H), 4.09-4.00 (m, 2H), 3.96-3.93 (m, 3H), 3.90 (m, 2H), 3.82-3.79 (m, 1H), 3.68-3.59 (m, 1H), 3.51 (m, 2H), 3.06 (m, 2H), 2.43-2.28 (m, 3H), 2.03-1.99 (m, 2H), 1.23-1.14 (m, 5H).

### Compound 35 (S)-5-chloro-3-((2-(phenoxymethyl)morpholino)sulfonyl)-7-(3-phenylthioureido)-1H-indole-2-carboxamide

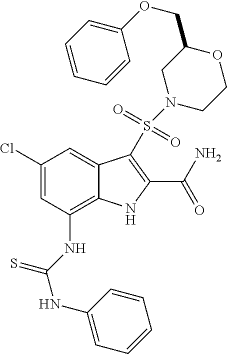 [SMILES: NC(=O)c1[nH]c2c(NC(=S)Nc3ccccc3)cc(Cl)cc2c1S(=O)(=O)N1CCO[C@H](COc2ccccc2)C1]

### Procedure:

Compound 7 and isothiocyanatobenzene (2 eq) were added together in dry THF and stirred in a reaction mixture for 12 hours. The solid was filtered and washed with n hexane to obtain the pure title compound.

¹H NMR (300 MHz DMSO-d6) δ 12.95 (s, 1H), 10.18 (s, 1H), 9.63 (s, 1H), 8.23-8.19 (d, J=12.0 Hz, 2H), 7.72 (s, 1H), 7.57-7.54 (m, 2H), 7.48 (s, 1H), 7.39-7.34 (m, 2H), 7.29-7.24 (m, 2H), 7.19-7.14 (m, 1H), 6.95-6.89 (m, 3H), 3.98-3.97 (m, 2H), 3.86-3.81 (m, 2H), 3.74-3.70 (m, 2H), 3.63-3.56 (m, 2H), 2.27 (m, 1H).

### Compound-36:—(S)-5-chloro-3-((2-(phenoxymethyl)morpholino)sulfonyl)-7-(piperidin-4-ylamino)-1H-indole-2-carboxamide

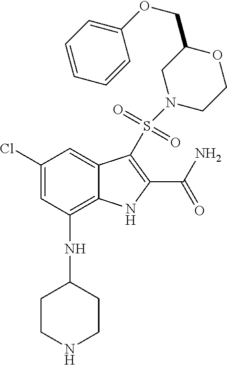 [SMILES: NC(=O)c1[nH]c2c(NC3CCNCC3)cc(Cl)cc2c1S(=O)(=O)N1CCO[C@H](COc2ccccc2)C1]

### Procedure:

The N-Boc protected intermediate of the desired compound was obtained using a similar procedure as described for compound 20. The Boc protected intermediate was dissolved in DCM and subjected to TFA (50% in DCM) treatment for 4 hours to yield the titled compound after purification via column chromatography [0-5% MeOH/Chloroform].

¹H NMR (300 MHz, DMSO-d6) δ 12.61 (s, 1H), 8.50 (bs, 2H), 8.30-8.20 (m, 2H), 7.26-7.17 (m, 2H), 6.99-6.87 (m, 3H), 6.52-6.43 (m, 2H), 3.95-3.90 (m, 3H), 3.79 (m, 1H), 3.68-3.50 (m, 4H), 3.08 (m, 2H), 2.40-2.33 (m, 2H), 2.17-2.14 (m, 2H), 1.63-1.59 (m, 2H), 1.33-1.23 (m, 2H).

Other compounds of the invention can be synthesized using similar procedures as outlined above.

TABLE 1
Representative Compounds

| Sr | | | | IGFIR- |
|---|---|---|---|---|
| No. | Name | Structure | M + H | Activity (nM) |
| 8 | (S)-4-(2-carbamoyl-5-chloro-3-(2- (phenoxymethyl)morpholinosulfonyl)- 1H-indol-7-ylamino)-4-oxobutanoic acid | 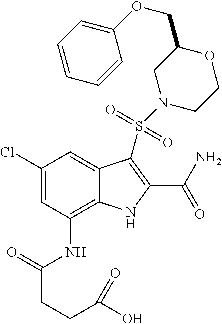 [SMILES: NC(=O)c1[nH]c2c(NC(=O)CCC(=O)O)cc(Cl)cc2c1S(=O)(=O)N1CCO[C@H](COc2ccccc2)C1] | 565.1 | 6.6 |
| 9 | (S)-5-(2-carbamoyl-5-chloro-3-(2- (phenoxymethyl)morpholinosulfonyl)- 1H-indol-7-ylamino)-3,3-dimethyl-5- oxopentanoic acid | 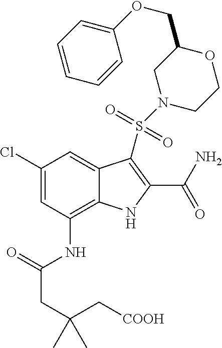 [SMILES: CC(C)(CC(=O)O)CC(=O)Nc1cc(Cl)cc2c(S(=O)(=O)N3CCO[C@H](COc4ccccc4)C3)c(C(N)=O)[nH]c12] | 607.2 | 55.8 |
| 10 | (S)-4-(2-carbamoyl-5-chloro-3-(2- (phenoxymethyl)morpholinosulfonyl)- 1H-indol-7-ylamino)-2,2-dimethyl-4- oxobutanoic acid | 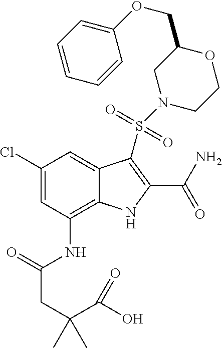 [SMILES: CC(C)(CC(=O)Nc1cc(Cl)cc2c(S(=O)(=O)N3CCO[C@H](COc4ccccc4)C3)c(C(N)=O)[nH]c12)C(=O)O] | 593.1 | 29.9 |
| 11 | (S)-5-(2-carbamoyl-5-chloro-3-(2- (phenoxymethyl)morpholinosulfonyl)- 1H-indol-7-ylamino)-5-oxopentanoic acid | 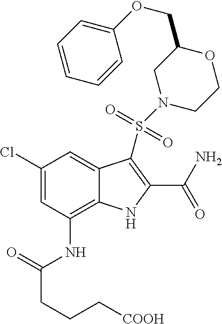 [SMILES: NC(=O)c1[nH]c2c(NC(=O)CCCC(=O)O)cc(Cl)cc2c1S(=O)(=O)N1CCO[C@H](COc2ccccc2)C1] | 579 | 10 |
| 12 | 2-(2-carbamoyl-5-chloro-3-((S)-2- (phenoxymethyl)morpholinosulfonyl)- 1H-indol-7- ylcarbamoyl)cyclopropanecarboxylic acid | 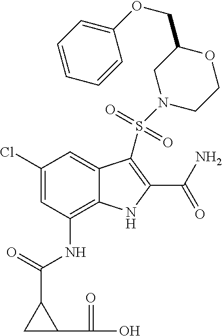 [SMILES: NC(=O)c1[nH]c2c(NC(=O)C3CC3C(=O)O)cc(Cl)cc2c1S(=O)(=O)N1CCO[C@H](COc2ccccc2)C1] | 577.1 | ND |
| 13 | (S)-5-chloro-7-(5-morpholino-5- oxopentanamido)-3-(2- (phenoxymethyl)morpholinosulfonyl)- 1H-indole-2-carboxamide | 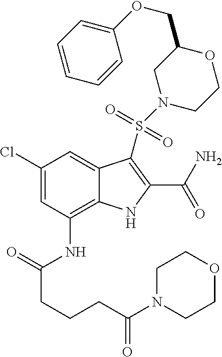 [SMILES: NC(=O)c1[nH]c2c(NC(=O)CCCC(=O)N3CCOCC3)cc(Cl)cc2c1S(=O)(=O)N1CCO[C@H](COc2ccccc2)C1] | 648 | 7.6 |
| 14 | (S)-5-chloro-7-(2-cyanoacetamido)-3- (2- (phenoxymethyl)morpholinosulfonyl)- 1H-indole-2-carboxamide | 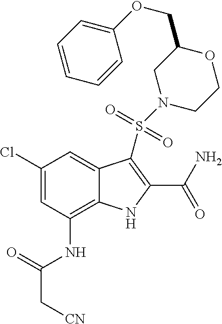 [SMILES: N#CCC(=O)Nc1cc(Cl)cc2c(S(=O)(=O)N3CCO[C@H](COc4ccccc4)C3)c(C(N)=O)[nH]c12] | 532 | >100 |
| 15 | (S)-ethyl 5-(2-carbamoyl-5-chloro-3-(2- (phenoxymethyl)morpholinosulfonyl)- 1H-indol-7-ylamino)-5-oxopentanoate | 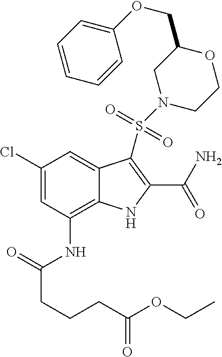 [SMILES: CCOC(=O)CCCC(=O)Nc1cc(Cl)cc2c(S(=O)(=O)N3CCO[C@H](COc4ccccc4)C3)c(C(N)=O)[nH]c12] | 607 | <100 |
| 16 | (S)-3-(2-carbamoyl-5-chloro-3-(2- (phenoxymethyl)morpholinosulfonyl)- 1H-indol-7-ylamino)propanoic acid | 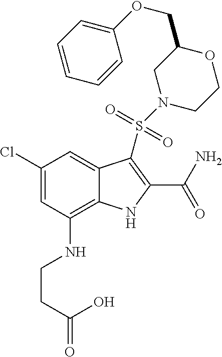 [SMILES: NC(=O)c1[nH]c2c(NCCC(=O)O)cc(Cl)cc2c1S(=O)(=O)N1CCO[C@H](COc2ccccc2)C1] | 537.1 | 2.6 |
| 17 | (S)-7-(3-amino-3-oxopropylamino)-5- chloro-3-(2- (phenoxymethyl)morpholinosulfonyl)- 1H-indole-2-carboxamide | 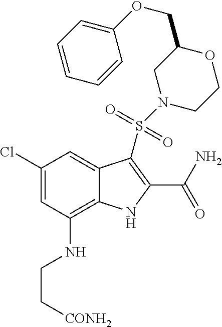 [SMILES: NC(=O)CCNc1cc(Cl)cc2c(S(=O)(=O)N3CCO[C@H](COc4ccccc4)C3)c(C(N)=O)[nH]c12] | 536 | 11.5 |
| 18 | (S)-ethyl 4-(2-carbamoyl-5-chloro-3-(2- (phenoxymethyl)morpholinosulfonyl)- 1H-indol-7-ylamino)butanoate | 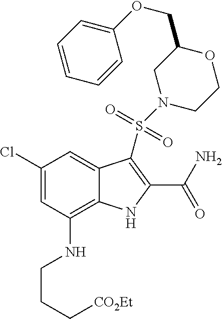 [SMILES: CCOC(=O)CCCNc1cc(Cl)cc2c(S(=O)(=O)N3CCO[C@H](COc4ccccc4)C3)c(C(N)=O)[nH]c12] | 579.2 | >100 |
| 19 | (S)-5-chloro-7-(2-cyanoethylamino)-3- (2- (phenoxymethyl)morpholinosulfonyl)- 1H-indole-2-carboxamide | 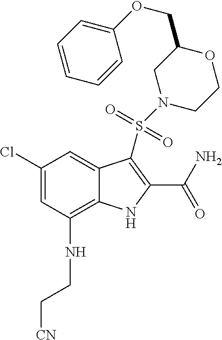 [SMILES: N#CCCNc1cc(Cl)cc2c(S(=O)(=O)N3CCO[C@H](COc4ccccc4)C3)c(C(N)=O)[nH]c12] | 518.1 | 16 |
| 20 | (S)-5-chloro-3-(2- (phenoxymethyl)morpholinosulfonyl)- 7-(tetrahydro-2H-pyran-4-ylamino)-1H- indole-2-carboxamide | 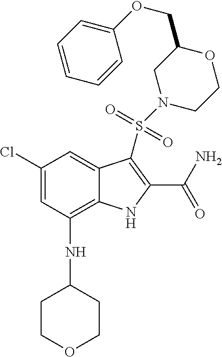 [SMILES: NC(=O)c1[nH]c2c(NC3CCOCC3)cc(Cl)cc2c1S(=O)(=O)N1CCO[C@H](COc2ccccc2)C1] | 549.1 | <100 |
| 21 | (S)-5-chloro-7-(cyclohexylamino)-3-(2- (phenoxymethyl)morpholinosulfonyl)- 1H-indole-2-carboxamide | 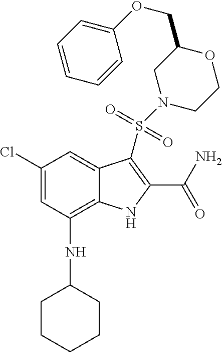 [SMILES: NC(=O)c1[nH]c2c(NC3CCCCC3)cc(Cl)cc2c1S(=O)(=O)N1CCO[C@H](COc2ccccc2)C1] | 547.2 | >100 |
| 22 | (S)-5-chloro-7- (cyclohexylmethylamino)-3-(2- (phenoxymethyl)morpholinosulfonyl)- 1H-indole-2-carboxamide | 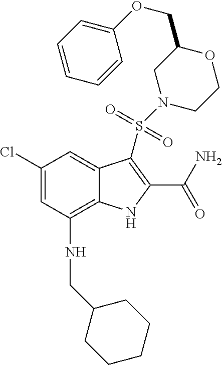 [SMILES: NC(=O)c1[nH]c2c(NCC3CCCCC3)cc(Cl)cc2c1S(=O)(=O)N1CCO[C@H](COc2ccccc2)C1] | 561.2 | >100 |
| 23 | (S)-methyl 4-((2-carbamoyl-5-chloro-3- (2- (phenoxymethyl)morpholinosulfonyl)- 1H-indol-7-ylamino)methyl)benzoate | 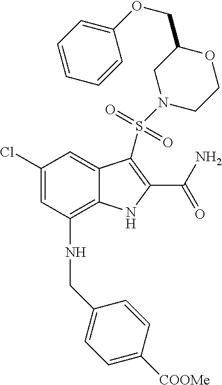 [SMILES: COC(=O)c1ccc(CNc2cc(Cl)cc3c(S(=O)(=O)N4CCO[C@H](COc5ccccc5)C4)c(C(N)=O)[nH]c23)cc1] | 613.1 | |
| 24 | (S)-5-chloro-7-(cyclopentylamino)-3- (2- (phenoxymethyl)morpholinosulfonyl)- 1H-indole-2-carboxamide | 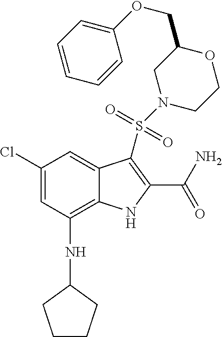 [SMILES: NC(=O)c1[nH]c2c(NC3CCCC3)cc(Cl)cc2c1S(=O)(=O)N1CCO[C@H](COc2ccccc2)C1] | 533.2 | >100 |
| 25 | (S)-7-((1- aminocyclopentyl)methylamino)-5- chloro-3-(2- (phenoxymethyl)morpholinosulfonyl)- 1H-indole-2-carboxamide | 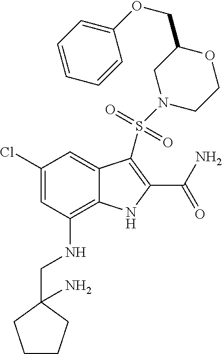 [SMILES: NC(=O)c1[nH]c2c(NCC3(N)CCCC3)cc(Cl)cc2c1S(=O)(=O)N1CCO[C@H](COc2ccccc2)C1] | 562.1 | >100 |
| 26 | (S)-4-((2-carbamoyl-5-chloro-3-(2- (phenoxymethyl)morpholinosulfonyl)- 1H-indol-7-ylamino)methyl)benzoic acid | 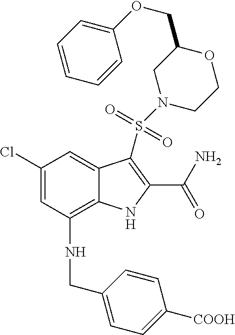 [SMILES: NC(=O)c1[nH]c2c(NCc3ccc(C(=O)O)cc3)cc(Cl)cc2c1S(=O)(=O)N1CCO[C@H](COc2ccccc2)C1] | 599.2 | <100 |
| 27 | (S)-7-(1-tert- butylcarbamoyl)piperidin-4-ylamino)- 5-chloro-3-(2- (phenoxymethyl)morpholinosulfonyl)- 1H-indole-2-carboxamide | 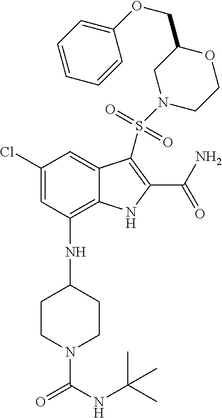 [SMILES: CC(C)(C)NC(=O)N1CCC(Nc2cc(Cl)cc3c(S(=O)(=O)N4CCO[C@H](COc5ccccc5)C4)c(C(N)=O)[nH]c23)CC1] | 647.3 | 9.8 |
| 28 | (S)-5-chloro-7-(1- (cyclohexylcarbamoyl)piperidin-4- ylamino)-3-(2- (phenoxymethyl)morpholinosulfonyl)- 1H-indole-2-carboxamide | 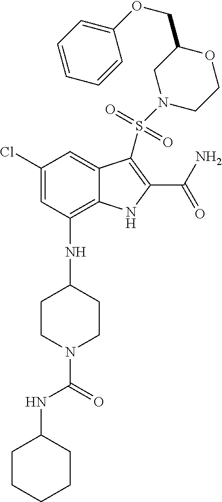 [SMILES: NC(=O)c1[nH]c2c(NC3CCN(C(=O)NC4CCCCC4)CC3)cc(Cl)cc2c1S(=O)(=O)N1CCO[C@H](COc2ccccc2)C1] | 673.2 | 7.7 |
| 29 | (S)-5-chloro-7-(1- (cyclohexylmethylcarbamoyl)piperidin- 4-ylamino)-3-(2- (phenoxymethyl)morpholinosulfonyl)- 1H-indole-2-carboxamide | 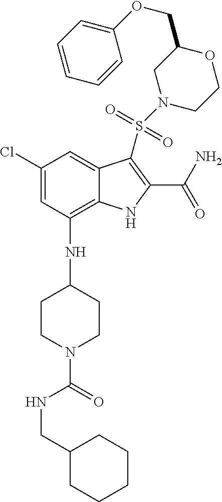 [SMILES: NC(=O)c1[nH]c2c(NC3CCN(C(=O)NCC4CCCCC4)CC3)cc(Cl)cc2c1S(=O)(=O)N1CCO[C@H](COc2ccccc2)C1] | 687.3 | 32.9 |
| 30 | (S)-5-chloro-7-(4-fluorobenzylamino)- 3-(2- (phenoxymethyl)morpholinosulfonyl)- 1H-indole-2-carboxamide | 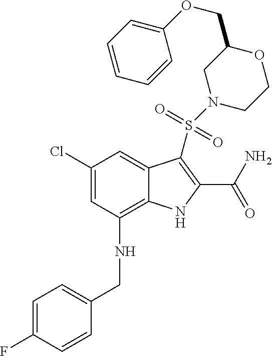 [SMILES: NC(=O)c1[nH]c2c(NCc3ccc(F)cc3)cc(Cl)cc2c1S(=O)(=O)N1CCO[C@H](COc2ccccc2)C1] | 573.1 | >100 |
| 31 | (S)-5-chloro-7-(1-isobutylpiperidin-4- ylamino)-3-(2- (phenoxymethyl)morpholinosulfonyl)- 1H-indole-2-carboxamide | 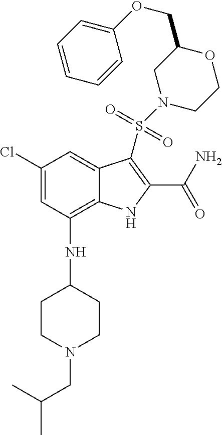 [SMILES: CC(C)CN1CCC(Nc2cc(Cl)cc3c(S(=O)(=O)N4CCO[C@H](COc5ccccc5)C4)c(C(N)=O)[nH]c23)CC1] | 604.3 | 39 |
| 32 | 5-chloro-3-((S)-2- (phenoxymethyl)morpholinosulfonyl)- 7-(pyrrolidin-3-ylamino)-1H-indole-2- carboxamide | 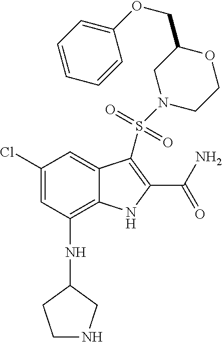 [SMILES: NC(=O)c1[nH]c2c(NC3CCNC3)cc(Cl)cc2c1S(=O)(=O)N1CCO[C@H](COc2ccccc2)C1] | 534.1 | 48.7 |
| 33 | (S)-ethyl 4-(2-carbamoyl-5-fluoro-3-(2- (phenoxymethyl)morpholinosulfonyl)- 1H-indol-7-ylamino)piperidine-1- carboxylate | 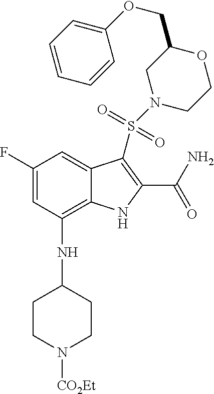 [SMILES: CCOC(=O)N1CCC(Nc2cc(F)cc3c(S(=O)(=O)N4CCO[C@H](COc5ccccc5)C4)c(C(N)=O)[nH]c23)CC1] | 604.2 | <100 |
| 34 | (S)-ethyl 4-(2-carbamoyl-5-chloro-3-(2- (phenoxymethyl)morpholinosulfonyl)- 1H-indol-7-ylamino)piperidine-1- carboxylate | 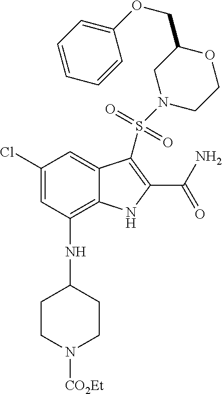 [SMILES: CCOC(=O)N1CCC(Nc2cc(Cl)cc3c(S(=O)(=O)N4CCO[C@H](COc5ccccc5)C4)c(C(N)=O)[nH]c23)CC1] | 620.2 | 68.5 |
| 35 | (S)-5-chloro-3-(2- (phenoxymethyl)morpholinosulfonyl)- 7-(3-phenylthioureido)-1H-indole-2- carboxamide | 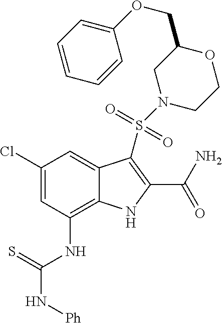 [SMILES: NC(=O)c1[nH]c2c(NC(=S)Nc3ccccc3)cc(Cl)cc2c1S(=O)(=O)N1CCO[C@H](COc2ccccc2)C1] | 600.1 | 1.3 |
| 36 | (S)-5-chloro-3-(2- (phenoxymethyl)morpholinosulfonyl)- 7-(piperidin-4-ylamino)-1H-indole-2- carboxamide | 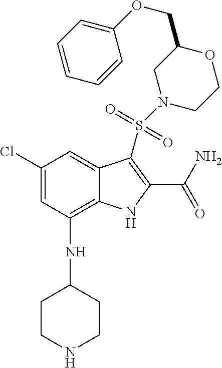 [SMILES: NC(=O)c1[nH]c2c(NC3CCNCC3)cc(Cl)cc2c1S(=O)(=O)N1CCO[C@H](COc2ccccc2)C1] | 548 | 22 |

### Example 2

### In Vitro IGF-1R and IR Kinase Assays

The in vitro kinase assays using IGF-1R and IR kinase GST fusion proteins were conducted using a homogeneous time-resolved fluorescence (HTRF) format. Kinase reactions were carried out in a 384-well plate format in a final volume of 204. The standard enzyme reaction buffer consisted of 50 mM Tris HCL (pH: 7.4), 1 mM EGTA, 10 mM MgCl₂, 2 mM DTT, 0.01% Tween-20, IGF-1R/IR kinase enzyme, poly GT peptide substrate (Perkin Elmer [Ulight Glu-Tyr (4:1)]n) and ATP [concentration equivalent to Kmₐₚₚ]. Inhibitors in DMSO (<1%), were added to give a final inhibitor concentration ranging from 40 μM to 40 μM. Briefly, 2.5 μL enzyme and 2.5 μL inhibitor was pre-incubated for 10 minutes at 23° C. followed by the addition of 2.5 μL of poly GT substrate (final concentration of 50 nM). Reaction was initiated with the addition of 2.5 μL of ATP (final concentration of 20 μM for IGF-1R assay and 10 μM for IR assay). After 1 hour incubation at 23° C., the kinase reaction was stopped with the addition of 5 μL EDTA (final concentration of 10 mM in 20 μL). Europium cryptate—labeled antiphosphotyrosine antibody PY20 (5 μL) was added (final concentration of 2 nM) and the mixture was allowed to equilibrate for 1 hour at 23° C. followed by reading the plate in an Envision plate reader. The intensity of light emission at 665 nm was directly proportional to the level of substrate phosphorylation. The IC₅₀ values for inhibitors were determined by a four-parameter sigmoidal curve fit (Sigma plot or Graph pad).

IGFRK and IRK enzyme used for the assay was intracellular kinase domain of human IGF-1R and human IR cloned and expressed as GST fusion proteins using the baculovirus expression system and purified using glutathione—Sepharose column. IGFRK was used at a final concentration of 0.25 nM and IRK at 0.5 nM.

### Example 3

### Anti-Proliferative Assay

Anti-proliferative potential of compounds was tested using various cell lines (details provided in Table 2) by MTS (Promega, Cat # G1111), a tetrazolium compound ((3-(4,5-dimethylthiazol-2-yl)-5-(3-carboxymethoxyphenyl)-2-(4-sulphophenyl)-2H-tetrazolium, inner salt; MTS) and Cell Counting kit-8 (CCK-8 a Dojindo's highly water-soluble tetrazolium salt of WST-8 [2-(2-methoxy-4-nitrophenyl)-3-(4-nitrophenyl)-5-(2,4-disulfophenyl)-2H-tetrazolium, monosodium salt]). MTS is a colorimetric assay for determining the number of viable cells in proliferation, cytotoxicity or chemosensitivity assays. This is used with an electron coupling reagent PMS (Phenazine methosulfate). MTS is bioreduced by cells into a formazan that is soluble in tissue culture medium. The absorbance of the formazan at 490 nm can be measured directly from 96 well assay plates without additional processing. Dehydrogenase enzymes found in metabolically active cells accomplish the conversion of MTS into the aqueous soluble formazan. The quantity of formazan product is directly proportional to the number of living cells in culture. In CCK-8, WST-8 is reduced by dehydrogenases in cells to give a yellow colored product formazan, which is measured at 450 nm.

For experimental purposes, cells were seeded at a density of 3000-5000 cells per well in 180 μL/well volume in transparent 96 well tissue culture plate (NUNC, USA) and incubated overnight at 37° C., 5% CO₂. Next day before adding compound the medium was replaced and 180 μL of fresh medium added with the 100 ng/mL IGF without FCS followed by addition of 20 μL of 10× compound (10 mM stock made in DMSO and then further dilutions were made in medium, final DMSO concentration should not exceed 0.5%) and incubated for 72 hours in humidified 5% CO₂ incubator at 37±1° C. After incubation medium was replaced with 200 μL of medium containing 20 μL MTS reagent per well. Plates were incubated for 3-4 hours and absorbance was measured at 490 nm on Spectrophotometer (SpectraMax, Molecular Devices). Percentage cytotoxicity and IC₅₀ was calculated using SoftMax software. CCK-8 was used for suspension cell lines. Cell seeding and compound addition was done on same day. Following the incubation, 10 μL of CCK-8 solution was added in each well. After 4 hour incubation, the absorbance was determined at 450 nm using Spectrophotometer (SpectraMax, Molecular Devices). In every experiment, each condition was run in triplicate wells.

TABLE 2
Anti-proliferation IC₅₀ (μM)

| Compound | MCF7 cell line | HT29 cell line |
|---|---|---|
| 100 | 2.2 | >10 |
| 19 | <1 | 5.3 |
| 20 | 0.8 | 2.8 |
| 34 | 0.9 | 1.7 |

Compound 100

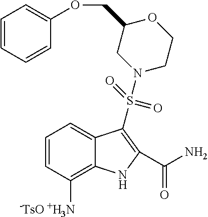

As shown in Table 2, compared to Compound 100, Compounds 19, 20 and 34 showed higher anti-proliferation activity in colon cancer and breast cancer cell lines.

### Example 4

### CYP Inhibition Fluorescence Assay

The % inhibition @ 10 μM data was generated from an rhCYP450/fluorescence assay according to the Vivid Invitrogen screening kits. The compounds were screened against CYP 3A4 isoform because CYP3A4 is responsible for the metabolism of approximately 50-60% of clinical drugs. The lower the percentage inhibition, the lower the CYP450 inhibitory liability of that specific compound.

TABLE 3
CYP inhibition data using fluorescence assay

| Compound | % inhibition of CYP 3A4 at 10 μM |
|---|---|
| 8 | 0 |
| 35 | <40 |
| 11 | 15 |
| 13 | 46 |
| 34 | 47 |
| 100 | 98 |

As shown in Table 3, the CYP 3A4 inhibitory liability of compounds 8, 35, 11, 13 and 34 are lower than compound 100.

1. A compound as illustrated by Formula I: 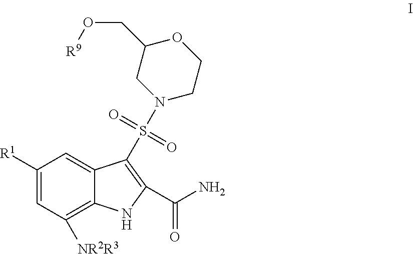
wherein:
R^{a} is independently selected from the group consisting of H and C₁-C₆ alkyl,
said alkyl is optionally substituted with one to three substituents selected from R⁷;
R¹ is selected from the group consisting of:
H,
Halogen,
NO₂,
CN,
(CR^{a}₂)ₙOR⁵,
(CR^{a}₂)ₙN(R⁵)₂,
C(O)R⁵,
C(O)OR⁵,
(CR^{a}₂)ₙR⁵,
S(O)ₘR⁵,
S(O)ₘN(R⁵)₂,
SR⁵,
OS(O)ₘR⁵,
N(R⁵)C(O)R⁵,
N(R⁵)S(O)ₘR⁵, and
(CR^{a}₂)ₙC(O)N(R⁵)₂;
R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to four substituents selected from the group consisting of halogen, C₁-C₆ alkyl, NR⁸C(O)R¹⁰, C(O)NR⁸R¹⁰ and C(O)OR¹²;
R⁵ is independently selected from the group consisting of:
H,
C₆-C₁₀aryl,
5-10 membered heterocyclyl,
5-10 membered heterocyclenyl,
5-10 membered heteroaryl,
C₁-C₆ alkyl, and
C₃-C₈ cycloalkyl,
said aryl, heterocyclyl, heterocyclenyl, heteroaryl, alkyl and cycloalkyl is optionally substituted with one to three substituents selected from R⁷;
R⁷ is independently selected from the group consisting of:
C₁-C₆ alkyl,
Halogen,
C₁-C₆ alkoxy,
C₁-C₆ haloalkyl,
CN,
NH₂, and
NO₂;
R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl,
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl, optionally substituted with one to three substituents selected from R⁷;
R¹² is H or C₁-C₆ alkyl;
X is C₁-C₆ alkylene or C₃-C₈cycloalkylene;
Y is selected from the group consisting of H, OR¹², CN, heterocyclyl, NR⁸R¹⁰, NH₂, C₃-C₈cycloalkyl, wherein C₃-C₈cycloalkyl is optionally substituted with one to three substituents selected from the group consisting of halogen, C₁-C₆ alkyl, C(O)NR⁸R¹⁰, C(O)OR¹² and NR⁸R¹¹, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;
Z is NH, O or S;
m is 1 or 2;
n is independently 0, 1, 2, 3, 4, 5 or 6;
or a pharmaceutically acceptable salt thereof.

2. The compound of claim 1, wherein
R^{a} is independently selected from the group consisting of H and C₁-C₆ alkyl,
said alkyl is optionally substituted with one to three substituents selected from R⁷;
R¹ is selected from the group consisting of:
H,
Halogen,
NO₂,
CN,
(CR^{a}₂)ₙOR⁵,
(CR^{a}₂)ₙN(R⁵)₂,
C(O)R⁵,
C(O)OR⁵,
(CR^{a}₂)ₙR⁵,
S(O)ₘR⁵,
S(O)ₘN(R⁵)₂,
SR⁵,
OS(O)ₘR⁵,
N(R⁵)C(O)R⁵,
N(R⁵)S(O)ₘR⁵, and
(CR^{a}₂)ₙC(O)N(R⁵)₂;
R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to four substituents selected from the group consisting of halogen, C₁-C₆ alkyl, NR⁸C(O)R¹⁰, C(O)NR⁸R¹⁰ and C(O)OR¹²;
R⁵ is independently selected from the group consisting of:
H,
C₆-C₁₀aryl,
5-10 membered heterocyclyl,
5-10 membered heterocyclenyl,
5-10 membered heteroaryl,
C₁-C₆ alkyl, and
C₃-C₈ cycloalkyl,
said aryl, heterocyclyl, heterocyclenyl, heteroaryl, alkyl and cycloalkyl is optionally substituted with one to three substituents selected from R⁷;
R⁷ is independently selected from the group consisting of:
C₁-C₆ alkyl,
Halogen,
C₁-C₆ alkoxy,
C₁-C₆ haloalkyl,
CN,
NH₂, and
NO₂;
R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl,
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl, optionally substituted with one to three substituents selected from R⁷;
R¹² is H or C₁-C₆ alkyl;
X is C₂-C₆ alkylene or C₃-C₈cycloalkylene;
Y is selected from the group consisting of H, OR¹², CN, heterocyclyl, NR⁸R¹⁰, NH₂, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;
Z is NH, O or S;
m is 1 or 2;
n is independently 0, 1, 2, 3, 4, 5 or 6,
or a pharmaceutically acceptable salt thereof.

3. The compound of claim 2,
wherein
R¹ is H, halogen, or CN;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of halogen, C₁-C₆ alkyl, NR⁸C(O)R¹⁰, C(O)NR⁸R¹⁰ and C(O)OR¹²;
R⁸ is H or C₁-C₃ alkyl;
R⁹ is selected from the group consisting of C₆-C₁₀aryl and 5-10 membered heteroaryl, said aryl or heteroaryl is optionally substituted with one to three substituents selected from R⁷;
R¹¹ is independently selected from the group consisting of C₆-C₁₀aryl and 5-10 membered heteroaryl, optionally substituted with one to three substituents selected from R⁷;
R¹² is H or C₁-C₃ alkyl;
Z is O or S;
X is C₂-C₅ alkylene, or cyclopropylene;
and all other substituents are as defined in claim 2.

4. The compound of claim 2 under formula IA: 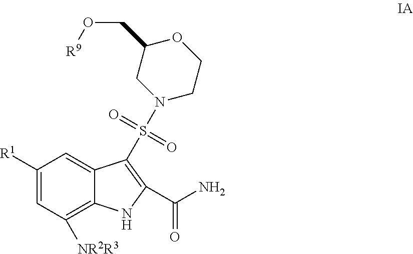
wherein all substituents are as defined in claim 2.

5. The compound of claim 2, wherein
R¹ is halogen;
R² is H;
R³ is —C(O)—X—C(O)—Y, —X—Y, —C(S)—NR¹¹R⁸, or heterocyclyl selected from the group consisting of tetrahydro-pyranyl, piperidinyl and pyrrolidinyl, and wherein the heterocyclyl is optionally substituted with halogen, C(O)NR⁸R¹⁰, C₁-C₆ alkyl, or C(O)OR¹²;
R⁸ is H;
R⁹ is phenyl or pyridyl optionally substituted with one to three substituents selected from R⁷;
R¹¹ is phenyl optionally substituted with one to three substituents selected from R⁷;
R¹² is C₁-C₃ alkyl;
Y is selected from the group consisting of H, OR¹², CN, morpholinyl, and NH₂, wherein said morpholinyl is optionally substituted with C(O)NR⁸R¹⁰, C₁-C₆ alkyl, or C(O)OR¹²;
and all other substituents are as defined in claim 2.

6. The compound of claim 2 under Formula II, 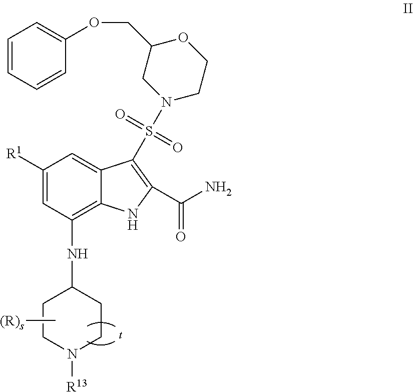
wherein R¹ is halogen;
R¹³ is selected from the group consisting of H, C(O)NR⁸R¹⁰, C₁-C₆ alkyl, and C(O)OR¹²;
R⁸ is H or C₁-C₃ alkyl;
R¹⁰ is selected from the group consisting of C₃-C₈cycloalkyl, C₁-C₆alkyl, and C₃-C₈cycloalkylC₁-C₃alkyl,
R¹² is H or C₁-C₃ alkyl;
R is halogen;
s is 0, 1, 2, 3, or 4;
t is 0 or 1.

7. The compound of claim 6 under Formula IIA: 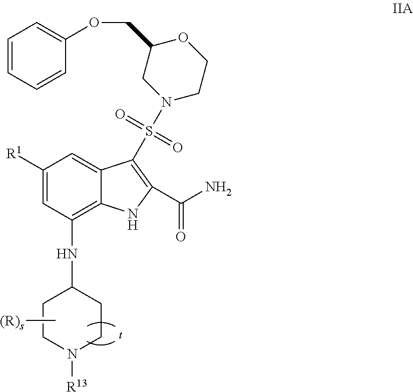
wherein all substituents are as defined in claim 6.

8. The compound of claim 7, wherein
R¹³ is C(O)OR¹²;
R¹² is H or C₁-C₃ alkyl.

9. The compound of claim 2,
wherein:
R^{a} is independently selected from the group consisting of H and C₁-C₆ alkyl,
said alkyl is optionally substituted with one to three substituents selected from R⁷;
R¹ is selected from the group consisting of:
1) H,
2) Halogen,
3) NO₂,
4) CN,
5) (CR^{a}₂)ₙOR⁵,
6) (CR^{a}₂)ₙN(R⁵)₂,
7) C(O)R⁵,
8) C(O)OR⁵,
9) (CR^{a}₂)ₙR⁵,
10) S(O)ₘR⁵,
11) S(O)ₘN(R⁵)₂,
12) SR⁵,
13) OS(O)ₘR⁵,
14) N(R⁵)C(O)R⁵,
15) N(R⁵)S(O)ₘR⁵, and
16) (CR^{a}₂)ₙC(O)N(R⁵)₂;
R² is H or C₁-C₆ alkyl;
R³ is 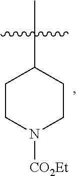 [SMILES: *C1CCN(C(=O)OCC)CC1]
—C(Z)—X—C(O)—Y, or C(S)—NH-Ph;
R⁵ is independently selected from the group consisting of:
1) 11,
2) C₆-C₁₀aryl,
3) 5-10 membered heterocyclyl,
4) 5-10 membered heterocyclenyl,
5) 5-10 membered heteroaryl,
6) C₁-C₆ alkyl, and
7) C₃-C₈ cycloalkyl,
said aryl, heterocyclyl, heterocyclenyl, heteroaryl, alkyl and cycloalkyl is optionally substituted with one to three substituents selected from R⁷;
R⁷ is independently selected from the group consisting of:
1) C₁-C₆ alkyl,
2) Halogen,
3) C₁-C₆ alkoxy,
4) C₁-C₆ haloalkyl,
5) CN,
6) NH₂, and
7) NO₂;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
X is C₂-C₃ alkylene;
Y is OH or morpholinyl;
Z is O or S;
m is 1 or 2;
n is independently 0, 1, 2, 3, 4, 5 or 6;
or a pharmaceutically acceptable salt thereof.

10. A compound selected from the group consisting of:
(S)-4-(2-carbamoyl-5-chloro-3-(2-(phenoxymethyl)morpholinosulfonyl)-1H-indol-7-ylamino)-4-oxobutanoic acid;
(S)-5-(2-carbamoyl-5-chloro-3-(2-(phenoxymethyl)morpholinosulfonyl)-1H-indol-7-ylamino)-3,3-dimethyl-5-oxopentanoic acid;
(S)-4-(2-carbamoyl-5-chloro-3-(2-(phenoxymethyl)morpholinosulfonyl)-1H-indol-7-ylamino)-2,2-dimethyl-4-oxobutanoic acid;
(S)-5-(2-carbamoyl-5-chloro-3-(2-(phenoxymethyl)morpholinosulfonyl)-1,1-indol-7-ylamino)-5-oxopentanoic acid;
2-(2-carbamoyl-5-chloro-3-((S)-2-(phenoxymethyl)morpholinosulfonyl)-1H-indol-7-ylcarbamoyl)cyclopropanecarboxylic acid;
(S)-5-chloro-7-(5-morpholino-5-oxopentanamido)-3-(2-(phenoxymethyl)morpholinosulfonyl)-1H-indole-2-carboxamide;
(S)-5-chloro-7-(2-cyanoacetamido)-3-(2-(phenoxymethyl)morpholinosulfonyl)-1H-indole-2-carboxamide;
(S)-ethyl 5-(2-carbamoyl-5-chloro-3-(2-(phenoxymethyl)morpholinosulfonyl)-1H-indol-7-ylamino)-5-oxopentanoate;
(S)-3-(2-carbamoyl-5-chloro-3-(2-(phenoxymethyl)morpholinosulfonyl)-1H-indol-7-ylamino)propanoic acid;
(S)-7-(3-amino-3-oxopropylamino)-5-chloro-3-(2-(phenoxymethyl)morpholinosulfonyl)-1H-indole-2-carboxamide;
(S)-ethyl 4-(2-carbamoyl-5-chloro-3-(2-(phenoxymethyl)morpholinosulfonyl)-1H-indol-7-ylamino)butanoate;
(S)-5-chloro-7-(2-cyanoethylamino)-3-(2-(phenoxymethyl)morpholinosulfonyl)-1H-indole-2-carboxamide;
(S)-5-chloro-3-(2-(phenoxymethyl)morpholinosulfonyl)-7-(tetrahydro-2H-pyran-4-ylamino)-1H-indole-2-carboxamide;
(S)-5-chloro-7-(cyclohexylamino)-3-(2-(phenoxymethyl)morpholinosulfonyl)-1H-indole-2-carboxamide;
(S)-5-chloro-7-(cyclohexylmethylamino)-3-(2-(phenoxymethyl)morpholinosulfonyl)-1H-indole-2-carboxamide;
(S)-methyl 4-((2-carbamoyl-5-chloro-3-(2-(phenoxymethyl)morpholinosulfonyl)-1H-indol-7-ylamino)methyl)benzoate;
(S)-5-chloro-7-(cyclopentylamino)-3-(2-(phenoxymethyl)morpholinosulfonyl)-1H-indole-2-carboxamide;
(S)-7-((1-aminocyclopentyl)methylamino)-5-chloro-3-(2-(phenoxymethyl)morpholinosulfonyl)-1H-indole-2-carboxamide;
(S)-4-(2-carbamoyl-5-chloro-3-(2-(phenoxymethyl)morpholinosulfonyl)-1H-indol-7-ylamino)methyl)benzoic acid;
(S)-7-(1-(tert-butylcarbamoyl)piperidin-4-ylamino)-5-chloro-3-(2-(phenoxymethyl)morpholinosulfonyl)-1H-indole-2-carboxamide;
(S)-5-chloro-7-(1-(cyclohexylcarbamoyl)piperidin-4-ylamino)-3-(2-(phenoxymethyl)morpholinosulfonyl)-1H-indole-2-carboxamide;
(S)-5-chloro-7-(1-(cyclohexylmethylcarbamoyl)piperidin-4-ylamino)-3-(2-(phenoxymethyl)morpholinosulfonyl)-1H-indole-2-carboxamide;
(S)-5-chloro-7-(4-fluorobenzylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-chloro-3-((S)-2-(phenoxymethyl)morpholinosulfonyl)-7-(pyrrolidin-3-ylamino)-1H-indole-2-carboxamide;
(S)-ethyl 4-(2-carbamoyl-5-fluoro-3-(2-(phenoxymethyl)morpholinosulfonyl)-1H-indol-7-ylamino)piperidine-1-carboxylate;
(S)-ethyl 4-(2-carbamoyl-5-chloro-3-(2-(phenoxymethyl)morpholinosulfonyl)-1H-indol-7-ylamino)piperidine-1-carboxylate;
(S)-5-chloro-3-(2-(phenoxymethyl)morpholinosulfonyl)-7-(3-phenylthioureido)-1H-indole-2-carboxamide; and
(S)-5-chloro-3-(2-(phenoxymethyl)morpholinosulfonyl)-7-(piperidin-4-ylamino)-1H-indole-2-carboxamide;
or a stereoisomer thereof;
or a pharmaceutically acceptable salt thereof;
or a pharmaceutically acceptable salt of the stereoisomer thereof.

11. The compound of claim 2 that is 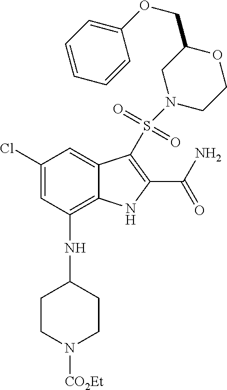 [SMILES: CCOC(=O)N1CCC(Nc2cc(Cl)cc3c(S(=O)(=O)N4CCO[C@H](COc5ccccc5)C4)c(C(N)=O)[nH]c23)CC1]
or a pharmaceutically acceptable salt thereof.

12. The compound of claim 2 that is 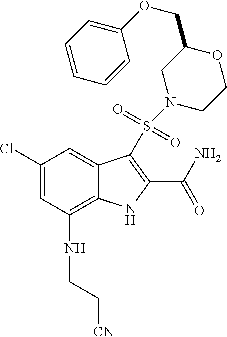 [SMILES: N#CCCNc1cc(Cl)cc2c(S(=O)(=O)N3CCO[C@H](COc4ccccc4)C3)c(C(N)=O)[nH]c12]
or a pharmaceutically acceptable salt thereof.

13. The compound of claim 2 that is 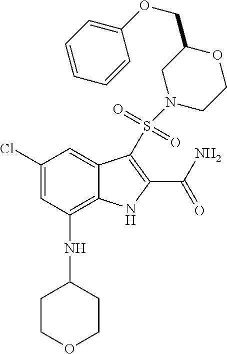 [SMILES: NC(=O)c1[nH]c2c(NC3CCOCC3)cc(Cl)cc2c1S(=O)(=O)N1CCO[C@H](COc2ccccc2)C1]
or a pharmaceutically acceptable salt thereof.

14. A pharmaceutical composition comprising a therapeutically effective amount of the compound of claim 2, and a pharmaceutically acceptable carrier.

15. (canceled)

16. The compound of claim 3 under formula IA: 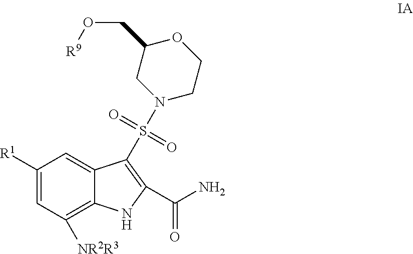
wherein all substituents are as defined in claim 3.

17. The compound of claim 4, wherein
R¹ is halogen;
R² is H;
R³ is —C(O)—X—C(O)—Y, —X—Y, —C(S)—NR¹¹R⁸, or heterocyclyl selected from the group consisting of tetrahydro-pyranyl, piperidinyl and pyrrolidinyl, and wherein the heterocyclyl is optionally substituted with halogen, C(O)NR⁸R¹⁹, C₁-C₆ alkyl, or C(O)OR¹²;
R⁸ is H;
R⁹ is phenyl or pyridyl optionally substituted with one to three substituents selected from R⁷;
R¹¹ is phenyl optionally substituted with one to three substituents selected from R⁷;
R¹² is C₁-C₃ alkyl;
Y is selected from the group consisting of H, OR¹², CN, morpholinyl, and NH₂, wherein said morpholinyl is optionally substituted with C(O)NR⁸R¹⁰, C₁-C₆ alkyl, or C(O)OR¹²;
and all other substituents are as defined in claim 4.

18. The compound of claim 4,
wherein:
R^{a} is independently selected from the group consisting of H and C₁-C₆ alkyl,
said alkyl is optionally substituted with one to three substituents selected from R⁷;
R¹ is selected from the group consisting of:
17) H,
18) Halogen,
19) NO₂,
20) CN,
21) (CR^{a}₂)ₙOR⁵,
22) (CR^{a}₂)ₙN(R⁵)₂,
23) C(O)R⁵,
24) C(O)OR⁵,
25) (CR^{a}₂)ₙR⁵,
26) S(O)ₘR⁵,
27) S(O)ₘN(R⁵)₂,
28) SR⁵,
29) OS(O)ₘR⁵,
30) N(R⁵)C(O)R⁵,
31) N(R⁵)S(O)ₘR⁵, and
32) (CR^{a}₂)ₙC(O)N(R⁵)₂;
R² is H or C₁-C₆ alkyl;
R³ is 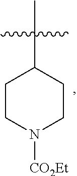 [SMILES: *C1CCN(C(=O)OCC)CC1]
—C(Z)—X—C(O)—Y, or C(S)—NH-Ph;
R⁵ is independently selected from the group consisting of:
8) H,
9) C₆-C₁₀aryl,
10) 5-10 membered heterocyclyl,
11) 5-10 membered heterocyclenyl,
12) 5-10 membered heteroaryl,
13) C₁-C₆ alkyl, and
14) C₃-C₈ cycloalkyl,
said aryl, heterocyclyl, heterocyclenyl, heteroaryl, alkyl and cycloalkyl is optionally substituted with one to three substituents selected from R⁷;
R⁷ is independently selected from the group consisting of:
8) C₁-C₆ alkyl,
9) Halogen,
10) C₁-C₆ alkoxy,
11) C₁-C₆ haloalkyl,
12) CN,
13) NH₂, and
14) NO₂;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
X is C₂-C₃ alkylene;
Y is OH or morpholinyl;
Z is O or S;
m is 1 or 2;
n is independently 0, 1, 2, 3, 4, 5 or 6;
or a pharmaceutically acceptable salt thereof.

19. The composition of claim 14, further comprising an additional therapeutic agent.

20. The composition of claim 14, in which the compound is selected from the group consisting of:
(S)-4-(2-carbamoyl-5-chloro-3-(2-(phenoxymethyl)morpholinosulfonyl)-1H-indol-7-ylamino)-4-oxobutanoic acid;
(S)-5-(2-carbamoyl-5-chloro-3-(2-(phenoxymethyl)morpholinosulfonyl)-1H-indol-7-ylamino)-3,3-dimethyl-5-oxopentanoic acid;
(S)-4-(2-carbamoyl-5-chloro-3-(2-(phenoxymethyl)morpholinosulfonyl)-1H-indol-7-ylamino)-2,2-dimethyl-4-oxobutanoic acid;
(S)-5-(2-carbamoyl-5-chloro-3-(2-(phenoxymethyl)morpholinosulfonyl)-1H-indol-7-ylamino)-5-oxopentanoic acid;
2-(2-carbamoyl-5-chloro-3-((S)-2-(phenoxymethyl)morpholinosulfonyl)-1H-indol-7-ylcarbamoyl)cyclopropanecarboxylic acid;
(S)-5-chloro-7-(5-morpholino-5-oxopentanamido)-3-(2-(phenoxymethyl)morpholinosulfonyl)-1H-indole-2-carboxamide;
(S)-5-chloro-7-(2-cyanoacetamido)-3-(2-(phenoxymethyl)morpholinosulfonyl)-1H-indole-2-carboxamide;
(S)-ethyl 5-(2-carbamoyl-5-chloro-3-(2-(phenoxymethyl)morpholinosulfonyl)-1H-indol-7-ylamino)-5-oxopentanoate;
(S)-3-(2-carbamoyl-5-chloro-3-(2-(phenoxymethyl)morpholinosulfonyl)-1H-indol-7-ylamino)propanoic acid;
(S)-7-(3-amino-3-oxopropylamino)-5-chloro-3-(2-(phenoxymethyl)morpholinosulfonyl)-1H-indole-2-carboxamide;
(S)-ethyl 4-(2-carbamoyl-5-chloro-3-(2-(phenoxymethyl)morpholinosulfonyl)-1H-indol-7-ylamino)butanoate;
(S)-5-chloro-7-(2-cyanoethylamino)-3-(2-(phenoxymethyl)morpholinosulfonyl)-1H-indole-2-carboxamide;
(S)-5-chloro-3-(2-(phenoxymethyl)morpholinosulfonyl)-7-(tetrahydro-2H-pyran-4-ylamino)-1H-indole-2-carboxamide;
(S)-5-chloro-7-(cyclohexylamino)-3-(2-(phenoxymethyl)morpholinosulfonyl)-1H-indole-2-carboxamide;
(S)-5-chloro-7-(cyclohexylmethylamino)-3-(2-(phenoxymethyl)morpholinosulfonyl)-1H-indole-2-carboxamide;
(S)-methyl 4-((2-carbamoyl-5-chloro-3-(2-(phenoxymethyl)morpholinosulfonyl)-1H-indol-7-ylamino)methyl)benzoate;
(S)-5-chloro-7-(cyclopentylamino)-3-(2-(phenoxymethyl)morpholinosulfonyl)-1H-indole-2-carboxamide;
(S)-7-((1-aminocyclopentyl)methylamino)-5-chloro-3-(2-(phenoxymethyl)morpholinosulfonyl)-1H-indole-2-carboxamide;
(S)-4-((2-carbamoyl-5-chloro-3-(2-(phenoxymethyl)morpholinosulfonyl)-1H-indol-7-ylamino)methyl)benzoic acid;
(S)-7-(1-(tert-butylcarbamoyl)piperidin-4-ylamino)-5-chloro-3-(2-(phenoxymethyl)morpholinosulfonyl)-1H-indole-2-carboxamide;
(S)-5-chloro-7-(1-(cyclohexylcarbamoyl)piperidin-4-ylamino)-3-(2-(phenoxymethyl)morpholinosulfonyl)-1H-indole-2-carboxamide;
(S)-5-chloro-7-(1-(cyclohexylmethylcarbamoyl)piperidin-4-ylamino)-3-(2-(phenoxymethyl)morpholinosulfonyl)-1H-indole-2-carboxamide;
(S)-5-chloro-7-(4-fluorobenzylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-chloro-3-((S)-2-(phenoxymethyl)morpholinosulfonyl)-7-(pyrrolidin-3-ylamino)-1H-indole-2-carboxamide;
(S)-ethyl 4-(2-carbamoyl-5-fluoro-3-(2-(phenoxymethyl)morpholinosulfonyl)-1H-indol-7-ylamino)piperidine-1-carboxylate;
(S)-ethyl 4-(2-carbamoyl-5-chloro-3-(2-(phenoxymethyl)morpholinosulfonyl)-1H-indol-7-ylamino)piperidine-1-carboxylate;
(S)-5-chloro-3-(2-(phenoxymethyl)morpholinosulfonyl)-7-(3-phenylthioureido)-1H-indole-2-carboxamide; and
(S)-5-chloro-3-(2-(phenoxymethyl)morpholinosulfonyl)-7-(piperidin-4-ylamino)-1H-indole-2-carboxamide;
or a stereoisomer thereof;
or a pharmaceutically acceptable salt thereof;
or a pharmaceutically acceptable salt of the stereoisomer thereof.

21. A method of treating a cancer in a patient, comprising administering a therapeutically effective amount of the compound according to claim 2, to a patient, thereby treating the cancer.

22. The method of claim 21, in which the compound is selected from the group consisting of:
(S)-4-(2-carbamoyl-5-chloro-3-(2-(phenoxymethyl)morpholinosulfonyl)-1H-indol-7-ylamino)-4-oxobutanoic acid;
(S)-5-(2-carbamoyl-5-chloro-3-(2-(phenoxymethyl)morpholinosulfonyl)-1H-indol-7-ylamino)-3,3-dimethyl-5-oxopentanoic acid;
(S)-4-(2-carbamoyl-5-chloro-3-(2-(phenoxymethyl)morpholinosulfonyl)-1H-indol-7-ylamino)-2,2-dimethyl-4-oxobutanoic acid;
(S)-5-(2-carbamoyl-5-chloro-3-(2-(phenoxymethyl)morpholinosulfonyl)-1H-indol-7-ylamino)-5-oxopentanoic acid;
2-(2-carbamoyl-5-chloro-3-((S)-2-(phenoxymethyl)morpholinosulfonyl)-1H-indol-7-ylcarbamoyl)cyclopropanecarboxylic acid;
(S)-5-chloro-7-(5-morpholino-5-oxopentanamido)-3-(2-(phenoxymethyl)morpholinosulfonyl)-1H-indole-2-carboxamide;
(S)-5-chloro-7-(2-cyanoacetamido)-3-(2-(phenoxymethyl)morpholinosulfonyl)-1H-indole-2-carboxamide;
(S)-ethyl 5-(2-carbamoyl-5-chloro-3-(2-(phenoxymethyl)morpholinosulfonyl)-1H-indol-7-ylamino)-5-oxopentanoate;
(S)-3-(2-carbamoyl-5-chloro-3-(2-(phenoxymethyl)morpholinosulfonyl)-1H-indol-7-ylamino)propanoic acid;
(S)-7-(3-amino-3-oxopropylamino)-5-chloro-3-(2-(phenoxymethyl)morpholinosulfonyl)-1H-indole-2-carboxamide;
(S)-ethyl 4-(2-carbamoyl-5-chloro-3-(2-(phenoxymethyl)morpholinosulfonyl)-1H-indol-7-ylamino)butanoate;
(S)-5-chloro-7-(2-cyanoethylamino)-3-(2-(phenoxymethyl)morpholinosulfonyl)-1H-indole-2-carboxamide;
(S)-5-chloro-3-(2-(phenoxymethyl)morpholinosulfonyl)-7-(tetrahydro-2H-pyran-4-ylamino)-1H-indole-2-carboxamide;
(S)-5-chloro-7-(cyclohexylamino)-3-(2-(phenoxymethyl)morpholinosulfonyl)-1H-indole-2-carboxamide;
(S)-5-chloro-7-(cyclohexylmethylamino)-3-(2-(phenoxymethyl)morpholinosulfonyl)-1H-indole-2-carboxamide;
(S)-methyl 4-((2-carbamoyl-5-chloro-3-(2-(phenoxymethyl)morpholinosulfonyl)-1H-indol-7-ylamino)methyl)benzoate;
(S)-5-chloro-7-(cyclopentylamino)-3-(2-(phenoxymethyl)morpholinosulfonyl)-1H-indole-2-carboxamide;
(S)-7-((1-aminocyclopentyl)methylamino)-5-chloro-3-(2-(phenoxymethyl)morpholinosulfonyl)-1H-indole-2-carboxamide;
(S)-4-((2-carbamoyl-5-chloro-3-(2-(phenoxymethyl)morpholinosulfonyl)-1H-indol-7-ylamino)methyl)benzoic acid;
(S)-7-(1-(tert-butylcarbamoyl)piperidin-4-ylamino)-5-chloro-3-(2-(phenoxymethyl)morpholinosulfonyl)-1H-indole-2-carboxamide;
(S)-5-chloro-7-(1-(cyclohexylcarbamoyl)piperidin-4-ylamino)-3-(2-(phenoxymethyl)morpholinosulfonyl)-1H-indole-2-carboxamide;
(S)-5-chloro-7-(1-(cyclohexylmethylcarbamoyl)piperidin-4-ylamino)-3-(2-(phenoxymethyl)morpholinosulfonyl)-1H-indole-2-carboxamide;
(S)-5-chloro-7-(4-fluorobenzylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-chloro-3-((S)-2-(phenoxymethyl)morpholinosulfonyl)-7-(pyrrolidin-3-ylamino)-1H-indole-2-carboxamide;
(S)-ethyl 4-(2-carbamoyl-5-fluoro-3-(2-(phenoxymethyl)morpholinosulfonyl)-1H-indol-7-ylamino)piperidine-1-carboxylate;
(S)-ethyl 4-(2-carbamoyl-5-chloro-3-(2-(phenoxymethyl)morpholinosulfonyl)-1H-indol-7-ylamino)piperidine-1-carboxylate;
(S)-5-chloro-3-(2-(phenoxymethyl)morpholinosulfonyl)-7-(3-phenylthioureido)-1H-indole-2-carboxamide; and
(S)-5-chloro-3-(2-(phenoxymethyl)morpholinosulfonyl)-7-(piperidin-4-ylamino)-1H-indole-2-carboxamide;
or a stereoisomer thereof;
or a pharmaceutically acceptable salt thereof;
or a pharmaceutically acceptable salt of the stereoisomer thereof.
